(19) United States
(12) Patent Application Publication
(10) Pub. No.: US 2022/0330882 A1
(43) Pub. Date: Oct. 20, 2022

(54) INDIVIDUALIZED SWEAT RATE AND HYDRATION ALERT SYSTEM

(71) Applicant: Kenzen, Inc., Fremont, CA (US)

(72) Inventors: Skip Thomas Orvis, Columbia Falls, MT (US); Claude Vincent Robotham, Tallahassee, FL (US); Nora Elam Levinson, New York, NY (US); Nicole Elizabeth Moyen, San Jose, CA (US); Priyanka Anand Joshi, Dallas, TX (US); Rohit Chandrashekhar Bapat, Raleigh, NC (US)

(21) Appl. No.: 17/233,308

(22) Filed: Apr. 16, 2021

(51) Int. Cl. A61B 5/00; G16H 10/60; G16H 20/30; A61B 5/0205; A61B 5/11

(52) U.S. Cl. CPC A61B 5/4266; G16H 10/60; G16H 20/30; A61B 5/742; A61B 5/02055; A61B 5/6801; A61B 5/1118; A61B 2560/0252; A61B 2562/029

(57) ABSTRACT

A method according to one embodiment includes receiving baseline health data of a user, receiving activity-based health data of a user collected by a sensor device worn by the user while participating in physical activity, and receiving environmental-based data of the user collected by the sensor device worn by the user and/or from an external application programming interface (API). A personalized schedule is generated for the user to follow while participating in the physical activity based on the baseline health data of the user, the activity-based health data of the user and the environmental-based data of the user. The personalized schedule includes at least one instruction of an amount of fluids for the user to consume while participating in the physical activity to maintain hydration of the user and prevent dehydration of the user. The method further includes outputting the personalized schedule for display on the user device.

### FIELD OF THE INVENTION

The present invention relates to environmental conditions, and more particularly, this invention relates to mitigation of productivity losses and injuries experienced as a result of dehydration.

### BACKGROUND

Heat-related injuries and illnesses cost billions of dollars around the world each year in medical care and productivity losses. For example, 30% of individuals who work in relatively elevated temperature environments report productivity losses. A majority of heat-related injuries are mitigated with proper rest and recovery, however, exertional heat stroke and death can occur in some instances as a result of a worker's core body temperature reaching certain elevated levels and/or as a result of dehydration of a worker.

### SUMMARY

A method according to one embodiment includes receiving baseline health data of a user, receiving activity-based health data of a user collected by a sensor device worn by the user while participating in physical activity, and receiving environmental-based data of the user collected by the sensor device worn by the user and/or from an external application programming interface (API). A personalized schedule is generated for the user to follow while participating in the physical activity based on the baseline health data of the user, the activity-based health data of the user and the environmental-based data of the user. The personalized schedule includes at least one instruction of an amount of fluids for the user to consume while participating in the physical activity to maintain hydration of the user and prevent dehydration of the user. The method further includes outputting the personalized schedule for display on the user device.

A computer program product according to another embodiment includes a computer readable storage medium having stored thereon computer readable program instructions configured to cause a processor of a computer system to perform the foregoing method.

A system according to another embodiment includes a processor, and logic integrated with and/or executable by the processor. The logic is configured to perform the foregoing method.

Other aspects and advantages of the present invention will become apparent from the following detailed description, which, when taken in conjunction with the drawings, illustrate by way of example the principles of the invention.

### BRIEF DESCRIPTION OF THE DRAWINGS

FIG. 1 is an architecture, in accordance with one embodiment.

FIG. 2 is a representative hardware environment, in accordance with one embodiment.

FIG. 3 is a flowchart of a method, in accordance with one embodiment.

FIG. 4A is a first state of a touch-sensitive input area of a display of a user device, in accordance with one embodiment.

FIG. 4B is a second state of the touch-sensitive input area of the display of the user device of FIG. 4A.

FIG. 4C is a third state of the touch-sensitive input area of the display of the user device of FIGS. 4A-4B.

FIG. 5 is an architecture, in accordance with one embodiment.

FIG. 6 is an illustration of a user wearing various forms of personal protective equipment (PPE), in accordance with one embodiment.

FIG. 7A is a flowchart of a method, in accordance with one embodiment.

FIG. 7B is a flowchart of a plurality of sub-operations of an operation of the method of FIG. 7A.

FIG. 8 is a flowchart of a method, in accordance with one embodiment.

FIG. 9 is a flowchart of a method, in accordance with one embodiment.

FIG. 10A is a plot, in accordance with one embodiment.

FIG. 10B is a plot, in accordance with one embodiment.

FIG. 11A is a flowchart of a method, in accordance with one embodiment.

FIG. 11B is a flowchart of a plurality of sub-operations of an operation of the method of FIG. 11A.

FIG. 12 is a system, in accordance with one embodiment.

FIG. 13 is a system, in accordance with one embodiment.

FIG. 14 is a flowchart of a method, in accordance with one embodiment.

FIG. 15 is a flowchart of a method, in accordance with one embodiment.

FIG. 16 is a flowchart of a method, in accordance with one embodiment.

### DETAILED DESCRIPTION

The following description is made for the purpose of illustrating the general principles of the present invention and is not meant to limit the inventive concepts claimed herein. Further, particular features described herein can be used in combination with other described features in each of the various possible combinations and permutations.

Unless otherwise specifically defined herein, all terms are to be given their broadest possible interpretation including meanings implied from the specification as well as meanings understood by those skilled in the art and/or as defined in dictionaries, treatises, etc.

It must also be noted that, as used in the specification and the appended claims, the singular forms “a,” “an” and “the” include plural referents unless otherwise specified.

The following description discloses several preferred embodiments of mitigating productivity losses and injuries experienced as a result of environmental conditions and/or related systems and methods.

In one general embodiment, a method includes receiving baseline health data of a user, receiving activity-based health data of a user collected by a sensor device worn by the user while participating in physical activity, and receiving environmental-based data of the user collected by the sensor device worn by the user and/or from an external application programming interface (API). A personalized schedule is generated for the user to follow while participating in the physical activity based on the baseline health data of the user, the activity-based health data of the user and the environmental-based data of the user. The personalized schedule includes at least one instruction of an amount of fluids for the user to consume while participating in the physical activity to maintain hydration of the user and prevent dehydration of the user. The method further includes outputting the personalized schedule for display on the user device.

In another general embodiment, a computer program product includes a computer readable storage medium having stored thereon computer readable program instructions configured to cause a processor of a computer system to perform the foregoing method.

In yet another general embodiment, a system includes a processor, and logic integrated with and/or executable by the processor. The logic is configured to perform the foregoing method.

The description herein is presented to enable any person skilled in the art to make and use the invention and is provided in the context of particular applications of the invention and their requirements. Various modifications to the disclosed embodiments will be readily apparent to those skilled in the art and the general principles defined herein may be applied to other embodiments and applications without departing from the spirit and scope of the present invention. Thus, the present invention is not intended to be limited to the embodiments shown, but is to be accorded the widest scope consistent with the principles and features disclosed herein.

In particular, various embodiments of the invention discussed herein are implemented using the Internet as a means of communicating among a plurality of computer systems. One skilled in the art will recognize that the present invention is not limited to the use of the Internet as a communication medium and that alternative methods of the invention may accommodate the use of a private intranet, a Local Area Network (LAN), a Wide Area Network (WAN) or other means of communication. In addition, various combinations of wired, wireless (e.g., radio frequency), audio modulation and/or optical communication links may be utilized.

The program environment in which one embodiment of the invention may be executed illustratively incorporates one or more general-purpose computers or special-purpose devices such hand-held or body worn computers. Details of such devices (e.g., processor, memory, data storage, input and output devices) are well known and are omitted for the sake of clarity.

It should also be understood that the techniques of the present invention might be implemented using a variety of technologies. For example, the methods described herein may be implemented in software running on a computer system, or implemented in hardware utilizing one or more processors and logic (hardware and/or software) for performing operations of the method, application specific integrated circuits, programmable logic devices such as Field Programmable Gate Arrays (FPGAs), and/or various combinations thereof. In one illustrative approach, methods described herein may be implemented by a series of computer-executable instructions residing on a storage medium such as a physical (e.g., non-transitory) computer-readable medium. In addition, although specific embodiments of the invention may employ object-oriented software programming concepts, the invention is not so limited and is easily adapted to employ other forms of directing the operation of a computer.

The invention can also be provided in the form of a computer program product comprising a computer readable storage or signal medium having computer code thereon, which may be executed by a computing device (e.g., a processor) and/or system. A computer readable storage medium can include any medium capable of storing computer code thereon for use by a computing device or system, including optical media such as read only and writeable CD and DVD, magnetic memory or medium (e.g., hard disk drive, tape), semiconductor memory (e.g., FLASH memory and other portable memory cards, etc.), firmware encoded in a chip, etc.

A computer readable signal medium is one that does not fit within the aforementioned storage medium class. For example, illustrative computer readable signal media communicate or otherwise transfer transitory signals within a system, between systems e.g., via a physical or virtual network, etc.

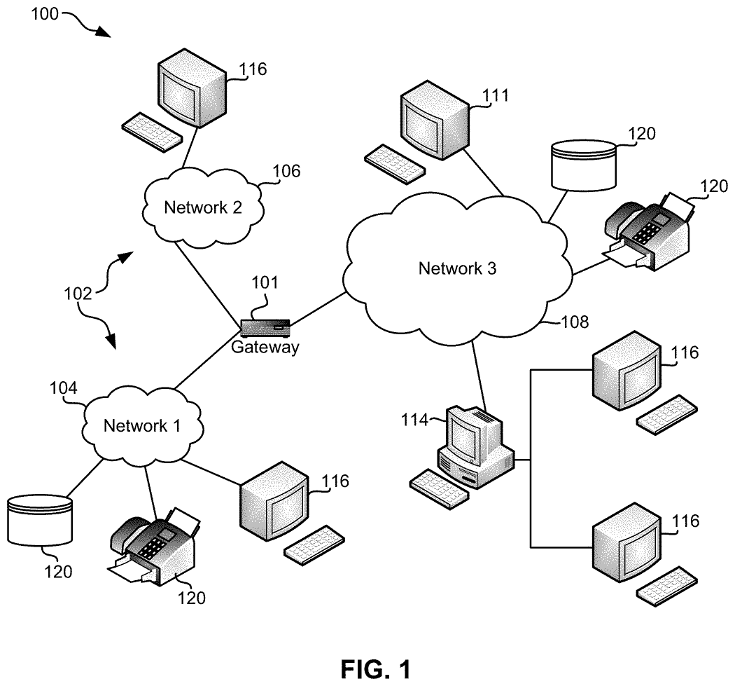

FIG. 1 illustrates an architecture 100, in accordance with one embodiment. As an option, the present architecture 100 may be implemented in conjunction with features from any other embodiment listed herein, such as those described with reference to the other FIGS. Of course, however, such architecture 100 and others presented herein may be used in various applications and/or in permutations which may or may not be specifically described in the illustrative embodiments listed herein. Further, the architecture 100 presented herein may be used in any desired environment.

As shown in FIG. 1, a plurality of remote networks 102 are provided including a first remote network 104 and a second remote network 106. A gateway 101 may be coupled between the remote networks 102 and a proximate network 108. In the context of the present network architecture 100, the networks 104, 106 may each take any form including, but not limited to a LAN, a WAN such as the Internet, public switched telephone network (PSTN), internal telephone network, etc.

In use, the gateway 101 serves as an entrance point from the remote networks 102 to the proximate network 108. As such, the gateway 101 may function as a router, which is capable of directing a given packet of data that arrives at the gateway 101, and a switch, which furnishes the actual path in and out of the gateway 101 for a given packet.

Further included is at least one data server 114 coupled to the proximate network 108, and which is accessible from the remote networks 102 via the gateway 101. It should be noted that the data server(s) 114 may include any type of computing device/groupware. Coupled to each data server 114 is a plurality of user devices 116. Such user devices 116 may include a desktop computer, laptop computer, hand-held computer, printer or any other type of logic. It should be noted that a user device 111 may also be directly coupled to any of the networks, in one embodiment.

A peripheral 120 or series of peripherals 120, e.g., facsimile machines, printers, networked storage units, etc., may be coupled to one or more of the networks 104, 106, 108. It should be noted that databases, servers, and/or additional components may be utilized with, or integrated into, any type of network element coupled to the networks 104, 106, 108. In the context of the present description, a network element may refer to any component of a network.

According to some approaches, methods and systems described herein may be implemented with and/or on virtual systems and/or systems which emulate one or more other systems, such as a UNIX system which emulates a MAC OS environment, a UNIX system which virtually hosts a MICROSOFT WINDOWS environment, a MICROSOFT WINDOWS system which emulates a MAC OS environment, etc. This virtualization and/or emulation may be enhanced through the use of VMWARE software, in some embodiments.

In more approaches, one or more networks 104, 106, 108, may represent a cluster of systems commonly referred to as a “cloud.” In cloud computing, shared resources, such as processing power, peripherals, software, data processing and/or storage, servers, etc., are provided to any system in the cloud, preferably in an on-demand relationship, thereby allowing access and distribution of services across many computing systems. Cloud computing typically involves an Internet or other high speed connection (e.g., 4G LTE, fiber optic, etc.) between the systems operating in the cloud, but other techniques of connecting the systems may also be used.

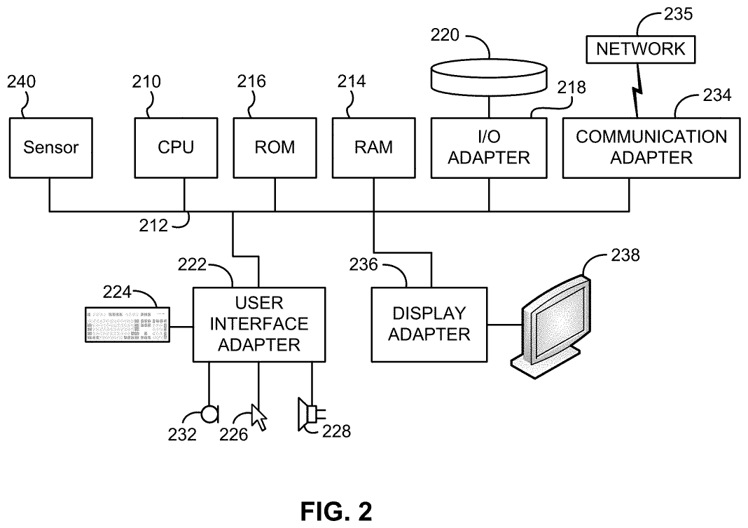

FIG. 2 shows a representative hardware environment associated with a user device 116 and/or server 114 of FIG. 1, in accordance with one embodiment. Such figure illustrates a typical hardware configuration of a workstation having a central processing unit 210, such as a microprocessor, and a number of other units interconnected via a system bus 212.

The workstation shown in FIG. 2 includes a Random Access Memory (RAM) 214, Read Only Memory (ROM) 216, an I/O adapter 218 for connecting peripheral devices such as disk storage units 220 to the bus 212, a user interface adapter 222 for connecting a keyboard 224, a sensing element within a sensor device 240, a mouse 226, a speaker 228, a microphone 232, and/or other user interface devices such as a touch screen and a digital camera (not shown) to the bus 212, communication adapter 234 for connecting the workstation to a communication network 235 (e.g., a data processing network) and a display adapter 236 for connecting the bus 212 to a display device 238.

The workstation may have resident thereon an operating system such as the Microsoft WINDOWS Operating System (OS), a MAC OS, a UNIX OS, etc. It will be appreciated that a preferred embodiment may also be implemented on platforms and operating systems other than those mentioned. A preferred embodiment may be written using JAVA, XML, C, and/or C++ language, or other programming languages, along with an object oriented programming methodology. Object oriented programming (OOP), which has become increasingly used to develop complex applications, may be used. Moreover, a system according to various embodiments may include a processor and logic integrated with and/or executable by the processor, the logic being configured to perform one or more of the process steps recited herein. By integrated with, what is meant is that the processor has logic embedded therewith as hardware logic, such as an application specific integrated circuit (ASIC), a FPGA, etc. By executable by the processor, what is meant is that the logic is hardware logic; software logic such as firmware, part of an operating system, part of an application program; etc., or some combination of hardware and software logic that is accessible by the processor and configured to cause the processor to perform some functionality upon execution by the processor. Software logic may be stored on local and/or remote memory of any memory type, as known in the art. Any processor known in the art may be used, such as a software processor module and/or a hardware processor such as an ASIC, a FPGA, a central processing unit (CPU), an integrated circuit (IC), a graphics processing unit (GPU), etc.

Personalized schedules for displaying to a user participating in activity to thereby mitigate productivity losses and user injuries and/or illnesses

As mentioned elsewhere above, heat-related injuries and illnesses cost billions of dollars around the world each year in medical care and productivity losses. For example, 30% of individuals who work in relatively elevated temperature environments report productivity losses. A majority of heat-related injuries are mitigated with proper rest and recovery, however, exertional heat stroke and death can occur in some instances as a result of a worker's core body temperature reaching certain elevated levels. For context, for each 1 degree Fahrenheit increase in summer temperatures, the likelihood of heat-related deaths increases up to 37%. Many of these heat-related problems result from workers over-exerting themselves because either a) workers themselves do not know when they need to stop working and take a break and/or b) the managers of workers do not know when to instruct workers to stop working and take a break, etc. Most often these heat injuries and illnesses occur while working in a relatively hot work environment and/or wearing heavy protective gear (e.g. PPE) that prohibits heat loss.

Many heat-related injuries and illnesses occur despite managers utilizing work shift schedules. This is because these schedules only consider a limited number of variables among workers, and ultimately do not account for the large physiological variability among individuals, e.g., biological sex, age, fitness level, acclimatization status, whether the worker is taking medication, what type and/or dosage of medication a worker is taking, etc. Moreover, conventional work schedules are often designed from laboratory tests that utilize subject populations predominantly comprised of young, healthy men. Accordingly, typical conventional schedules are not accurate for women and older individuals, e.g., such as women more than thirty-five years old.

Various embodiments and approaches described herein mitigate the heat-related injuries and illnesses described above and furthermore enable productivity of one or more users by generating a personalized schedule for a user to follow while participating in physical activity. The personalized schedule is based on baseline health data, activity-based health data of the user and user feedback.

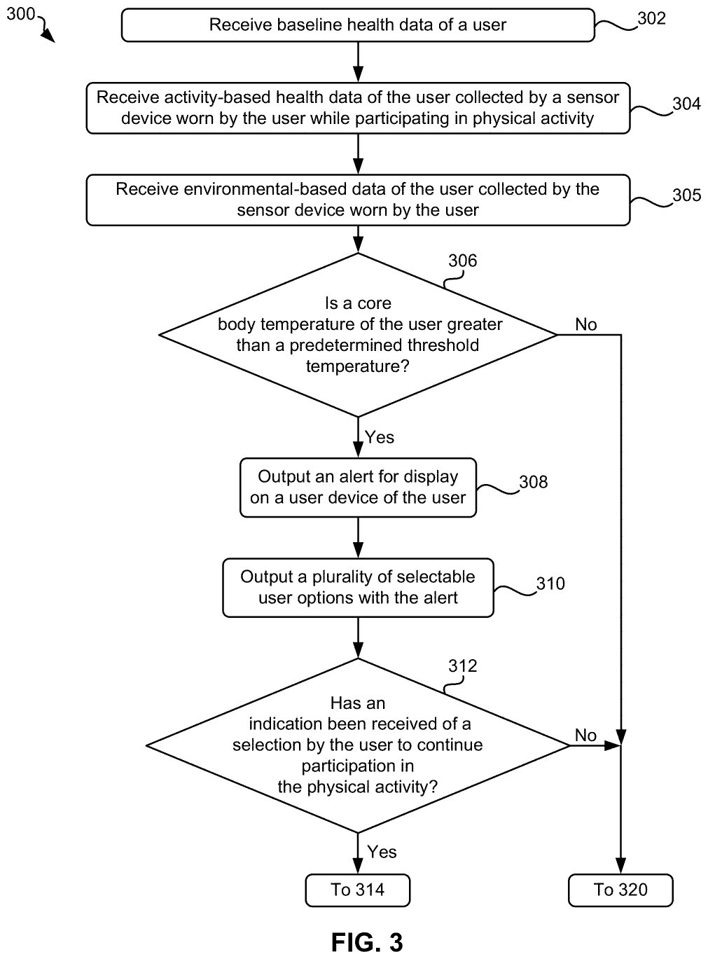

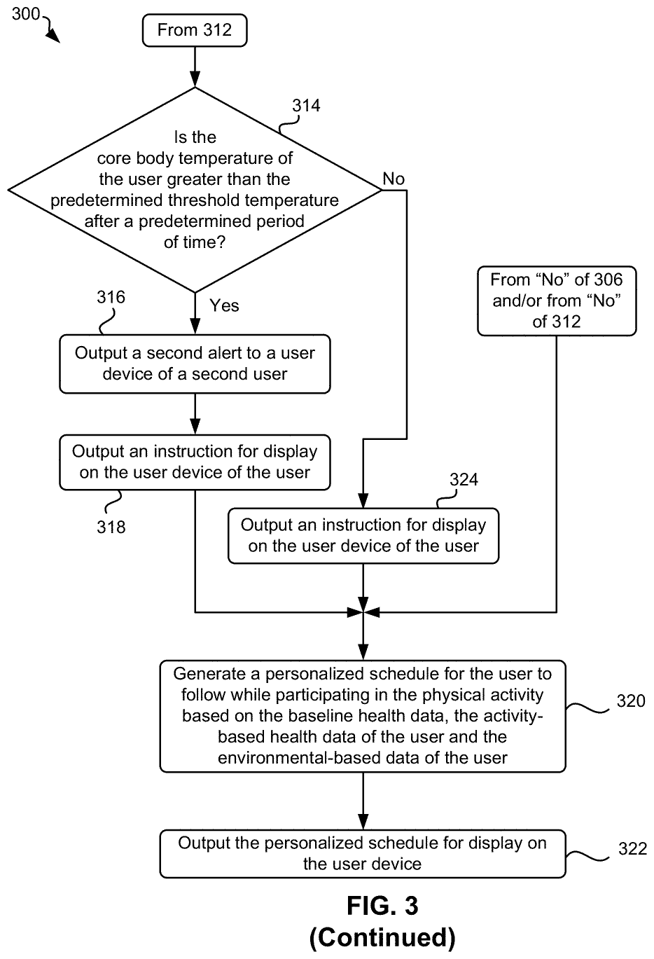

FIG. 3 shows a method 300, in accordance with one embodiment. As an option, the present method 300 may be implemented in devices such as those shown in the other FIGS. described herein. Of course, however, this method 300 and others presented herein may be used to provide applications which may or may not be related to the illustrative embodiments listed herein. Further, the methods presented herein may be carried out in any desired environment. Moreover, more or less operations than those shown in FIG. 3 may be included in method 300, according to various embodiments. It should also be noted that any of the aforementioned features may be used in any of the embodiments described in accordance with the various methods.

For context, it may be prefaced that method 300 includes techniques for generating a personalized schedule for a user to follow while participating in physical activity. As a result of generating and displaying the personalized schedule to the user, the user is instructed when to start participating in the physical activity and when to stop participating in the physical activity, where the instructions prevent the user from being injured or becoming ill as a result of the environmental conditions experienced by the user while participating in the physical activity.

Operation 302 of method 300 includes receiving baseline health data of a user. Note that the received baseline health data may be pre-acquired, e.g., using a known type of health data input module, using a known technique for gathering health data from one or more sources, based on one or more questionnaires, processing images of a user using one or more known types of recognition techniques, etc. A non-limiting list of the baseline health data of the user may include, e.g., medical history data, anthropometric data such as weight and/or height, age, biological sex, a determined body mass index, etc.

Activity-based health data of the user, e.g., biometric data, collected by a sensor device worn by the user while participating in physical activity is received, e.g., see operation 304 of method 300. Accordingly, in some approaches, the sensor device may be configured to continually, e.g., according to a predetermined interval, and non-invasively, collect predetermined metrics of the user using one or more known types of sensors, e.g., a camera, a heartbeat sensor, a temperature sensor for determining a temperature of the user, a humidity sensor, a motion sensor, a global positioning system, a proximity sensor, a gyroscope, an accelerometer, a microphone, etc. The activity-based health data may depend on the approach. For example, in some approaches, the activity-based health data may include physiological data of the user, e.g., ambient temperature, ambient humidity, skin temperature of the user, skin humidity of the user measured by the sensor device, near skin humidity of the sensor, a perspiration rate of the user, a heart rate of the user such as measured via photoplethysmography or via one or more known techniques, an activity type that the user is determined to be engaged in and data associated therewith, a rate of movement of the user, etc.

Environmental-based data of the user collected by the sensor device worn by the user is additionally and/or alternatively received, e.g., see operation 305. The environmental-based data may be based on an environment that the user is participating in physical activity in and/or an environment that the user is participating in stationary activity in, e.g., workers who are sedentary but are working in hot environments such as a worker driving a tractor or operating machinery, relatively older adults who may be experiencing heat stress such as due to a heat wave and could be alerted about their core temp to avoid heat stroke, etc. For example, a non-limiting list of environmental-based data that may be received includes a geographical location of the user, a humidity percentage, a microclimate temperature and relative humidity, radiative heat experienced by the user, weather data of a geographical location of the user such as based on a weather forecast and/or an Application Programming Interface (API) call to a weather source, etc. Depending on the approach, the environmental-based data of the user may be collected by the sensor device using one or more known types of components configured to collect environmental-based data, e.g., an ambient light sensor, a barometer, a compass, an antenna, a camera, a humidity sensor, an optical light detector, a microphone, etc.

Data based on clothing worn by the user and/or clothing expected to be worn by the user may additionally and/or alternatively be received in an optional operation of method 300. For example, in one approach, method 300 may include outputting a questionnaire for the user to input one or more clothing layers, e.g., one layer of clothing, two layers of clothing, three layers of clothing, etc., that user is wearing and/or plans to wear within a predetermined period of time, e.g., during a scheduled work shift of the user, during a particular day of the week, in one or more anticipated weather patterns as specified in the questionnaire, etc. Note that the data based on clothing of the user may additionally and/or alternatively be pre-acquired, e.g., using a known type of data input module, using a known technique for gathering clothing data from one or more sources, processing images of a user using one or more known types of recognition techniques, etc.

The sensor device worn by the user while participating in physical activity may include one or more components, depending on the approach. In one preferred approach, the sensor may be worn on an upper arm region, e.g., between the user's elbow and shoulder socket, between the user's bicep and shoulder socket, on about a center of the user's triceps muscle, at about a base of the user's deltoid muscle, etc. In another approach, the sensor device may additionally and/or alternatively include a component that is configured to be worn on a lower arm region of the user, e.g., between the user's elbow and wrist, on about a user's wrist, on a user's elbow, etc. In yet another approach, the sensor device may additionally and/or alternatively include a component that is configured to be worn on another body part of the user, on the user's chest, on the user's leg(s), on the user's feet, on the user's face, etc. The sensor may be worn by the user using one or more known types of attachment components, e.g., hook and loop straps, adhesives, magnets, hook-and-loop fastener, tape, clips, elastic bands, etc.

It should be noted that the type of physical activity may depend on the approach. For example, in one preferred approach, the physical activity may be work-related physical activity. In another approach, the physical activity may be exercise-based physical activity. In yet another approach, the physical activity may be physical therapy-based physical activity, which may include use of a sauna. In yet another approach, the physical activity may be military-based physical activity, e.g., such as performing a mission, training in a military camp, dealing with traumatic events subsequent to experiencing such evens, etc. In another approach, the physical activity may be education physical activity such as physical activity that occurs while a student is attending school. In some approaches, participation in the physical activity may be detected based on one or more factors, e.g., in response to a determination that the user is physically active at a predetermined geographical location, in response to a determination that the user is physically active within predefined work hours, in response to a determination that the user is physically active during a predetermined period of a day, in response to a determination that the user is physically active subsequent to the user clocking-in to work and immediately prior to the user clocking-out of work, etc.

Although various approaches described elsewhere above include the activity-based health data being collected by the sensor device worn by the user while participating in physical activity, e.g., such as work-related physical activity, in contrast, in some approaches, the activity-based health data may be collected by the sensor device worn by the user while participating in stationary activity, e.g., sitting at a desk, sitting on a chair at a recovery center, stationary behavior in a retirement center, etc. The user's activity-based health data may in some approaches be collected by the sensor device worn by the user while participating in stationary activity because even stationary activity can lead to the user's core body temperature being greater than a predetermined threshold. For example, in some cases, elderly people, who are sedentary but experiencing a heat wave, may experience a core body temperature that is greater than a predetermined threshold. Accordingly, in some approaches the activity-based health data may incorporate even stationary activity, e.g., to be monitored for and prevent heat illness/heat stroke.

As a result of the user participating in activity, e.g., physical activity, stationary activity, etc., a core body temperature of the user may increase. In order to prevent the user from continuing to participate in the physical activity to an extent that would otherwise result in bodily harm to the user, it may be determined whether the core body temperature of the user greater than a predetermined threshold temperature and/or a rate of change in core body temperature of the user exceeds a predetermined “safe” rate, e.g., see decision 306 of method 300. Note that the predetermined threshold temperature may be any temperature, and may be set and/or adjusted by one or more input sources, e.g., a manager of the user, an employer of the user, a doctor of the user, a global and/or national health committee, the user, etc. In some preferred approaches, the predetermined threshold temperature is in a range of about 37.8 degrees Celsius to about 38.6 degrees Celsius. However, the predetermined threshold temperature may be set and/or adjusted outside of this range.

In response to a determination that the core body temperature of the user is not greater than the predetermined threshold temperature (e.g., as illustrated by the “No” logical path of decision 306) and/or in response to a determination that the rate of change in core body temperature of the user does not exceed a predetermined safe rate, method 300 optionally continues to operation 320. In some approaches, in response to the determination that the core body temperature of the user is not greater than the predetermined threshold temperature, it may be determined that the user continuing to participate in the physical activity is unlikely to result in an environmental condition-based illness and/or injury. Accordingly, in one or more of such approaches, the user is not notified and/or alerted to the relatively safe user condition. In contrast, in some optional approaches, in response to the determination that the core body temperature of the user is not greater than the predetermined threshold temperature, a status indicator may be output for display and/or audible broadcast on a user device, e.g., such as a computer of the user, a tablet device of the user, a phone of the user, a television of the computer, glasses of the user that are configured to display at least some information to the user, etc. In some approaches, the status indicator may additionally and/or alternatively be output to the sensor device of the user to notify and/or alert the user of the relatively safe user condition. For example, in one or more of such approaches, the sensor device may include a haptic motor or visual indicator that is configured to alert the user of the relatively safe user condition, e.g., by a predetermined vibration or visible pattern that is known and recognizable to the user.

In contrast, in some approaches, in response to a determination that the core body temperature of the user is greater than the predetermined threshold temperature (e.g., as illustrated by the “Yes” logical path of decision 306) and/or in response to a determination that the rate of change in core body temperature of the user exceeds a predetermined safe rate, the user may be notified and/or alerted to the relatively unsafe user condition. For context, the user may be notified and/or alerted to the relatively unsafe user condition in order to enable the user to recover, e.g., lower the core body temperature of the user, before potentially experiencing an injury and/or illness as a result continuing to participate in the physical activity while having a core body temperature of the user is greater than the predetermined threshold temperature. Such an alert may assist the user in avoiding injury and/or illness because many users that are injured and/or become ill as a result of participating in a physical activity do not realize that they should stop to lower their core body temperature until it is too late to do so, e.g., until injury and/or illness is unavoidable. An alert is in some approaches output for display on the user device in response to a determination that a core body temperature of the user is greater than a predetermined threshold temperature, e.g., see operation 308 of method 300. The alert may instruct the user to stop participating in the physical activity. In some approaches, information may be included in the alert. The information may assist the user in lowering their core body temperature. For example, the information of the alert may include, e.g., an instruction to “Stop Work,” suggestions of techniques to potentially lower the core body temperature of the user, location information of a nearest hydration source and/or cooling station, etc. In some approaches, an estimated recovery time, which may be calculated using one or more known techniques for determining a recovery time, may be included in the alert. The information of the alert may additionally and/or alternatively include an estimation of a type of physical activity that resulted in the core body temperature of the user being greater than a predetermined threshold temperature. In some approaches, such an estimation may be based on determined relative strenuous scores of activities that the user has performed during a predetermined period of participation in the physical activity. Such determinations may be based on the received activity-based health data of the user. For example, a physical activity that includes walking on a level surface within the predetermined period of participation in the physical activity may be determined to have and assigned a lower relative strenuous score than climbing a flight of stairs. In such an example, climbing the flight of stairs may be the estimated type of physical activity that resulted in the core body temperature of the user being greater than the predetermined threshold temperature based on climbing the flight of stairs having a greater relative strenuous score than the relative strenuous score of walking on a level surface. One or more predetermined suggestions to change clothing layers, if it is deemed (based on the user's physiological sensor data) that their clothing is contributing to core temperatures exceeding the predetermined threshold may be output to the user device, to a user device of a second user that is a supervisor of the user, etc.

The alert may additionally and/or alternatively be output to the sensor device in some approaches. In one or more of such approaches, the sensor device may include a motor that is configured to alert the user to stop participating in the physical activity, e.g., by a predetermined vibration pattern that is known and recognizable to the user to be associated with stopping participating in the physical activity.

Various approaches above describe the alert to be output for display on the user device. In one or more of such approaches, the alert may be output to a touch-sensitive input area of the display of the user device to enable selection of one or more selectable user options output with the alert. Operation 310 includes outputting a plurality of selectable user options with the alert. The outputting of the selectable user options is in some approaches optional, and may enable the user to provide insight as to how the user plans to respond to the alert. For example, in some approaches a first of the selectable user options may correspond to an option to continue participating in the physical activity for a predetermined period of time despite the alert, e.g., continue for one minute, continue for ten minutes, continue for one hour, etc. According to a more specific example, the first of the selectable user options may include the text “I feel OK to work.” In contrast, in some approaches a second of the selectable user options may correspond to an option to, at least temporarily, stop participating in the physical activity. For example, the second of the selectable user options may include the text “I will rest.”

It is determined whether an indication has been received of a selection by the user to continue participation in the physical activity, e.g., see decision 312.

In response to a determination that an indication has not been received of a selection by the user to continue participation in the physical activity (e.g., as illustrated by the “No” logical path of decision 312), method 300 optionally continues to operation 320.

In response to receiving, from the user device, an indication (e.g., as illustrated by the “Yes” logical path of decision 312) of a selection by the user of the first of the selectable user options, e.g., corresponding to the option to continue participating in the physical activity for a predetermined period of time despite the alert, it may be determined, after the predetermined period of time, whether the core body temperature of the user is greater than the predetermined threshold temperature, e.g., see decision 314. In response to a determination that the core body temperature of the user is greater than or equal to the predetermined threshold temperature after the predetermined period of time (e.g., as illustrated by the “Yes” logical path of decision 314) one or more predetermined operations may be performed, e.g., in an effort to protect the wellbeing of the user. It should be prefaced that a determination that the core body temperature of the user is greater than or equal to the predetermined threshold temperature after the predetermined period of time is preferably a matter of concern because it is likely that the user, in electing to continue participating in the activity despite the first alert, misjudged their need to stop participation in the activity to allow their body to lower. Accordingly, depending on the approach, the one or more predetermined operations performed in an effort to protect the wellbeing of the user may constitute a safety escalation path that is performed until it is determined that the core body temperature of the user is not greater than the predetermined threshold temperature. For example, in one approach, a second alert may be output to a user device of a second user in response to the determination that the core body temperature of the user is greater than or equal to the predetermined threshold temperature after the predetermined period of time, e.g., see operation 316. The second alert may alert the second user that the core body temperature of the user is greater than the predetermined threshold temperature and/or that the first user continues to participate in the physical activity while having a core body temperature that is greater than the predetermined threshold temperature, e.g., see operation 316. The second user may in some approaches be an authoritative figure and/or someone with the ability to assist in lowering the core body temperature of the user, e.g., a supervisor of the user at a work site, a doctor of the user, a family member of the user, someone that the user has designated as an emergency contact on medical forms, a co-worker that has experience with a line of work that the user performs such that the second user can cover for the user while the user at least temporarily suspends participation in the physical activity, etc. Note that in such an approach, the second alert may be output to the user device of the second user, because the user may be unable to recognize the injury and/or illness that their continuing to participate in the activity may result in. An instruction may additionally and/or alternatively be output for display on the user device of the user in response to the determination that the core body temperature of the user is greater than or equal to the predetermined threshold temperature after the predetermined period of time, e.g., see operation 318. The instruction preferably instructs the user to stop participating in the physical activity in some approaches. Moreover, the instruction may not include selectable options to continue participating in the physical activity, because doing so may create a false impression to the user that it may be safe for the user to continue such participation. In some approaches, information may be included in the instruction. The information may assist the user in lowering their core body temperature. For example, the information of the instruction may include, e.g., an instruction to “Stop Work,” suggestions of techniques to potentially lower the core body temperature of the user, location information of a nearest hydration source and/or cooling station, etc. In some approaches, an estimated recovery time, which may be based on the received data, may be calculated using one or more predetermined algorithm(s) or known techniques for determining a recovery time, and be included in the instruction. The information of the instruction may additionally and/or alternatively include an estimation of a type of physical activity that resulted in the core body temperature of the user being greater than a predetermined threshold temperature.

It should be noted that the determination of whether the core body temperature of the user is greater than or equal to the predetermined threshold temperature may additionally and/or alternatively be based on the received data. In other words, the determination is preferably not simply based on a timer expiring, but in some approaches rather additionally and/or alternatively a re-evaluation of the user's core body temperature and physical condition that is based on the received data.

In contrast, in response to a determination that the core body temperature of the user is not greater than or equal to the predetermined threshold temperature after the predetermined period of time (e.g., as illustrated by the “No” logical path of decision 314) in some approaches, the user may optionally be prompted to resume participation in the physical activity. For example, in some approaches, in response to a determination that the core body temperature of the user is not greater than the predetermined threshold temperature after the predetermined period of time, an instruction may be output for display on the user device, e.g., see operation 324. The second instruction may instruct the user to optionally resume participation in the physical activity. In some approaches, the user may be only allowed to partially resume participation in the physical activity, so as to prevent the user from being quickly overexerted and thereafter again have a core body temperature that is greater than or equal to the predetermined threshold temperature. In one or more of such approaches, method 300 optionally includes outputting a limited list of predetermined types of physical activity that the user may participate in, e.g., types of physical activity that are predetermined using the baseline health data of the user and/or using the activity-based health data of the user to not be relatively strenuous for the user. In some other approaches, in response to a determination that the core body temperature of the user is not greater than or equal to the predetermined threshold temperature after the predetermined period of time method 300 may optionally additionally and/or alternatively include outputting an indication to the sensor device to alert the user that the user may resume participation in the physical activity. For example, in one or more of such approaches, the indication may be configured to instruct a motor of the sensor device to play a predetermined vibration pattern or a visible alert system of notification blinks that is known and recognizable to the user to be associated with being allowed to resume participation in the physical activity and/or that is known and recognizable to the user to be associated with having a safe core body temperature.

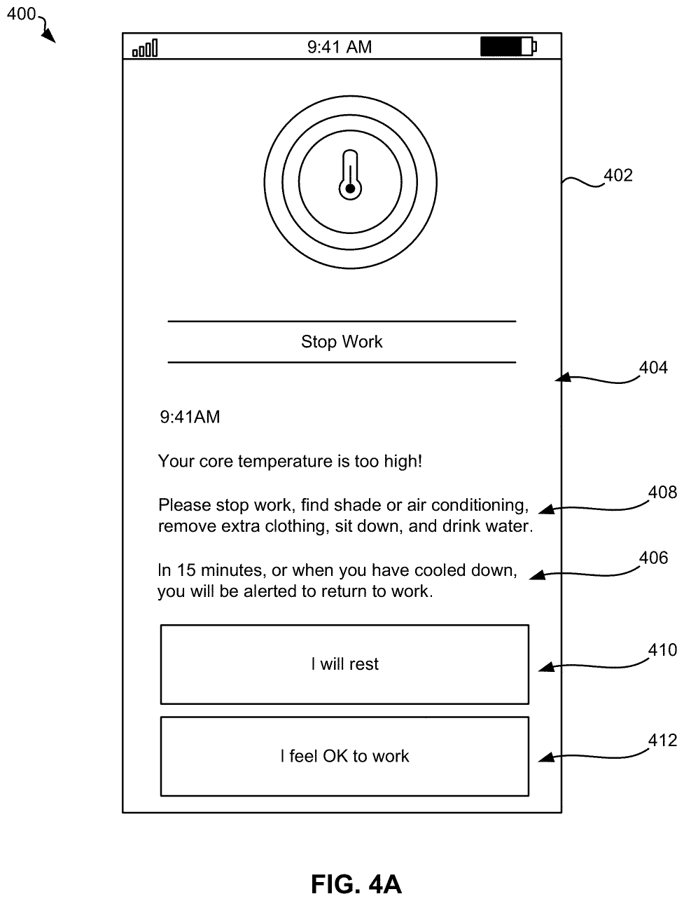

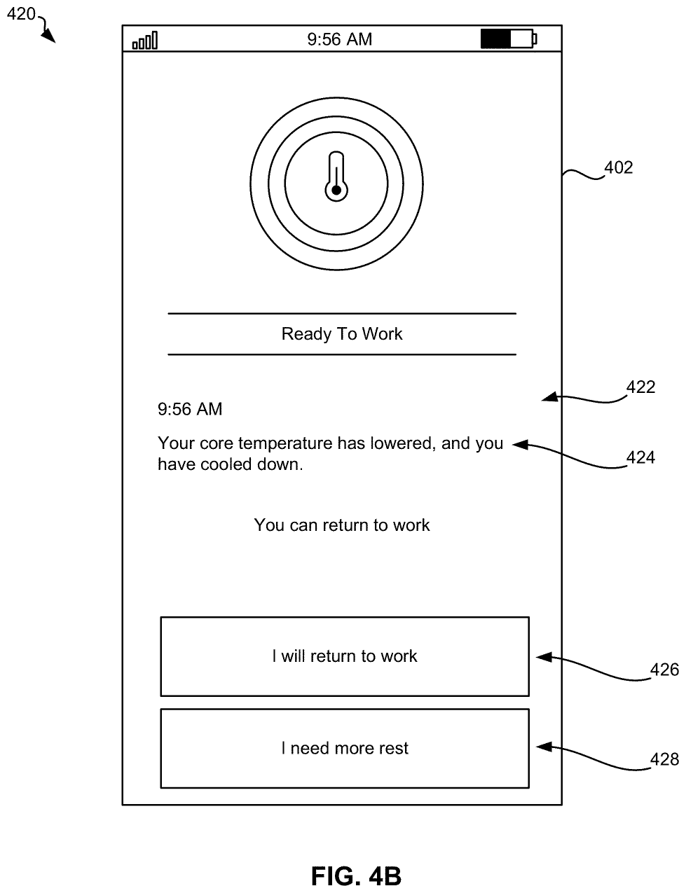

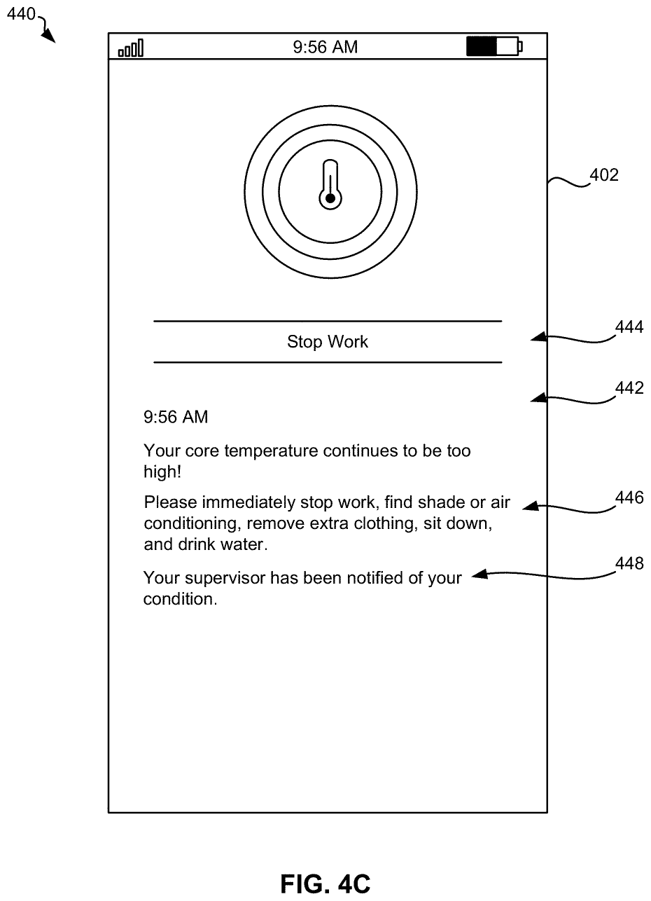

It should be noted that one or more of the alerts described in various approaches above may, in some approaches, be additionally and/or alternatively output to the sensor device, e.g., to notify the user that their core body temperature is greater than the predetermined threshold temperature. Illustritive alerts that may be output in response to a determination that a core body temperature of the user is greater than a predetermined threshold temperature will be described in further detail elsewhere herein, e.g., see FIG. 4A.

Operation 320 of method 300 includes generating a personalized schedule for the user to follow while participating in the physical activity. The personalized schedule may include information that is determined to be likely to prevent the user from experiencing environmental condition-based injury and/or illness when the personalized schedule is followed by the user when participating in the physical activity. For example, the personalized schedule in some approaches includes at least one instruction of when to start participating in the physical activity, e.g., a “Start Work” order, and at least one instruction of when to stop participating in the physical activity, e.g., a “Stop Work” order. For context, at least one instruction of when to start participating in the physical activity may be scheduled for a time that the user is determined and/or estimated to be physically capable of participating in the physical activity, without the user, as a result of the participation in the physical activity, having a core body temperature that is greater than the predetermined threshold temperature. Moreover, at least one instruction of when to stop participating in the physical activity may be scheduled for a time that the user is determined and/or estimated to be physically uncapable of participating in the physical activity, without the user, as a result of continuing participation in the physical activity, having a core body temperature that is greater than the predetermined threshold temperature. Note that at least one instruction of when to stop participating in the physical activity may be scheduled such that the user is instructed to stop participating in the physical activity at any time, e.g., just prior (such as one degree Celsius or less) to the user's core body temperature exceeding the predetermined threshold temperature, when the user's core body temperature matches the predetermined threshold temperature, when the user's core body temperature slightly exceeds the predetermined threshold temperature, etc.

The personalized schedule may be generated using one or more techniques and using one or more types of data. For example, in some preferred approaches, the personalized schedule may be based on the baseline health data, the activity-based health data of the user, and the environmental-based data of the user, e.g., see operation 320. In another approach, the personalized schedule may additionally and/or alternatively be based on data based on clothing worn by the user and/or clothing expected to be worn by the user.

Input received from user interactions with the alerts may additionally and/or alternatively be incorporated into the personalized schedule, e.g., indications received of selection by a user of at least one of a plurality of selectable user options, a frequency in which a user elects to continue working subsequent to an alert being output to instructing the user to stop participating in the physical activity, whether or not a user follows one or more suggestions of techniques to potentially lower the core body temperature of the user, etc.

The personalized schedule may in some approaches be generated using a table that includes a plurality values of when to begin participating in the physical activity and when to stop participating in the physical activity that are pre-associated to values of the received data. For example, in one approach, at least some start and stop instructions of the personalized schedule may be determined by accessing the table and determining which start and stop instructions correspond to values of the received data, e.g., baseline health data of the user, activity-based on health data of the user, environmental-based data of the user, data based on clothing worn by the user and/or clothing expected to be worn by the user, etc.

The personalized schedule may in some approaches additionally and/or alternatively be generated using data modeling. In one or more of such approaches a database may be generated by the data modeling using ongoingly collected and/or updated values of the received data. Data of the database may be comparatively analyzed with the received data of a user in order to determine a personalized schedule for the user. More specifically, in some approaches, the comparative analysis may include comparing data of the user to data of the database to identify start and stop instructions that was previously incorporated into a personalized schedule of a user having data within a predetermined degree of similarity, e.g., which may be the user or another user, and did not result in the user's core body temperature exceeding the predetermined threshold temperature while following the personalized schedule. In such approaches, the more data that is incorporated into the modeling and database, the more likely a generated personalized schedule is to prevent a user's core body temperature from exceeding the predetermined threshold temperature. In some other approaches, one or more known techniques of data modeling may additionally and/or alternatively be incorporated into the generation of the personalized schedule for the user to follow.

In some approaches, the personalized schedule may additionally and/or alternatively be generated based on measured and/or predicted environmental values, e.g., such as the receives environmental-based data of the user collected by the sensor device worn by the user. One or more of such approaches may utilize API calls to a weather source and/or network connected environmental sensors. In one approach, such data may be incorporated into a data modeling technique described above in order to generate the personalized schedule.

Data collection and research based on trial and analysis of results may additionally and/or alternatively be used for generating the personalized schedule for the user to follow. Note that in one or more of such approaches, the trial process may be performed in a lab or any other controlled setting to ensure that users are not injured as a result of following a personalized schedule that is based on a minimal amount of trial and analysis. The trial and analysis of results may in one approach include increasing a period of time between an instruction to start participating in the physical activity and an instruction to stop participating in the physical activity in response to a determination that the user following the personalized schedule does not have a resulting core body temperature that exceeds the predetermined threshold temperature while participating in the physical activity. In contrast, the trial and analysis of results may additionally and/or alternatively include decreasing a period of time between an instruction to start participating in the physical activity and an instruction to stop participating in the physical activity in response to a determination that the a user following the personalized schedule has a resulting core body temperature that exceeds the predetermined threshold temperature while participating in the physical activity.

Test case research and extrapolation based on results may additionally and/or alternatively be used for generating the personalized schedule for the user to follow. For example, in some approaches, the received data of the user may be applied to one or more known techniques of test case research and extrapolation to generate the personalized schedule for the user to follow. One or more known types of calculations may be additionally and/or alternatively be used for generating the personalized schedule for the user to follow. For example, in some approaches, the received data of the user may be applied to a black box equation having an output that includes a personalized schedule for the user to follow. In another approach, the received data of the user may be applied to a known type of machine learning algorithm to generate and/or ongoingly update the personalized schedule for the user to follow.

With continued reference to method 300, operation 322 includes outputting the personalized schedule for display on the user device.

Although various approaches above describe generating a personalized schedule for the user to follow while participating in the physical activity, in some approaches, method 300 optionally additionally and/or alternatively includes generating a physical activity schedule that incorporates a plurality of users including the user. For example, the physical activity schedule that incorporates a plurality of users including the user may in some approaches include at least one instruction of when to start participating in the physical activity and at least one instruction of when to stop participating in the physical activity for each of the plurality of users. In at least some of such approaches, each of the instructions of when to start participating in the physical activity may be based on data, e.g., the baseline health data of the user, the activity-based health data of the user the environmental-based data of the user, clothing data of the user, etc., of a different one of the plurality of users, and each of the instructions of when to stop participating in the physical activity may be based on data of a different one of the plurality of users.

In some approaches, method 300 includes pairing up to a predetermined number of users each having personalized schedules with at least a predetermined degree of similarity with one another in the generated physical activity schedule. This pairing may be particularly useful in work settings in which the users are employees. This is because a first group of workers paired together in the generated physical activity schedule may be assigned to the work shift, e.g., thereby enabling a shift of workers to be replaced (clocked-out from work) by a second group of workers paired together in the generated physical activity schedule at the same time. This pairing format additionally enables productivity in a work environment because the groups of workers paired together are able to take breaks at the same time, e.g., in accordance with the workers' instructions of when to stop participating in the physical activity. Note that the workers taking breaks at different times would otherwise potentially result in a loss of productivity in the event that a plurality of workers are not simultaneously available to work together on a task that requires the plurality of workers to be accomplished.

In contrast, method 300 may additionally and/or alternatively include pairing up to a predetermined number of users each having personalized schedules with at least a predetermined degree of dissimilarity with one another in the generated physical activity schedule, e.g., such as for a predetermined task and/or work assignment. This pairing may be particularly useful in work settings in which the users are employees. This is because the pairing of workers each having personalized schedules with a predetermined degree of dissimilarity with one another in the generated physical activity schedule prevents a place of work from experiencing a shut down, which might otherwise occur if the workers of a shift were all instructed to stop participation in the work shift at the same time. This productivity benefit is enabled based on the instructions of when to stop participating in the physical activity and the instructions of when to start participating in the physical activity of the workers in the pairing differing from one another.

One or more optional operations of method 300 may enable users having personalized schedules with at least a predetermined degree of similarity to trade time slots in which they are scheduled to participate in physical activity, e.g., such as a work shift. For example, method 300 in one approach includes receiving, from a user device of a first user, a request to trade at least one instruction of when to start participating in the physical activity and at least one instruction of when to stop participating in the physical activity with at least one instruction of when to start participating in the physical activity and at least one instruction of when to stop participating in the physical activity of a second user. It may be determined whether the data of the first user has at least a predetermined degree of similarity with the data of the second user. In response to a determination that the data of the first user has at least a predetermined degree of similarity with the data of the second user, the request to trade may be output to a user device of the second user. A response may be received from the user device of the second user and it may be determined whether the response indicates, e.g., indicates in metadata of the response, that the second user has elected to accept the trade request. In response to a determination that the response indicates that the second user has elected to accept the trade request, a physical activity schedule that includes the instructions of the first user and the instructions of the second user may be updated to reflect the trade. Moreover, the updated physical activity schedule may optionally be output for display on one or more user devices, e.g., the user devices of each of the users associated with the instructions included in the updated physical activity schedule and/or the user devices of each of the users associated with the instructions included in a version of the physical activity schedule that existed prior to the updating.

In contrast, in addition and/or as an alternative to the users initiating changes in the generated physical activity schedule, other users, e.g., supervisors, managers, officers, etc., may initiate requests and/or reassignments to shift allocations within the generated physical activity schedule of the plurality of users. For example, the generated physical activity schedule may be displayed on an application on a user device, at which point the other user may which to initiate requests and/or reassignments to shift allocations within the generated physical activity schedule of the plurality of users. In response to receiving the requests and/or reassignments to shift allocations within the generated physical activity schedule of the plurality of users, the physical activity schedule may optionally be updated.

it is important to note that baseline health data of a user, activity-based health data of the user, environmental-based data of the user, clothing based data of the user and user selections of selectable user options have heretofore not been considered and/or incorporated into generated schedules of a user to follow while participating in a physical activity. As mentioned elsewhere above, this is because conventional generated schedules only consider a limited number of variables among workers, and ultimately do not account for the large physiological variability among individuals, e.g., biological sex, age, fitness level, acclimatization status, whether the worker is taking medication, what type and/or dosage of medication a worker is taking, etc. Moreover, conventional work schedules are often designed from laboratory tests that utilize subject populations predominantly comprised of young, healthy men, and therefore, typical conventional schedules are not accurate for women and older individuals, e.g., such as women more than thirty-five years old. Accordingly, the inventive discoveries disclosed herein with regards to the consideration and/or incorporation of baseline health data of a user, activity-based health data of the user, environmental-based data of the user, clothing based data of the user and user selections of selectable user options into generated personalized schedules proceed contrary to conventional wisdom.

It should further be noted that, as a result of incorporating such data into generated personalized schedules, a significant number of injury events and illness events that would otherwise potentially occur as a result of users using conventional schedules that incorporate a limited sample of data will be mitigated. Note that these injuries and/or illnesses result in additional and resource-intensive processing tasks in computer systems associated with the conventional schedules, as adjustments to avoid such injuries and/or illnesses are ongoingly calculated. In sharp contrast, performance of computer systems are improved as a result of the techniques of various embodiments and approaches described herein considering and/or incorporating baseline health data of a user, activity-based health data of the user, and user selections of selectable user options into generated personalized schedules. Note that this improvement is noticed where the computer is configured as a standalone computer and/or a computer in a network of computer devices.

FIGS. 4A-4C depict states 400, 420, 440 of a touch-sensitive input area of a display of a user device, in accordance with various embodiments. As an option, the present states 400, 420, 440 may be implemented in conjunction with features from any other embodiment listed herein, such as those described with reference to the other FIGS. Of course, however, such states 400, 420, 440 and others presented herein may be used in various applications and/or in permutations which may or may not be specifically described in the illustrative embodiments listed herein. Further, the states 400, 420, 440 presented herein may be used in any desired environment.

Referring first to FIG. 4A, the state 400 of a touch-sensitive input area of a display 402 of a user device includes an alert 404 displayed thereon. In one approach, it may be assumed that the alert 404 displayed on the touch-sensitive input area of the display 402 in output for such display in response to a determination that a core body temperature of a user is greater than a predetermined threshold temperature, e.g., see “Your core temperature is too high!” The alert 404 instructs the user to stop participating in the physical activity, e.g., see “Stop Work.” Information is also included in the alert 404. For example, information 406 informs the user that the user may be alerted to return to work in fifteen minutes and/or once it is determined that the core body temperature of the user has is not greater than the predetermined threshold temperature. Note that one or more operations of method 300 may be utilized to perform such a determination, and moreover, it may be noted that such a determination may be based on an individual's physiological sensor data. Moreover, information 408 includes suggestions that may assist the user in lowering their core body temperature. A plurality of selectable user options 410, 412 are output with the alert 404 for display on the display 402. The first selectable user options 412 corresponds to an option to continue participating in the physical activity for a predetermined period of time despite the alert, e.g., see “I feel OK to work.” Moreover, the second of the selectable user options 410 corresponds to an option to, at least temporarily, stop participating in the physical activity, e.g., see “I will rest.”

An indication may be received, from the user device, of a selection by the user of one of the selectable user options. For example, with reference now to FIG. 4B, it may be assumed that an indication of selection of the first selectable user options 412 has been received.

In response to receiving the indication of selection of the first selectable user option 412, it may be determined, after a predetermined period of time, whether the core body temperature of the user is greater than or equal to the predetermined threshold temperature. In response to a determination that the core body temperature of the user is not greater than or equal to the predetermined threshold temperature after the predetermined period of time, an alert 422 may be output for display on the user device that the user is ready to return to work. In the current approach, the alert 422 includes information 424 detailing why the user is allowed to return to work. A plurality of selectable user options 426, 428 may be output with the alert 422 for display on the display 402 to ensure that the user does not return to work prematurely. For example, the first selectable user option 426 corresponds to an option to return to work, e.g., see “I will return to work.” Moreover, the second of the selectable user options 428 corresponds to an option to, at least temporarily, continue stopped participating in the physical activity, e.g., see “I need more rest.”

In contrast to state 420 of FIG. 4B, referring now to state 440 of FIG. 4C, in response to a determination that the core body temperature of the user is greater than or equal to the predetermined threshold temperature after the predetermined period of time, an alert 442 is output for display on the user device. The alert 442 includes an instruction 444 for display on the user device, instructing the user to stop participating in the physical activity. Information 446 of the alert 442 may in one approach include suggestions that may assist the user in lowering their core body temperature.

In some approaches, a second alert may be output to a second user device of a second user, e.g., a supervisor of the user at a work site. The second alert may indicate that the core body temperature of the user is still greater than the predetermined threshold temperature. In one approach, information 448 of the alert 442 may notify the user of the second alert being output.

It should be prefaced that a determination that the core body temperature of the user is greater than or equal to the predetermined threshold temperature after the predetermined period of time is preferably a matter of concern because it is likely that the user, in electing to continue participating in the activity despite the first alert, misjudged their need to stop participation in the activity to allow their body to lower. Accordingly, it may be noted that the state 440 does not include selectable options to continue working for the predetermined period of time, based on the risk that continuing to participate in the physical activity may result in injury and/or illness to the user. One or more predetermined operations may additionally and/or be performed in an effort to protect the wellbeing of the user, e.g., an audio alert, a visual alert, a vibration alert output to the sensor device, etc. Such optional operations may constitute a safety escalation path that is performed until it is determined that the core body temperature of the user is not greater than the predetermined threshold temperature.

Various embodiments and approaches described herein may utilize one or more algorithms that are based on each user's baseline survey data, real-time physiological data collected from a device, environmental-based data of the user, clothing based data of the user and the user's interactions with the alerts. These data may be constantly evaluated while the individual is wearing the device, and algorithm may adapt over time to more closely follow and update user work/rest schedules according to new physiological data collected and/or user interactions with output alerts.

In some approaches, there may be two phases to this process in developing the individualized work/rest schedules. Initially, operations may be based on a user's real-time physiological data, the user's baseline survey data, environmental-based data of the user and clothing based data of the user to determine when to output “Stop Work” and “Ready to Work” alerts for display. The second phase is more complex and results in the creation of individualized work/rest schedules, where one or more operations may be based on the user's interactions when given an alert and/or the user's real-time physiological data, to update the schedules to become more personalized and accurate for each person, e.g., accurate in that no injuries and/or illness occur while participation in the physical activity is maximized.

Depending on the approach, code associated with such operations may be implemented on the device itself, on a connected remote host processor, on a cloud based data backend server, etc.

The individualized work/rest schedules are generated and updated over time to develop accuracies for each user that increase the longer that the user wears the sensor device. For example, the user may wear the sensor device on their arm during work. Baseline health data of the user may be received, e.g., medical history, anthropometric data, age, etc. Stop and ready to work alerts that are based on operations described herein are output to the user. Such alerts are based on the baseline health data and the activity-based health data of the user among other data, e.g., such as the environmental-based data of the user and/or clothing data of the user. User interaction with such alerts, e.g., indication of a selection by the user of selectable user options, may be considered to determine whether the stop and ready to work alerts are accurate, e.g., the alerts were output at a time that results in the user not experiencing illness and/or injury. Subject demographic data and changes in physiological data and environmental data may be logged. As a result, alerts output to the user thereafter become more accurate. This process may continue and as a result the operations become more accurate over time as adjustments are made for the user's fitness and acclimatization changes, adjustments are made for different environmental working conditions and adjustments are made as the user's clothing type changes throughout the year and/or it is determined that the user is participating in or planning to participate in a specific job function or tasks that a predetermined type of clothing is associated with, e.g., such as determined based on the received data.

Real-Time Work/Rest Schedules

Initially, when signing up for an account, a user may be asked to fill out a medical history questionnaire, e.g., age, weight, height, biological sex, etc. This information, along with the real-time physiological data being collected from the device while the user is wearing it, e.g., ambient temperature and humidity, skin temperature and humidity, heart rate and activity, etc., may be used to generate a “Stop Work” alert when the worker's core body temperature is detected to be at an unsafe level, e.g., about 38.2 degree Celsius. The user can then interact with the alert via their user device, e.g., elect to continue to work or elect to rest. This information is stored, and operations may thereby improve, e.g., become more accurate for a specific user, by accounting for each user's real-time physiological data, environmental data, and their selections when interacting with the “Stop Work” and “Ready to Work” alerts. In this way, operations describe herein may incorporate updated data for each user over time, e.g., deep learning, the work/rest schedules thereby become more accurate for that specific user over time. Additionally, as the worker acclimatizes to the heat, their threshold for the Stop Work alert will be raised to a core body temperature of 38.5 degree Celsius, e.g., which will describe elsewhere herein.

Predictive Work/Rest Schedules

After the user wears the sensor device for about a month, e.g., daily at work each day, based on the user interactions with the alert system, one or more operations may include outputting predictive work/rest schedules to each user in the morning and/or before a work shift. Accordingly, each morning, inputs that include the predicted ambient temperature and humidity in the user's planned location that day, e.g., input via a weather service, may be received. An estimate of the user's work/rest schedule that is unique to the user may be output to the user so that the user can plan their workday accordingly. The user's work/rest schedule may additionally and/or alternatively be output to the user's manager. This may enable the manager to make adjustments to work location, work time of day, workload, etc.

A predictive work/rest schedule and real-time work/rest schedules may be output in tandem each day. For example, the user may receive stop and ready to work alerts, and the user may decide whether to utilize the predictive work/rest feature. Together, features enabled by operations described herein offer techniques that are specific to individual users, to help to keep the users safe throughout the day while participating in physical activity in environmental conditions that are capable of causing injury and/or illness to the user, e.g., heat.

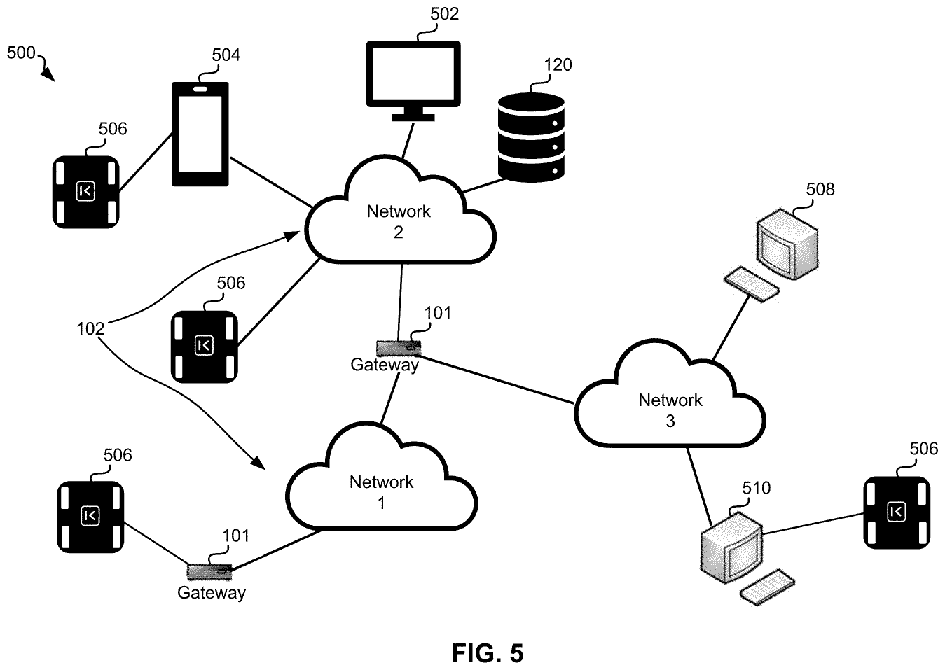

FIG. 5 depicts an architecture 500, in accordance with one embodiment. As an option, the present architecture 500 may be implemented in conjunction with features from any other embodiment listed herein, such as those described with reference to the other FIGS. Of course, however, such architecture 500 and others presented herein may be used in various applications and/or in permutations which may or may not be specifically described in the illustrative embodiments listed herein. Further, the architecture 500 presented herein may be used in any desired environment.

As shown in FIG. 5, a plurality of remote networks 102 are provided including a first remote network, e.g., see Network 1, and a second remote network, e.g., see Network 2. A gateway 101 may be coupled between the remote networks and a proximate network, e.g., see Network 3. In the context of the present network architecture 500, the networks 104, 106 may each take any form including, but not limited to a LAN, a WAN such as the Internet, public switched telephone network (PSTN), internal telephone network, etc.

A peripheral 120 or series of peripherals 120, e.g., facsimile machines, printers, networked storage units, etc., may be coupled to one or more of the networks. It should be noted that databases, servers, and/or additional components may be utilized with, or integrated into, any type of network element coupled to the networks. In the context of the present description, a network element may refer to any component of a network.

The second network is in communication with a display device 502, a wearable computing device 504 and a mobile computing device 506 worn by a user while participating in physical activity. Note that the wearable computing device 504 is also in communication with one of the plurality of mobile computing devices 506.

The first network is in communication with the gateway 101. The gateway 101 is in communication with one of the plurality of mobile computing devices 506.

The third network is in communication with a first computer 508 and a second computer 510. The second computer 510 is in communication with one of the plurality of mobile computing devices 506.

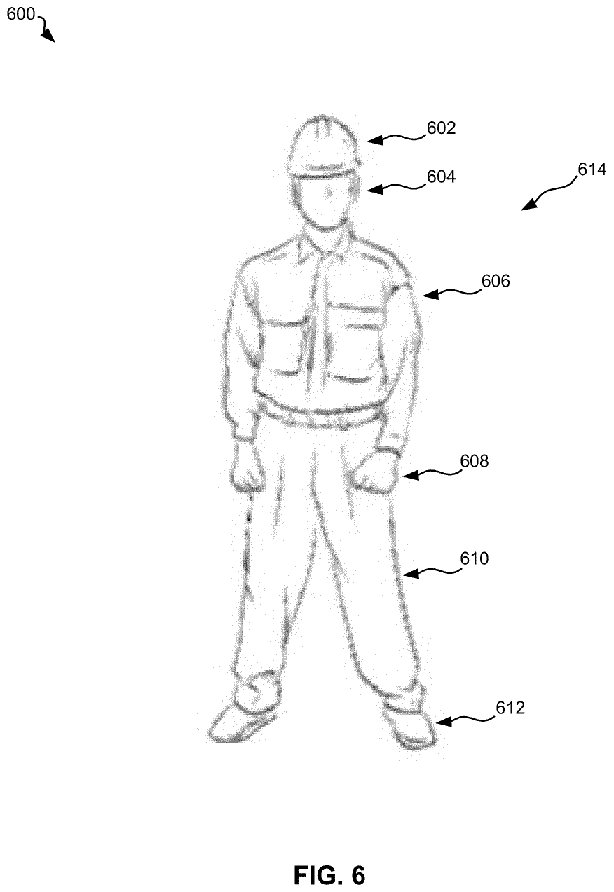

FIG. 6 depicts an illustration 600, in accordance with one embodiment. As an option, the present illustration 600 may be implemented in conjunction with features from any other embodiment listed herein, such as those described with reference to the other FIGS. Of course, however, such illustration 600 and others presented herein may be used in various applications and/or in permutations which may or may not be specifically described in the illustrative embodiments listed herein. Further, the illustration 600 presented herein may be used in any desired environment.

The illustration 600 of a user 614 wearing various forms of PPE. Data based on such PPE worn by the user and/or PPE expected to be worn by the user may be received and used for generating a personalized schedule for the user to follow while participating in the physical activity. For example, in one approach, such data may be received subsequent to outputting a questionnaire for the user to input one or more PPE layers that user is wearing and/or plans to wear within a predetermined period of time, e.g., during a scheduled work shift of the user, during a particular day of the week, in one or more anticipated weather patterns as specified in the questionnaire, etc. For purposes of an example, the user 614 is wearing: a hard hat 602, face/hearing protection 604, a protective shirt 606, hand coverings 608, protective leggings 610 and footwear 612. Of course, the extent and type of PPE worn by a user may depend on the approach.

Individualized Heat Acclimatization Program

As mentioned elsewhere herein, for each 1 degree Fahrenheit increase in summer temperatures, heat-related deaths may increase up to thirty-seven percent. Many of these heat-related problems result from users pushing too hard in the heat because the user (or their managers) do not know when they need to stop working and take a break.

Additionally, worksite environments, e.g., such as construction worksites, and athletic environments, e.g., such as an American football practice field, report that a majority of heat injuries and illnesses occur within the first few days that a user is present on the job, out on the field, etc. This is because users are not heat acclimatized, or accustomed to working in that environment, e.g., the user's body has not adapted to working in that climate.

Heat acclimatization is a process of repeated heat exposure which induces physiological changes that confer improved heat tolerance to an individual. For example, in order to prevent injury and/or illness, upon entering an environment to participate in a physical activity, a new user, e.g., such as a new worker entering a work environment, may be allowed a period of time to heat acclimatize upon starting to work in the heat, when re-entering into a hot environment, and/or when seasons change, e.g., the transition from winter to spring, the transition from spring to summer, etc. A majority of conventional techniques for heat acclimatization are based on one or more predetermined rules that only consider a limited number of variables among a broad spectrum and/or entire population of users. This is because such conventional techniques for heat acclimatization only consider a limited number of variables among workers, and ultimately do not account for the large physiological variability among individuals, e.g., biological sex, age, fitness level, acclimatization status, whether the worker is taking medication, what type and/or dosage of medication a worker is taking, etc. Moreover, conventional techniques for heat acclimatization are often designed from laboratory tests that utilize subject populations predominantly comprised of young, healthy men. Accordingly, typical heat acclimatization techniques are not accurate for women and older individuals, e.g., such as women or individuals over thirty-five years old. This does not ensure all workers equally heat acclimatize and as a result, users continue to be susceptible to heat-related injuries.

In sharp contrast to the heat acclimatization deficiencies described above, various embodiments and approaches described herein ensure that a user is properly heat acclimatized before participating in a physical activity by determining whether a core body temperature of a user has increased a predetermined amount from the baseline core body temperature of the user for a predetermined amount of time during each of a predetermined number of time intervals.

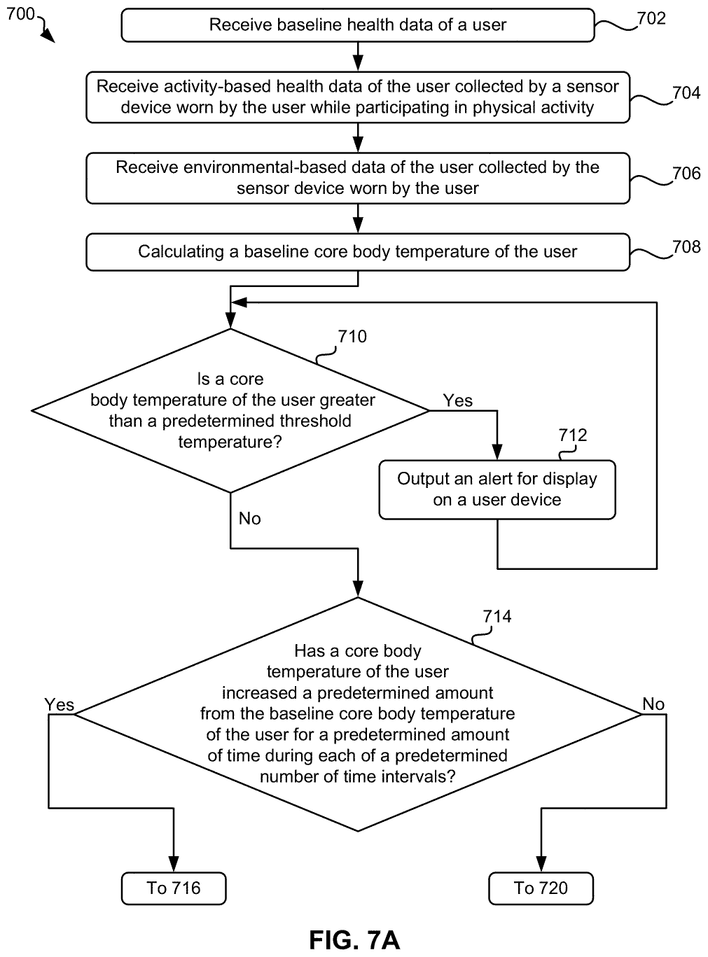

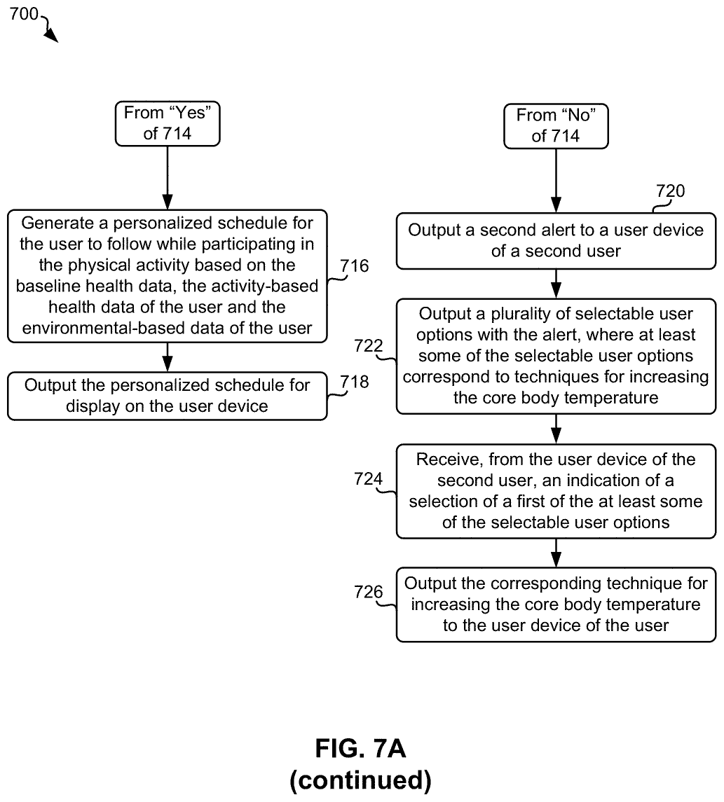

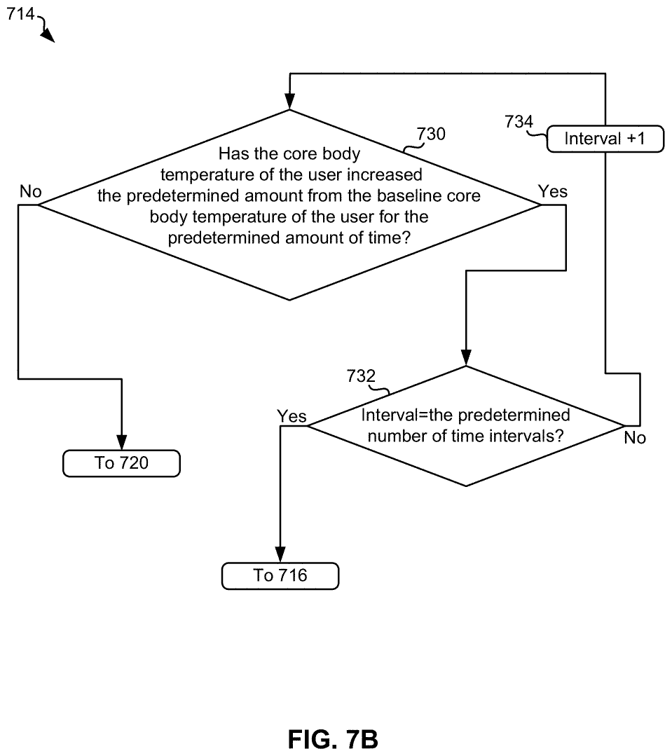

FIG. 7A shows a method 700, in accordance with one embodiment. As an option, the present method 700 may be implemented in devices such as those shown in the other FIGS. described herein. Of course, however, this method 700 and others presented herein may be used to provide applications which may or may not be related to the illustrative embodiments listed herein. Further, the methods presented herein may be carried out in any desired environment. Moreover, more or less operations than those shown in FIG. 7A may be included in method 700, according to various embodiments. It should also be noted that any of the aforementioned features may be used in any of the embodiments described in accordance with the various methods.

It may be prefaced that method 700 may in some approaches be initiated in response to detecting that one or more trigger events have occurred. A non-limiting list of such trigger events may include, e.g., automatically at the creation of a new user account, user selection on an application or user device such as to “start” the user's individualized heat acclimatization process, a prompt via an application and/or tablet based on a user's medical or work history, a manager selection for a specific new worker (or de-acclimatized worker) that the manager deems should be heat acclimatized, via a prompt to the user to initiate the program as seasons change such as from winter to summer, via a prompt to the user to initiate the program as a sudden heat wave is incoming to a geographical location of the user, via a prompt based on a detection of a worker's loss of heat acclimatization status, etc.

It may also be prefaced that although operations of methods described herein may be described being performed by one component of a system, in some approaches each of such operations may be performed by any number of components, e.g., in a scaled-out approach where each of such components are executing a method.

Operation 702 includes receiving baseline health data of a user. Note that the received baseline health data may be pre-acquired, e.g., using a known type of health data input module, using a known technique for gathering health data from one or more sources, based on one or more questionnaires, processing images of a user using one or more known types of recognition techniques, etc. A non-limiting list of the baseline health data of the user may include, e.g., medical history data, anthropometric data such as weight and/or height, age, biological sex, a determined body mass index, etc.

Activity-based health data of the user, e.g., biometric data, collected by a sensor device worn by the user while participating in physical activity is received, e.g., see operation 704 of method 700. Accordingly, in some approaches, the sensor device may be configured to continually, e.g., according to a predetermined interval, and non-invasively, collect predetermined metrics of the user using one or more known types of sensors, e.g., a camera, a heartbeat sensor, a temperature sensor for determining a temperature of the user, a humidity sensor, a motion sensor, a global positioning system, a proximity sensor, a gyroscope, an accelerometer, a microphone, a heat flux sensor, etc. The activity-based health data may depend on the approach. For example, in some approaches, the activity-based health data may include physiological data of the user, e.g., ambient temperature, ambient humidity, skin temperature of the user, skin humidity of the user measured by the sensor device, near skin humidity of the sensor, a perspiration rate of the user, a heart rate of the user such as measured via photoplethysmography or via one or more known techniques, an activity type that the user is determined to be engaged in and data associated therewith, a rate of movement of the user, etc.

It should be noted that the type of physical activity may depend on the approach. For example, because in some approaches the activity-based health data of the user is collected and/or received prior to a determination that the user has been heat acclimatized, the activity-based health data of the user may be based on a limited activity schedule of the user, e.g., such while the user is following a personalized schedule for participating in the physical activity that includes only task assignments that are predetermined to not likely be capable of raising the core body temperature of the user. In another example, in some approaches the activity-based health data of the user may be collected while the user is participating in orientation-based physical activity, e.g., where the user may be allowed to fully participate in the physical activity subsequent to it being determined that the user is heat acclimatized. Note that various techniques for determining whether the user has reached heat acclimatization are described elsewhere herein, e.g., see method 800. In another approach, the activity-based health data of the user may additionally and/or alternatively be collected while the user is performing nominal daily tasks, e.g., awake in the user's home, driving to work, attending school, watching a user device, sitting stationary, etc.

Operation 706 includes receiving environmental-based data of the user collected by the sensor device worn by the user. Data based on clothing worn by the user and/or clothing expected to be worn by the user may additionally and/or alternatively be received in an optional operation of method 700. The environmental-based data and/or the clothing-based data may be similar to and/or collected using similar techniques to the environmental-based data and/or the clothing-based data described elsewhere herein, e.g., see operation 305 of method 300.

With continued reference to FIG. 7A, a baseline core body temperature of the user is calculated, e.g., see operation 708. In some preferred approaches, operation 708 may additionally and/or alternatively include ongoingly calculating the core body temperature of the user, e.g., continuously, which may be used in the alerts described elsewhere below. For context, and as will be described in further detail elsewhere below, e.g., see decision 714 of method 700, the baseline core body temperature of the user may be used as a reference point that is used to determine whether the user's body is safely becoming acclimatized to heat.

The core body temperature of the user may be calculated at any time. In one preferred approach, the core body temperature may be defined as the core body temperature of the user subsequent to the user being registered to be heat acclimatized, but prior to the user altering their amount of user activity more than a predefined amount from previous sample of the user's activity. In some other approaches, the baseline core body temperature of the user may be calculated using user data collected over a predetermined period of time, regardless of the user's user activity within the predetermined period time.

One or more types of user data may be used to calculate the baseline core body temperature of the user. In some approaches, the baseline core body temperature of the user may be calculated using general linear regression models that predict core temperature using physiological sensor data, e.g., such as the activity-based health data of the user. In yet another approach, the baseline core body temperature may be an average of the user's core body temperature sampled a predetermined number of times over a predetermined number of time intervals, e.g., one core body temperature sample a day for two days, three core body temperature samples a day for one week, fifty core body temperature samples a day for ten days, etc. The baseline core body temperature of the user may additionally and/or alternatively be determined using a table that includes a plurality baseline core body temperature values that are pre-associated to values of the received data. The baseline core body temperature of the user may additionally and/or alternatively be calculated using data modeling techniques. In one or more of such approaches a database may be generated by the data modeling using ongoingly collected and/or updated values of the received data. Data of the database may be comparatively analyzed with the received data of the user in order to determine a current average baseline core body temperature of the user. Test case research and extrapolation based on results may additionally and/or alternatively be used for calculating the baseline core body temperature of the user. For example, in some approaches, the received data of the user may be applied to one or more known techniques of test case research and extrapolation to determine the baseline core body temperature of the user. One or more known types of calculations may be additionally and/or alternatively be used for calculating the baseline core body temperature of the user. For example, in some approaches, the received data of the user may be applied to a black box equation having an output that includes a baseline core body temperature of the user. In another approach, the received data of the user may be applied to a known type of machine learning algorithm to generate and/or ongoingly update the baseline core body temperature of the user.

Method 700 may include one or more optional operations to ensure that a user that is in the process of being heat acclimatized does not experience injury and/or illness as a result of the core body temperature of the user exceeding a safe core body temperature. For example, in order to prevent the user from continuing to participate in the physical activity to an extent that would otherwise result in bodily harm to the user, it may be determined whether the core body temperature of the user greater than a predetermined threshold temperature and/or a rate of change in core body temperature of the user exceeds a predetermined “safe” rate, e.g., see decision 710 of method 300. Note that the predetermined threshold temperature may be any temperature, and may be set and/or adjusted by one or more input sources, e.g., a manager of the user, an employer of the user, a doctor of the user, a global and/or national health committee, the user, etc. In some preferred approaches, the predetermined threshold temperature is in a range of about 37.8 degrees Celsius to about 38.6 degrees Celsius. However, the predetermined threshold temperature may be set and/or adjusted outside of this range.

For context, it should be noted that in some approaches, excessive exposure to heat, or the user having a core body temp exceeding thirty-nine degrees Celsius may be detrimental to the user's health and slow the heat acclimatization process, e.g., due to missed work or heat injuries and illnesses. Accordingly, in some approaches, in response to a determination that the core body temperature of the user is greater than the predetermined threshold temperature (e.g., as illustrated by the “Yes” logical path of decision 710) and/or in response to a determination that the rate of change in core body temperature of the user exceeds a predetermined safe rate, the user may be notified and/or alerted to the relatively unsafe user condition. For context, the user may be notified and/or alerted to the relatively unsafe user condition in order to enable the user to recover, e.g., lower the core body temperature of the user, before potentially experiencing an injury and/or illness as a result continuing to participate in the physical activity while having a core body temperature of the user is greater than the predetermined threshold temperature. Such an alert may assist the user in avoiding injury and/or illness because many users that are injured and/or become ill as a result of participating in a physical activity do not realize that they should stop to lower their core body temperature until it is too late to do so, e.g., until injury and/or illness is unavoidable. An alert is in some approaches output for display on the user device in response to a determination that a core body temperature of the user is greater than a predetermined threshold temperature, e.g., see operation 712 of method 300. The alert may include one or more instructions and/or information similar to the alert described elsewhere herein, e.g., see operation 308 of method 300. Accordingly, it should be noted that the alert may include a warning, e.g., a “danger” alert, in order to ensure the safety of the user while heat acclimatizing. As illustrated by the logic of operation 712 returning to decision 710, in some approaches, continuing method 700 may depend on the core body temperature of the user returning to a safe temperature, e.g., less than or equal to the predetermined threshold temperature. It should also be noted that additional alerts, e.g., similar to operation 316 and/or operation 318 of method 300, may be output in response to a determination that the core body temperature of the user continues to be greater than the predetermined threshold temperature. For example, in one approach, a second alert may be output to the user device of the user and/or a user device of a second user that is in a position to assist the user in lowering their core body temperature, e.g., a supervisor of the user, a coach of the user, a medical professional, a family member of the user etc.

In contrast, in response to a determination that the core body temperature of the user is not greater than the predetermined threshold temperature (e.g., as illustrated by the “No” logical path of decision 710) and/or in response to a determination that the rate of change in core body temperature of the user does not exceed a predetermined safe rate, method 700 optionally continues to decision 714.

Decision 714 includes determining whether a core body temperature has increased a predetermined amount from the baseline core body temperature of the user for a predetermined amount of time during each of a predetermined number of time intervals. Consideration of changes of the user's core body temperature from the user's baseline core body temperature during each of the predetermined number of time intervals is important because heat acclimatization of the user may be unlikely to be achieved based merely on an initial instance of the user's core body temperature increasing from the baseline core body temperature. This is because a determination that heat acclimatization has been achieved by the user may be based on several samples being considered. Note that consideration of only one sample of the user's core body temperature may be inaccurate and cause injury and/or illness of the user if a determination that the user is heat acclimatized is made prematurely, e.g., based on a small data sample. Accordingly, various approaches described herein base heat acclimatization on the user's core body temperature across the predetermined number of time intervals.

According to some approaches, the predetermined amount of temperature increase may be in a range of about one degree Celsius to about three degrees Celsius. In one preferred approach, the predetermined amount of temperature increase may be about one degree Celsius, e.g., the predetermined amount of temperature increase may be calculated to be one degree Celsius greater than the baseline core body temperature of the user. The predetermined amount of temperature increase may be set and/or adjusted by one or more users, e.g., a manager of the user, a medical professional that the user sees, a coach of the user, medical organization guidelines, the user, etc. Moreover, the predetermined amount of temperature increase may be adjusted in response to detecting one or more trigger events, e.g., the predetermined amount of temperature increase may be decreased in response to a determination that the rate of change in core body temperature of the user exceeds a predetermined safe rate, the predetermined amount of temperature increase may be increased in response to a determination that the user has experienced a heat related injury and/or illness, the predetermined amount of temperature increase may be increased in response to a determination that an average temperature of a geographical location where the user is present has increased, the predetermined amount of temperature increase may be decreased in response to a determination that an average temperature of a geographical location where the user is present has decreased, based on a weather forecast, etc.

The predetermined amount of time may in some approaches be in a range of thirty minutes to two hours. According to one preferred approach, the predetermined amount of time may be one hour. Moreover, each of the time intervals may in some approaches be about twenty-four hours, e.g., one day, and the predetermined number of time intervals may be about two to thirty days. According to one preferred approach, the predetermined amount of time may be one hour, and the predetermined number of time intervals may be three to fourteen, e.g., three to fourteen days where each of the intervals are defined to be about twenty-four hours. In such an approach, assuming that the user has a baseline core body temperature of 37.2 degrees Celsius, decision 714 may include determining whether the user's core body temperature has increased the predetermined amount of temperature increase from the user's baseline core body temperature of 37.2 degrees Celsius for at least one hour per day for three to fourteen days. Note however, that the above ranges are provided for purposes of illustritive examples only. Accordingly, in some other approaches, values described in various approaches described herein, e.g., the predetermined amount of time, the predetermined number of time intervals, the predetermined amount of temperature increase, etc., may fall outside of the ranges disclosed herein.

In some approaches, the predetermined amount of time may be a series of increasing values that are each assigned to a different one of the time intervals. For example, method 700 may include determining whether the core body temperature has increased the predetermined amount from the baseline core body temperature of the user for thirty minutes during a first of the time intervals, determining whether the core body temperature has increased the predetermined amount from the baseline core body temperature of the user for one hour during a second of the time intervals and determining whether the core body temperature has increased the predetermined amount from the baseline core body temperature of the user for one hour and one-half hour during a third of the time intervals. Note that according to some other approaches, the predetermined amount of time and/or the predetermined amount that the user's core body temperature increases from the baseline core body temperature of the user may additionally and/or alternatively be, e.g., a series of increasing values, a series of decreasing values, a varying pattern of values, etc.

Looking to FIG. 7B, exemplary sub-processes of determining that the core body temperature of the user has increased the predetermined amount from the baseline core body temperature of the user for the predetermined amount of time an initial one of the predetermined number of time intervals are illustrated in accordance with one embodiment, one or more of which may be used to perform decision 714 of FIG. 7A. More specifically, the exemplary sub-processes of FIG. 7B is included to illustrate the series of determinations that may be performed across the intervals. However, it should be noted that the sub-processes of FIG. 7B are illustrated in accordance with one embodiment which is in no way intended to limit the invention.

It may be determined whether the core body temperature of the user has increased the predetermined amount from the baseline core body temperature of the user for the predetermined amount of time, e.g., see sub-operation 730. In some approaches, if in any of the one or more performed iterations it is determined that the core body temperature of the user has increased the predetermined amount from the baseline core body temperature of the user for the predetermined amount of time, the method may continue to operation 720, e.g., see the “No” logical path of sub-operation 730 return to operation 720. One or more alerts may be output to the user device of the user and/or a user device of a second user that indicate the determinations the outcome of sub-operation 730. In contrast, in response to determining that the core body temperature of the user has increased the predetermined amount from the baseline core body temperature of the user for the predetermined amount of time for a first interval, sub-operation 730 may be performed for the predetermined number of intervals, e.g., see sub-operation 732 and sub-operation 734. In response to determining that the core body temperature of the user has increased the predetermined amount from the baseline core body temperature of the user for the predetermined amount of time for each of the predetermined number of time intervals, e.g., as illustrated by the “Yes” logical path of sub-operation 732, method 700 optionally continues to operation 716.

With reference again to FIG. 7A, in some approaches, in response to determining that the core body temperature of the user has increased the predetermined amount from the baseline core body temperature of the user for the predetermined amount of time an initial one of the predetermined number of time intervals, the user and/or a second user, e.g., a manager of the user, may or may not be alerted that the user has reached a sufficient stimulus to induce heat acclimatization depending on the user's individual situation. Accordingly, in some approaches, such an alert may be output in accordance with one or more outputs to stop and/or to start participating in the physical activity, e.g., see method 300. Optionally incorporating the individualized heat acclimatization process with the alert system of method 300, enables the user to stay safe in the heat while achieving heat acclimatization. Together, these processes will help the worker to safely become more heat tolerant and fit for the job.

This process of ensuring that the user continues to have a core body temperature that has increased the predetermined amount from the baseline core body temperature of the user for the predetermined amount of time may in some approaches continue until it is determined that the user has achieved heat acclimatization. Note that various techniques for determining whether the user has reached heat acclimatization are described elsewhere herein, e.g., see method 800. As each individual user is genetically different, it may take varying lengths of time for users to heat acclimatize. During this process, the alerts and notifications described herein may guide the user until it is determined that the user is heat acclimatized. Once this determination is made, an alert may be output to the user device of the user. Note that some users may be prepared to fully participate in the physical activity in as soon as the fourth interval of method 400, e.g., a fourth day of increased core body temperature, while others may take additional time to acclimatize, e.g., up to a fourteenth day of increased core body temperature or more.

For context, it should also be noted that heat acclimatization and/or a user's process toward heat acclimatization can be lost relatively rapidly, e.g., within 5-7 days, in the absence of heat stress. Accordingly, it may be important to alert the user and/or a second user in the event that it is determined that the user has lost their heat acclimatization status, and need to re-acclimatize. The re-acclimatization process may be a relatively faster process than the initial heat acclimatization process, e.g., 2-3 days, as will be described in greater detail elsewhere herein, e.g., see method 800.

In response to a determination that the core body temperature has increased the predetermined amount from the baseline core body temperature of the user for the predetermined amount of time during each of the predetermined number of time intervals (as illustrated by the “Yes” logical path of decision 714), a personalized schedule may be generated for the user to follow while participating in the physical activity, e.g., see operation 716. The personalized schedule may be based on the baseline health data of the user, the activity-based health data of the user, clothing based data and/or the environmental-based data of the user. The user may be determined to be heat acclimatized in response to the determination that the core body temperature has increased the predetermined amount from the baseline core body temperature of the user for the predetermined amount of time during each of the predetermined number of time intervals. Accordingly, in some approaches, the generated personalized schedule may include instructions for a relatively greater amount of participation in the physical activity than an amount of participation in the physical activity that the user was limited to prior to determining that the user has successfully heat acclimatized. Note that optionally limiting the user's participation in the physical activity, e.g., based on an assignment and/or an instruction output to the user device, prior to it being determined that the user has heat acclimatized may minimize heat injuries and/or illnesses and to allow the user sufficient time to adjust to environmental conditions that the user is present in. In one or more approaches, the generated personalized schedule may include instructions for a predetermined amount of participation in the physical activity, e.g., where the predetermined amount is a predetermined multiple of the amount of participation in the physical activity that the user was limited to prior to determining that the user has successfully heat acclimatized, where a plurality of instructions of the generated personalized schedule ramp up participation in the physical activity according to a predefined rate, etc.

When followed by the user, the personalized schedule may help prevent the user from having a core body temperature that exceeds a predetermined safe core body temperature. In some approaches, the personalized schedule may be similar in type and generated using similar techniques as other personalized schedules described elsewhere herein, e.g., see operation 320 of method 300. Moreover, the generated personalized schedule may be modified using techniques similar to those described elsewhere herein, e.g., trade requests, modifications initiated by a second user, etc.

With continued reference to FIG. 7A, the personalized schedule is output for display on the user device, e.g., see operation 718 of method 700.

In one optional approach, the user may be alerted of their progress during the process of heat acclimatization. For example, in some approaches, an alert may be generated and output each interval that it is determined that the user's core body temperature successfully increases the predetermined amount from the baseline core body temperature of the user for the predetermined amount of time. Accordingly, a daily “alert” or notification, which may be output via any one or more of the means described elsewhere herein, may be ongoingly output to the user device provided that the user has successfully reached the time & temperature threshold needed to induce heat acclimatization that day.

In response to a determination that the core body temperature has not increased the predetermined amount from the baseline core body temperature of the user for the predetermined amount of time during each of the predetermined number of time intervals (as illustrated by the “No” logical path of decision 714), an alert may be output to a user device of a second user, e.g., see operation 720. Alerts may be output via, e.g., SMS, a tablet, a phone app, a desktop app, dashboard, etc. The second user may in some approaches be an authoritative figure and/or someone with the ability to assist the user in participating in the physical activity in a manner that results in the core body temperature of the user increasing the predetermined amount from the baseline core body temperature of the user for the predetermined amount of time during each of the predetermined number of time intervals. For example, in some approaches, the second user may be, e.g., a supervisor of the user at a work site, a doctor of the user, a family member of the user, someone that the user has designated as an emergency contact on medical forms, a co-worker that has experience with a line of work that the user performs such that the second user can cover for the user while the user at least temporarily modifies their participation in the physical activity, etc.

The alert may include one or more types of information that may vary in type depending on the approach. For example, in some approaches the alert includes a notification that the core body temperature of the has not increased the predetermined amount from the baseline core body temperature of the user for the predetermined amount of time during each of the predetermined number of time intervals.

A plurality of selectable user options may be output with the alert in some approaches, e.g., see operation 722. At least some of the selectable user options may correspond to techniques for increasing the core body temperature of the user. For example, at least some the selectable user options may be suggestions that the second user can pick from to help the increase core body temperature of the user the predetermined amount from the baseline core body temperature of the user for the predetermined amount of time during each of the predetermined number of time intervals. According to some more specific examples, the techniques for increasing the core body temperature may be based on a current geographical location of the user. In some approaches where the techniques for increasing the core body temperature are based on a current geographical location of the user, a non-exhaustive list of techniques for increasing the core body temperature of the user may include, e.g., selecting one or more relatively hotter geographical locations for the user to participate in the physical activity and/or merely rest stationary in, selecting one or more relatively more humid geographical locations for the user to participate in the physical activity and/or merely rest stationary in, selecting that the user move to a different geographical location based on API calls to a weather source and/or network connected environmental sensors, etc. In some other approaches, the techniques for increasing the core body temperature may be additionally and/or alternatively based on the physical activity that the user is participating in. For example, where the techniques for increasing the core body temperature of the user are based on the physical activity that the user is participating, a non-exhaustive list of techniques for increasing the core body temperature of the user may include, e.g., prescribing a relatively more rigorous physical activity for the user to participate in while the user is in the process of heat acclimatizing, increasing a time that the user is currently limited to participating in the physical activity while the user is in the process of heat acclimatizing, changing a work shift time of the user such as to a hotter part of the day, adding an additional physical activity for the user to participate in while the user is in the process of heat acclimatizing, etc. According to some other specific example, the techniques for increasing the core body temperature may be based on a clothing that the user is wearing and/or plans to wear (i.e. adding or removing more clothing layers). Accordingly, where one of more of the techniques for increasing the core body temperature of the user are based on clothing that the user is wearing and/or plans to wear, a non-exhaustive list of techniques for increasing the core body temperature of the user may include, e.g., suggesting a modification to clothing layers of the user throughout the heat acclimatization process such as day one wearing minimal clothing and day five wearing full clothing, suggesting a change in the shade of the user's clothing, suggesting a change in the clothing material of the user's clothing, etc. Such suggestions may maximize productivity at a job site, minimize injuries and illnesses, and remove doubt from managers as to whether users, and specifically each individual user, has sufficiently heat acclimatized and is safe to take on a full workload, e.g., fully participate in the physical activity.

An indication of a selection of one of the selectable user options may be received, e.g., from the user device of the second user. For example, operation 724 includes receiving, from the user device of the second user, an indication of a selection of a first of the at least some of the selectable user options. In some approaches, the indicated selection may be relayed to a user device of the user so that the user is provided with insight as to how to increase their core body temperature. Accordingly, operation 726 includes outputting the corresponding technique for increasing the core body temperature to the user device of the user.

At any time, subsequent to a determination that the core body temperature of the user has not increased the predetermined amount from the baseline core body temperature of the user for the predetermined amount of time during each of the predetermined number of time intervals, it may be again determined whether the user has been successfully heat acclimatized. For example, decision 714 may be repeated, e.g., after a predetermined amount of time has passed since it has been determined that the core body temperature of the user has not increased the predetermined amount from the baseline core body temperature of the user for the predetermined amount of time during each of the predetermined number of time intervals, subsequent to detecting that the user has performed the corresponding technique for increasing the core body temperature, in response to determining that environmental conditions of the geographical location that the user is present in have changed a predetermined amount, etc.

In the techniques of various approaches described above with respect to heat acclimatization, e.g., see method 700, it is important to note that user individualized heat acclimatization has heretofore not been considered and/or incorporated into conventional heat acclimatization efforts. As mentioned elsewhere above, this is because conventional techniques for heat acclimatization only consider a limited number of variables among workers, and ultimately do not account for the large physiological variability among individuals, e.g., biological sex, age, fitness level, acclimatization status, whether the worker is taking medication, what type and/or dosage of medication a worker is taking, etc. Moreover, conventional techniques for heat acclimatization are often designed from laboratory tests that utilize subject populations predominantly comprised of young, healthy men. Accordingly, typical heat acclimatization techniques are not accurate for women and older individuals, e.g., such as women and individuals more than thirty-five years old. This does not ensure all workers equally heat acclimatize and as a result, users continue to be susceptible to heat-related injuries. In sharp contrast to the acclimatization deficiencies described above, various embodiments and approaches described herein ensure that a user is properly heat acclimatized before participating in a physical activity by determining whether a core body temperature of a user has increased a predetermined amount from the baseline core body temperature of the user for a predetermined amount of time during each of a predetermined number of time intervals. Overall, the heat acclimatization techniques of various approaches described herein are optionally specific to each individual user and thereby ensure that each user is receiving sufficient stimulus to acclimatize to heat, while simultaneously preventing heat-related injuries and illnesses that typically occur within the first few days on of a user participating in a physical activity after returning from a hiatus, or as the result of a weather change. Accordingly, the inventive discoveries disclosed herein with regards to individualized heat acclimatization techniques proceed contrary to conventional wisdom.

It should further be noted that, as a result of ensuring that a core body temperature of a user has increased a predetermined amount from the baseline core body temperature of the user for a predetermined amount of time during each of a predetermined number of time intervals before the user is encouraged to fully participate in a physical activity, a significant number of injury events and illness events that would otherwise potentially occur as a result of users following general heat acclimatization techniques based on a limited sample of data will be mitigated. Note that these injuries and/or illnesses result in additional and resource-intensive processing tasks in computer systems associated with the conventional techniques, as adjustments to avoid such injuries and/or illnesses are ongoingly calculated. In sharp contrast, performance of computer systems are improved as a result of the techniques of various embodiments and approaches described herein enabling increased heat acclimatization accuracies by performing user individualized heat acclimatization processes.

Initial detection of heat acclimatization of an individual user and individualized heat re-acclimatization subsequent to a determination that a user has become heat de-acclimatized

As mentioned elsewhere herein, for each 1 degree Fahrenheit increase in summer temperatures, heat-related deaths may increase up to thirty-seven percent. Many of these heat-related problems result from users pushing too hard in the heat because the user (or their managers) do not know when they need to stop working and take a break. Additionally, worksite environments, e.g., such as construction worksites, and athletic environments, e.g., such as an American football practice field, report that a majority of heat injuries and illnesses occur within the first few days that a user is present on the job, out on the field, etc. This is because users are not heat acclimatized, or accustomed to working in that environment, e.g., the user's body has not adapted to working in that climate.

Heat acclimatization is a process of repeated heat exposure which induces physiological changes that confer improved heat tolerance to a user. For example, in order to prevent injury and/or illness, upon entering an environment to participate in a physical activity, a new user, e.g., such as a new worker entering a work environment, may be allowed a period of time to heat acclimatize upon starting to work in the heat, when re-entering into a hot environment, and/or when seasons change, e.g., the transition from winter to spring, the transition from spring to summer, etc. A majority of conventional techniques for heat acclimatization are based on one or more predetermined rules that only consider a limited number of variables among a broad spectrum and/or entire population of users. This is because such conventional techniques for heat acclimatization only consider a limited number of variables among workers, and ultimately do not account for the large physiological variability among individuals, e.g., biological sex, age, fitness level, acclimatization status, whether the worker is taking medication, what type and/or dosage of medication a worker is taking, etc. Moreover, conventional techniques for heat acclimatization are often designed from laboratory tests that utilize subject populations predominantly comprised of young, healthy men. Accordingly, conventional heat acclimatization techniques are not accurate for women and older individuals, e.g., such as women more than thirty-five years old. This does not ensure all workers equally heat acclimatize and as a result, users continue to be susceptible to heat-related injuries. Moreover, because conventional heat acclimatization techniques are based on a limited number of variables among a broad spectrum and/or entire population of users, it is not considered whether users that have achieved heat acclimatization thereafter lose heat acclimatization.

In sharp contrast to the heat acclimatization deficiencies described above, various approaches described herein ensure that a user maintains heat acclimatization in order to prevent the user from experiencing injury and/or illness as a result of otherwise being in an environment without being heat acclimatized to the environment. For example, in various approaches described herein, in response to a determination that the user has been de-acclimatized to an environment, the user and/or another user may be notified and a process for heat re-acclimatization may be initiated.

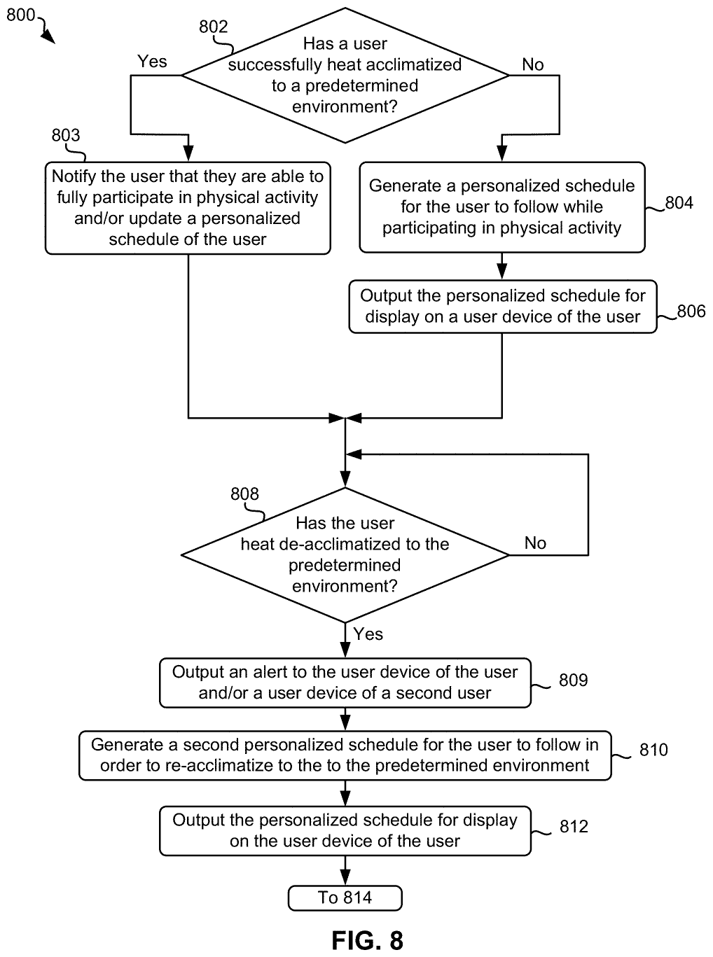

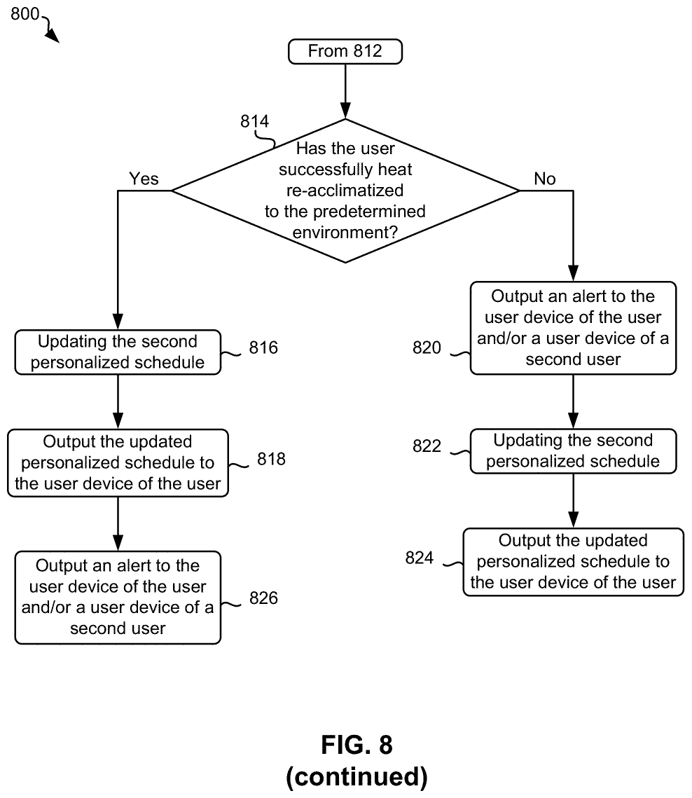

FIG. 8 shows a method 800, in accordance with one embodiment. As an option, the present method 800 may be implemented in devices such as those shown in the other FIGS. described herein. Of course, however, this method 800 and others presented herein may be used to provide applications which may or may not be related to the illustrative embodiments listed herein. Further, the methods presented herein may be carried out in any desired environment. Moreover, more or less operations than those shown in FIG. 8 may be included in method 800, according to various embodiments. It should also be noted that any of the aforementioned features may be used in any of the embodiments described in accordance with the various methods.

For context, it should be prefaced that method 800 may be related to method 700 in that, as described in greater detail elsewhere above, various techniques of method 700 enable a user to reach heat acclimatization and, as described in greater detail elsewhere below, method 800 enables a user to be provided with electronic information that details a detection of the user becoming fully heat acclimatized, and information that enables the user to retain and/or regain heat acclimatization subsequent to losing heat acclimatization. Because users typically heat acclimatize at different rates, e.g., such as anywhere from 3-14 days, it may be important that a user and/or a manager of the user is notified when a heat acclimatization process is complete, e.g., so that the user can fully participate in a physical activity such as assuming a full workload. Note however, that the user may still heed to alerts to start participating in physical activity and/or alerts to stop participating in physical activity, e.g., see method 300 and/or method 700. Heat acclimatization can be lost relatively quickly if a worker is not exposed to heat stress, e.g., shift work, vacation, seasonal changes in weather, illness, job change, etc. Various approaches described herein detect for a user becoming heat de-acclimatized, at which point, the user may be alerted that they have lost their heat acclimatization status and need to re-acclimatize. More specifically, various approaches described herein monitor for and detect several physiological changes that may occur with heat acclimatization and/or heat de-acclimatization to determine whether the user has acclimatized to heat or lost heat acclimatization status. In some approaches described elsewhere below, upon making such determinations, a user device of the user and/or a user device of a second user may be output a notification of the determined acclimatization status.

It may additionally be prefaced that one or more operations of method 800 and/or operations of other approaches described herein may incorporate information, e.g., such as variables of health data, that may be monitored and/or recorded using a sensor device, e.g., such as the sensor device described elsewhere herein. For example, the sensor device may include a patch component, or any other equivalent physiological monitoring device, that the user wears while heat acclimatizing to an environment, e.g., see method 700. During this time, the user's physiological data, e.g., via the sensors on the sensor device, may be continuously monitored. For example, in some approaches, these variables may include heart rate, sweat rate, skin temperature, ambient temperature, activity, core body temperature, etc. As will be described below, such user data may be combined with user and/or manager inputs via a dashboard and/or application and/or with weather data, e.g., via an API call, to detect when a worker has gained or lost heat acclimatization, e.g., see decision 802 and/or decision 808 of method 800.

Decision 802 of method 800 includes determining whether a user has successfully heat acclimatized to a predetermined environment, e.g., initially heat acclimatized to the predetermined environment. In some approaches, determining whether a user has successfully heat acclimatized to a predetermined environment may include determining whether a core body temperature of the user has increased a predetermined amount from a baseline core body temperature of the user for a predetermined amount of time during each of a predetermined number of time intervals. According to some more specific approaches, each of the time intervals may be about twenty-four hours and the predetermined number of time intervals may be about two to thirty. In some approaches, the determination of whether the user has successfully heat acclimatized to the predetermined environment may additionally and/or alternatively be performed using techniques described elsewhere herein, e.g., see decision 714 of method 700. The predetermined environment may depend on the approach. In some preferred approaches, the predetermined environment is an environment that the user is currently in and/or plans to be at. A non-limiting list of predetermined environments includes, e.g., a geographical location of the user, an environment where the user has spent at least some time during a heat acclimatization process, a location at which a user is determined to spend a majority of their time, an environment that the user plans to participate in physical activity, etc.

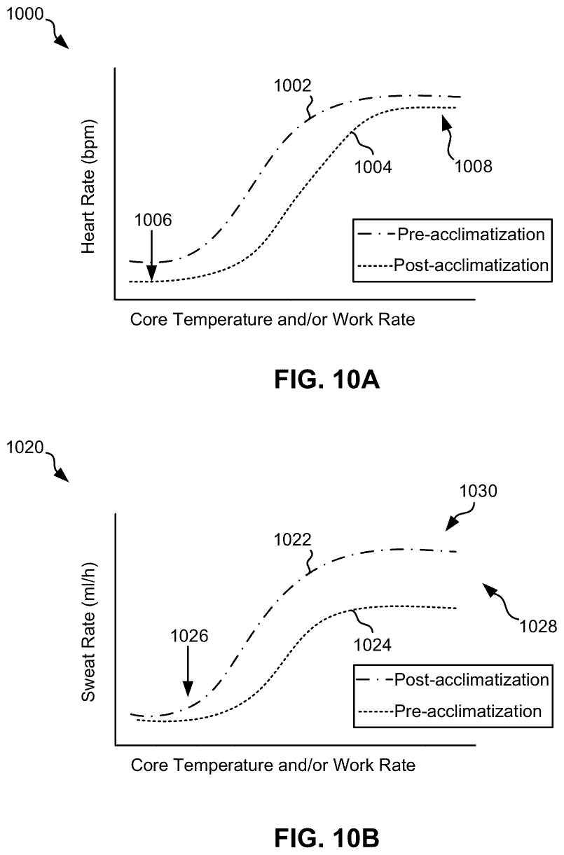

As indicated elsewhere above, user data such as physiological variables, e.g., such as heart rate, sweat rate, core body temperature, other physiological changes, other environmental changes, etc., may be collected and/or received. In some approaches this data may be collected while the user is at rest and while the user is participating in a physical activity, e.g., in the heat, to determine signature changes in each individual associated with heat acclimatization. For example, method 800 may include creating a baseline, e.g., baseline user data, in order to detect when the following physiological changes occur during a heat acclimatization process. Note that various of such changes are illustrated elsewhere herein for purposes of an example, e.g., see FIGS. 10A-10B.

According to further approaches, such physiological changes may be detected as trigger events to determine whether the user has successfully heat acclimatized to the predetermined environment. For example, in some approaches, the determination of whether the user has successfully heat acclimatized to the predetermined environment may be based on a detection of trigger events. A non-limiting list of such trigger events may include, e.g., the user having a relatively decreased core temperature during a period in which the user is not participating in a predetermined physical activity than a period in which the user is participating in the predetermined physical activity such as during a rest period (e.g., relative to the core body temperature of the user while participating in a physical activity); the user having a relatively lower heart rate during the period in which the user is not participating in a predetermined physical activity (e.g., a rest period) than the period in which the user is participating in a predetermined physical activity (a lower heart rate for the same core temperature or work rate); the user having a relatively lower heart rate during the user having a first core body temperature than a heart rate that the user previously had while the user had the first core body temperature; the user having a relatively lower heart rate during the user participating in a first work rate than a heart rate that the user previously had while participating in the first work rate; a relatively quicker onset of sweating by the user than a previous onset of sweating by the user, e.g., sweating starts at a lower core body temperature; the user having a relatively higher sweat rate during the user having the first core body temperature than a sweat rate that the user previously had while the user had the first core body temperature; and the user having a relatively higher sweat rate during the user participating in the first work rate than a sweat rate that the user previously had while participating in the first work rate (e.g., a higher sweat rate for the same core temperature or work rate); etc.

In response to a determination that the user has successfully heat acclimatized to the predetermined environment, e.g., as illustrated by the “Yes” logical path of decision 802, a notification may be generated and/or output to the user that indicates that the user is able to fully participate in a physical activity in the predetermined environment, e.g., see operation 803. A personalized schedule of the user may be updated and or generated to reflect that the user is able to fully particulate in the physical activity in the predetermined environment. The personalized schedule may be similar to various personalized schedules described elsewhere herein, e.g., see operation 716 of method 700. Accordingly, in some approaches the personalized schedule may be based on, e.g., baseline health data of the user, activity-based health data of the user, environmental-based data of the user, etc. With continued reference to method 800, operation 806 includes outputting the personalized schedule for display on the user device of the user.

In some approaches, the determination that the user has successfully heat acclimatized to the predetermined environment may be based on a determination that a predetermined number of physiological changes, e.g., such as one or more of the physiological variables described elsewhere herein, have consistently occurred in the user, e.g., occurred a predetermined number of times over a predetermined number of intervals. This notification and/or updating of the user's personalized schedule may allow the user to begin a full workday in the heat, which may include wearing a full set of clothing and/or PPE required for the job. Such a notification and/or updating reduces the risk of heat injury and/or illness that may otherwise occur if the user were to fully participate in the physical activity before the user is fully heat acclimatized. Such a notification and/or updating may additionally and/or alternatively allow for managers to be sure that workers that they manage have been properly heat acclimatized before fully participating in the physical activity.

In contrast, in response to a determination that a predetermined number of physiological changes have not consistently occurred in the user, e.g., see “No” logical path of decision 802, it may be determined that the user has not successfully heat acclimatized to the predetermined environment. In some approaches, in response to a determination that the user has not successfully heat acclimatized to the predetermined environment, an alert may be output to a user device of the user and/or a user device of a second user, such as a manager of the user or other monitoring parties such as on-site medical team members. In some approaches, the alert may indicate that the user is not acclimatized to the heat, and therefore should not be assigned to a work schedule. A personalized schedule may additionally and/or alternatively be generated for the user to follow while participating in the physical activity, e.g., see operation 804. This personalized schedule preferably includes a less than full workload of the user, e.g., to allow the user to gradually gain heat acclimatization, and may be output for display on a user device of the user. The personalized schedule for heat acclimatizing the user may be generated using techniques similar to those described elsewhere herein, e.g., see method 700.

It should be noted that operation 803, operation 804 and/or operation 806 may be “optional” operations because in some approaches, method 800 may include merely detecting for a loss in heat acclimatization, e.g., de-acclimatization, subsequent to a determination that the user has successfully heat acclimatized to the predetermined environment. It may also be noted that the alerts and/or the personalized schedules may be output to the user device of the user using a variety of different mediums, e.g., SMS, dashboard, app, email, etc.

As described elsewhere above, the user may lose heat acclimatization subsequent to it being determined that the user has successfully heat acclimatized to the predetermined environment. In the event that the user loses heat acclimatization, but does not realize it and/or continues to fully participate in the physical activity, the user may experience heat related injury and/or illness. Accordingly, to preserve the health of the user, it may be determined whether the user has experienced a loss of heat acclimatization, e.g., has heat de-acclimatized, to the predetermined environment, e.g., see decision 808. The determination that the user has heat de-acclimatized to the predetermined environment, e.g., such as in an initial instance of the user de-acclimatizing to the predetermined environment, may be based on a detection of at predetermined trigger event. In one preferred approach, the trigger event may include one or more physiological changes. For example, the physiological changes may include changes in physiological data of the user such as a reversal of physiological changes that were previously observed during a heat acclimatization process of the user. In another preferred approach, the trigger event may include one or more predetermined weather events, e.g., such as an identified long-term change in weather. The predetermined weather events may be based on a weather forecast, an API call to a weather service, etc. In another preferred approach, the trigger event may include a detected absence of a worker, e.g., due to illness, vacation, etc. For example, the trigger event may be based on a determination that the user has not been located at a predetermined geographical location for at least a predetermined amount of time.

In response to a determination that the user has not heat de-acclimatized to the predetermined environment (e.g., as illustrated by the “No” logical path of decision 808), monitoring for the user becoming heat de-acclimatized to the predetermined environment may continue. For example, in some approaches, decision 808 may be ongoingly performed according to a predetermined interval for a predetermined number of times.

In contrast, in response to a determination that the user has heat de-acclimatized to the predetermined environment (e.g., as illustrated by the “Yes” logical path of decision 808), in some approaches, method 800 preferably includes performing a predetermined process for determining whether the user has heat re-acclimatized to the predetermined environment, e.g., see decision 814. In some approaches, method 800 may additionally include performing one or more operations to heat re-acclimatize the user to the predetermined environment, e.g., see operations 810-812. The predetermined process for determining whether the user has heat re-acclimatized to the predetermined environment may include one or more predetermined operations. For example, in one approach, an operation of the predetermined process including outputting a notification to a user device of the user and/or a user device of a second user, e.g., a manager of the first user, a coach of the first user, a family member, a medical professional of the user, etc. The notification may include an indication that the user has lost their heat acclimatization and should as a result participate in a predetermined re-acclimatization program. A personalized schedule for the user to follow in order to re-acclimatize the user to the predetermined environment may be generated, e.g., see operation 810. The personalized schedule is preferably individualized to the user as it may be based on baseline health data of the user, activity-based health data of the user and/or environmental-based data of the user. In some approaches, based on a determination that the user has previously successfully undergone a heat acclimatization process, e.g., see “Yes” of decision 802, it may be predicted that the user is likely to re-acclimatize relatively more quickly than the amount of time that it previously took for the user to initially heat acclimatize to the predetermined environment. For example, in some approaches, it may be predicted that the user may heat re-acclimatize in about two to three days.

Note that an alert that indicates that the user has heat de-acclimatized to the predetermined environment may additionally and/or alternatively be output to the user device of the user and/or a user device of a second user in response to the determination that the user has heat de-acclimatized to the predetermined environment, e.g., see operation 809.

The generated personalized schedule for the user to follow in order to re-acclimatize to the to the predetermined environment may in some approaches include suggestions, e.g., steps that the user may take to heat re-acclimatize to the predetermined environment. For example, a non-limiting list of suggestions includes, e.g., clothing modifications for the user, a geographical location for the user to reside at for a predetermined period of time, an amount and type of hydrating beverage for the user to consume, etc. The suggestions of the generated personalized schedule may in one approach additionally and/or alternatively include at least one instruction of when to begin participating in a physical activity and at least one instruction of when to stop participating in the physical activity. In such an approach, techniques similar to those described elsewhere herein for generating instruction of when to begin participating in a physical activity and at least one instruction of when to stop participating in the physical activity may be used to generate the suggestions, e.g., see method 300. In yet another approach, suggestions of the generated personalized schedule may be based on a predetermined ratio of techniques that were previously suggested to the user during an initial heat acclimatization process. For example, according to a more specific approach, where a personalized schedule output to a user device of the user during an initial heat acclimatization process instructed a user to only participate in physical activity for one hour a day, the generated personalized schedule for the user to follow in order to re-acclimatize to the to the predetermined environment may include a suggestion for the user to only participate in physical activity for one half of an hour a day in response to the predetermined ratio being ¹/₂. The predetermined ratio may be set and/or adjusted by, e.g., the user, the second user, etc. Accordingly, method 800 may include an algorithmically controlled process for optimizing the re-acclimatization of workers after loss of heat acclimatization due to short breaks away from the work site or short term changes in weather or working conditions.

Operation 812 of method 800 includes outputting the personalized schedule for display on a user device of the user, e.g., output via SMS, dashboard, app, email, known outputting techniques, etc.

It is determined whether the user has successfully heat re-acclimatized to the predetermined environment, e.g., see decision 814. The determination of whether the user has successfully heat re-acclimatized to the predetermined environment may in some approaches include determining whether the core body temperature of the user has increased a second predetermined amount from the baseline core body temperature of the user for a second predetermined amount of time during each of a predetermined number of second time intervals. According to some more specific approaches, each of the second time intervals may be about twenty-four hours and the predetermined number of second time intervals may be about one to three.

In response to a determination that the user has successfully heat re-acclimatized to the predetermined environment (e.g., as illustrated by the “Yes” logical path of decision 814), the personalized schedule may be updated and output to the user device of the user, e.g., see operation 816. The personalized schedule may in some approaches be updated to indicate that the user may fully participate in the physical activity based on the determination that the user has successfully heat re-acclimatized to the predetermined environment. For example, assuming that the physical activity is working, the personalized schedule may in some approaches be updated to include a full work shift in response to the determination that the user has successfully heat re-acclimatized to the predetermined environment. According to another example, assuming that the physical activity is participating in a sporting activity, the personalized schedule may in some approaches be updated to include a exercise routine in response to the determination that the user has successfully heat re-acclimatized to the predetermined environment. Operation 818 includes outputting the updated personalized schedule to the user device of the user.

An alert that indicates that the user has successfully heat re-acclimatized to the predetermined environment may additionally and/or alternatively be output to the user device of the user and/or a user device of a second user, e.g., see operation 826.

In contrast, in some approaches, in response to a determination that the user has not successfully heat re-acclimatized to the predetermined environment (e.g., as illustrated by the “No” logical path of decision 814), an alert may be output to the user device of the user and/or to a user device of a second user, e.g., see operation 820. The alert may be output to the user device of the second user to notify the second user that the user is still not heat re-acclimatized to the predetermined environment. This may enable the second user to take precautionary measures in order to prevent the user from becoming ill and/or injured as a result of the environmental conditions of the predetermined environment. The alert may additionally and/or alternatively be output to the user device of the user.

In response to the determination that the user has not successfully heat re-acclimatized to the predetermined environment, method 800 may optionally additionally and/or alternatively include modifying the personalized schedule of the user, e.g., see operation 822. For example, the personalized schedule of the user may be modified to incorporate alternative or additional techniques for the user to follow in order to re-acclimatize to the to the predetermined environment. Operation 824 of method 800 includes outputting the updated personalized schedule to the user device of the user.

it is important to note that user individualized heat re-acclimatization has heretofore not been considered and/or incorporated into conventional heat acclimatization efforts. As mentioned elsewhere above, this is because conventional techniques for heat acclimatization only consider a limited number of variables among workers, and ultimately do not account for the large physiological variability among individuals, e.g., biological sex, age, fitness level, acclimatization status, whether the worker is taking medication, what type and/or dosage of medication a worker is taking, etc. Moreover, conventional techniques for heat acclimatization are often designed from laboratory tests that utilize subject populations predominantly comprised of young, healthy men. Accordingly, typical heat acclimatization techniques any thereby related re-acclimatization techniques are not accurate for women and older individuals, e.g., such as women and individuals more than thirty-five years old. This does not ensure all workers equally heat acclimatize and/or re-acclimatize and as a result, users continue to be susceptible to heat-related injuries. In sharp contrast to the acclimatization deficiencies described above, various embodiments and approaches described herein ensure that a user is properly heat re-acclimatized subsequent to losing heat acclimatization and before resuming participation in a physical activity by determining whether determining whether the core body temperature of a user has increased a predetermined amount from a baseline core body temperature of the user for a predetermined amount of time during each of a predetermined number of time intervals. Overall, the heat re-acclimatization techniques of various approaches described herein are optionally specific to each individual user and thereby ensure that each user is receiving sufficient stimulus to re-acclimatize to heat, while simultaneously preventing heat-related injuries and illnesses that may otherwise occur subsequent to heat acclimatization, and as a result of, e.g., predetermined weather events, the user not being located at a predetermined geographical location for at least a predetermined amount of time, changes in physiological data of the user, etc. Accordingly, the inventive discoveries disclosed herein with regards to individualized heat re-acclimatization techniques proceed contrary to conventional wisdom.

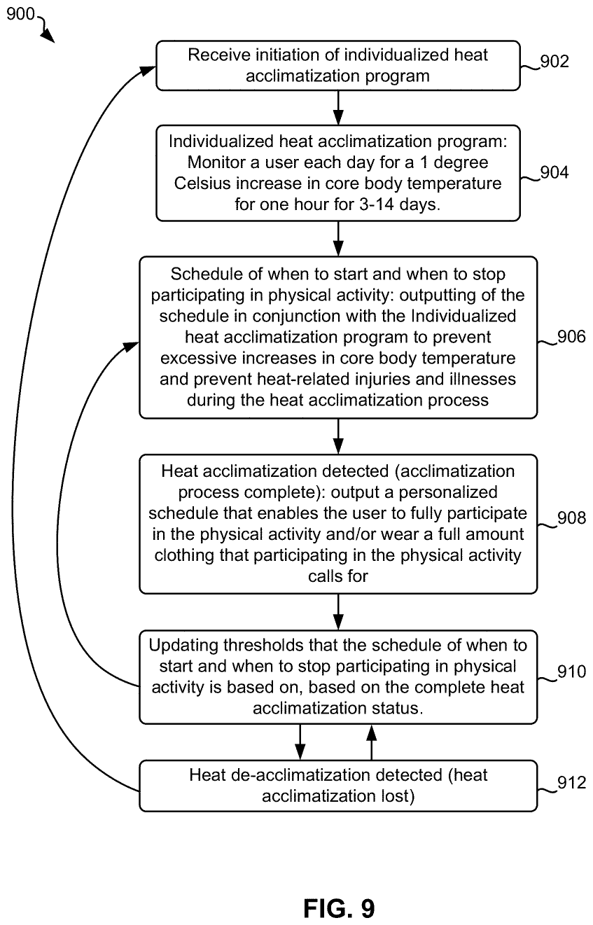

FIG. 9 shows a method 900, in accordance with one embodiment. As an option, the present method 900 may be implemented in devices such as those shown in the other FIGS. described herein. Of course, however, this method 900 and others presented herein may be used to provide applications which may or may not be related to the illustrative embodiments listed herein. Further, the methods presented herein may be carried out in any desired environment. Moreover, more or less operations than those shown in FIG. 9 may be included in method 900, according to various embodiments. It should also be noted that any of the aforementioned features may be used in any of the embodiments described in accordance with the various methods. For example, as will be described in further detail below, method 900 may be incorporated into one or more other methods herein, e.g., such as method 300 and/or method 700. Accordingly, FIG. 9 may represent an overarching flow between the personalized schedules for displaying to a user participating in activity to thereby mitigate productivity losses and user injuries and/or illnesses, e.g., see method 300, the individualized heat acclimatization program, e.g., see method 700, and the individualized heat re-acclimatization subsequent to a determination that a user has become heat de-acclimatized, e.g., see method 800.

Operation 902 includes receiving an initiation of an individualized heat acclimatization program.

Operation 904 is related to the individualized heat acclimatization program. For example, operation 904 includes monitoring a user each day for a 1 degree Celsius increase in core body temperature for one hour for 3-14 days.

Operation 906 is related to the schedule of when to start and when to stop participating in physical activity. For example, operation 906 includes outputting of the schedule in conjunction with the individualized heat acclimatization program to prevent excessive increases in core body temperature and prevent heat-related injuries and illnesses during the heat acclimatization process.

In operation 908, heat acclimatization is detected (acclimatization process complete). More specifically, operation 908 includes outputting a personalized schedule that instructs the user to fully participate in the physical activity and/or wear a full amount clothing that participating in the physical activity calls for.

In operation 910, thresholds that a schedule of when to start and when to stop participating in physical activity is based on are updated. The updating may be performed based on the complete heat acclimatization status.

In operation 912, heat de-acclimatization is detected, e.g., heat acclimatization is lost. Based on detecting the heat de-acclimatization, operation 902 may in some approaches be performed, e.g., see logical path of operation 912 return to operation 902.

FIGS. 10A-10B depict plots 1000, 1020 of data of a user pre-acclimatized and post-acclimatized, in accordance with various embodiments. As an option, the present plots 1000, 1020 may be implemented in conjunction with features from any other embodiment listed herein, such as those described with reference to the other FIGS. Of course, however, such plots 1000, 1020 and others presented herein may be used in various applications and/or in permutations which may or may not be specifically described in the illustrative embodiments listed herein. Further, the plots 1000, 1020 presented herein may be used in any desired environment.

Referring first to FIG. 10A, plot 1000 contrasts a core temperature and/or a work rate of a user with the user's heart rate in beats per minute (bpm). More specifically, the plot 1000 includes a first line 1002 that represents the user pre-acclimatization and a second line 1004 that represents the user post-acclimatization. Several physiological changes that may occur during the heat acclimatization process of the user are shown in the plot 1000. For example, at a first plot point 1006, data associated with the user having a relatively lower heart rate at rest is illustrated. In another example, at a second plot point 1008, data associated with the user having a relatively lower heart rate for the same core temperature or work rate is illustrated.

Referring now to FIG. 10B, plot 1020 contrasts a core temperature and/or a work rate of a user with the user's sweat rate in milliliters per hour (ml/h). More specifically, the plot 1020 includes a first line 1024 that represents the user pre-acclimatization and a second line 1022 that represents the user post-acclimatization. Several physiological changes that may occur during the heat acclimatization process of the user are shown in the plot 1020. For example, at a first plot point 1026, data associated with the user having a relatively earlier onset for sweating, e.g., sweating starts at lower core body temperature, is illustrated. More specifically, in the post-heat acclimatization associated second line 1022, the user is shown to begin sweating relatively sooner, and have a relatively higher max sweat rate, e.g., see plateaus 1030, than similar metrics of the pre-acclimatization associated first line 1024. In another example, at a second plot point 1028, data associated with the user having a relatively higher sweat rate for the same core temperature or work rate is illustrated.

Individualized Sweat Rate and Hydration Alert System

Workers in many industries across the world are exposed to dangerously hot working conditions. As mentioned elsewhere above, heat-related injuries and illnesses cost billions of dollars around the world each year in medical care and productivity losses. For example, 30% of individuals who work in relatively elevated temperature environments report productivity losses. A majority of heat-related injuries are mitigated with proper rest and recovery, however, exertional heat stroke and death can occur in some instances as a result of a worker's core body temperature reaching certain elevated levels and/or as a result of dehydration of a worker. For context, for each one degree Fahrenheit increase in summer temperatures, the likelihood of heat-related deaths increases up to 37%. Many of these heat-related problems result from workers over-exerting themselves because either a) workers themselves do not know when they need to stop working and take a break and/or b) the managers of workers do not know when to instruct workers to stop working and take a break, etc. Most often these heat injuries and illnesses occur while working in a relatively hot work environment and/or wearing heavy protective gear (e.g. PPE) that prohibits heat loss. In addition, PPE clothing designed to keep industrial workers safe can increase the danger of heat-related illnesses in hot, humid, and even cool work environments.

Many heat-related injuries and illnesses occur despite managers utilizing work shift schedules and/or encouraging workers to adequately hydrate. This is because these conventional measures only consider a limited number of variables among workers, and ultimately do not account for the large physiological variability among individuals, e.g., biological sex, age, fitness level, acclimatization status, whether the worker is taking medication, what type and/or dosage of medication a worker is taking, etc. Moreover, conventional work schedules are often designed from laboratory tests that utilize subject populations predominantly comprised of young, healthy men. Accordingly, typical conventional schedules are not accurate for women and older individuals, e.g., such as women and individuals more than thirty-five years old.

As the climate changes worldwide, workers who work outside are at higher risk for heat-related injuries. Research has identified 285 construction worker deaths directly related to heat from 1992 to 2016. 78% of those deaths occurred during the hot summer months between June and August. As global warming and/or climate change is leading to relatively hotter temperatures across the globe, the risk of heat-related deaths is also increasing. As noted elsewhere above, for each one degree Fahrenheit increase in summer temperatures, the likelihood of heat-related deaths increase up to 37%.

Proper hydration and fluid intake during outdoor activities is a critical part of preventing heat-related injuries. As the core body temperature of a user rises, the heart rate of the user also increases to pump more blood to the skin surface to dissipate the excess body heat in the form of sweat. Individuals who lose a percentage of body mass through sweat can exhibit an increased heart rate and core body temperature and a decrease in cognitive awareness. Dehydration also causes users to be more susceptible to heat-related injuries and illnesses. Blood is made up of about fifty percent water, and therefore, maintaining hydration and replacing water loss due to sweat is an important step to preventing injury and/or illness of a user. Conventional techniques to ensure that a user maintains proper hydration typically include relying on managers of the user to remind the user to hydrate and/or relying on the user themself to remember and attempt to properly hydrate. Unfortunately, many heat-related injuries like dehydration result from users not realizing that they are becoming dehydrated and require the consumption of fluids. Moreover, current fluid drinking guidelines do not adequately hydrate users, as they rely on a ‘one size fits all’ approach where users genetic makeups substantially vary.

In sharp contrast to the deficiencies described above with regards to user hydration, various approaches described herein enable productivity of one or more users by generating a personalized schedule for a user to consume fluids based on health data of the user. Following the schedule that is specifically individualized to the user based on the user's health data, injury and/or illness that would otherwise experience as a result of becoming dehydrated, are avoided.

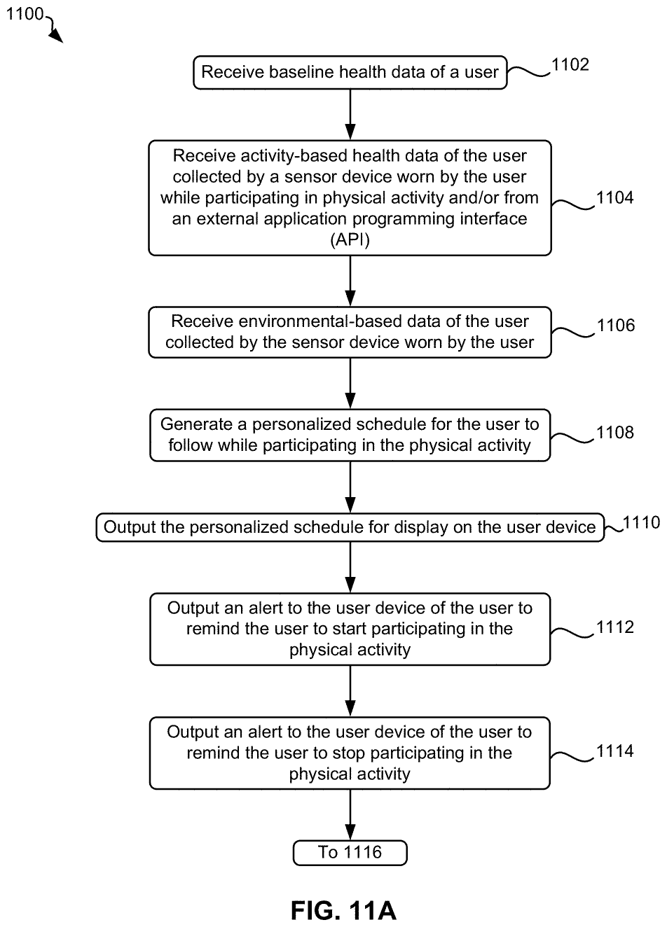

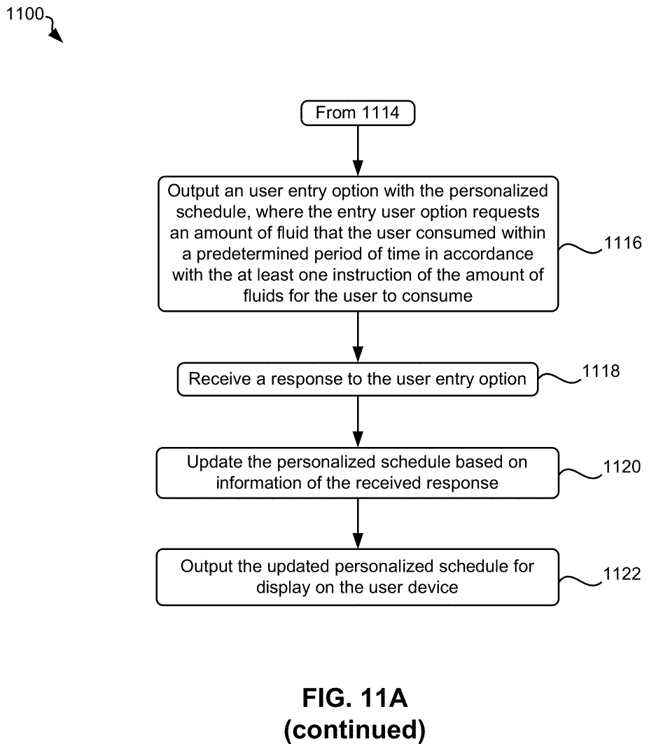

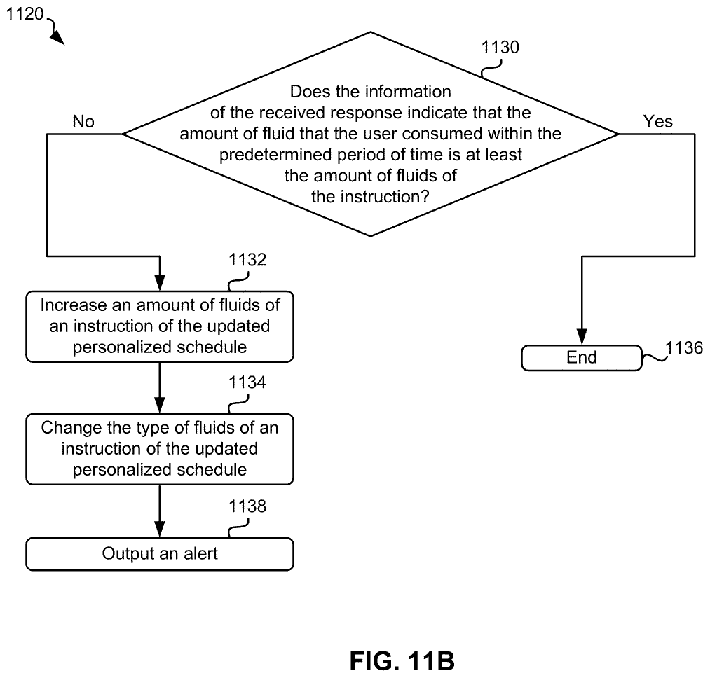

FIG. 11A shows a method 1100, in accordance with one embodiment. As an option, the present method 1100 may be implemented in devices such as those shown in the other FIGS. described herein. Of course, however, this method 1100 and others presented herein may be used to provide applications which may or may not be related to the illustrative embodiments listed herein. Further, the methods presented herein may be carried out in any desired environment. Moreover, more or less operations than those shown in FIG. 11A may be included in method 1100, according to various embodiments. It should also be noted that any of the aforementioned features may be used in any of the embodiments described in accordance with the various methods.

Operation 1102 includes receiving baseline health data of a user. Note that the received baseline health data may be pre-acquired, e.g., using a known type of health data input module, using a known technique for gathering health data from one or more sources, based on one or more questionnaires, processing images of a user using one or more known types of recognition techniques, etc. A non-limiting list of the baseline health data of the user may include, e.g., medical history data, anthropometric data such as weight and/or height, age, workday length and/or shift length, biological sex, a determined body mass index, etc.

Activity-based health data of the user, e.g., biometric data, collected by a sensor device worn by the user while participating in physical activity is received, e.g., see operation 1104 of method 1100. In some approaches, the activity-based health data of the user may additionally and/or alternatively be received from an external application programming interface (API) to a weather service or station. Accordingly, in some approaches, the sensor device may be configured to continually, e.g., according to a predetermined interval, and non-invasively, collect predetermined metrics of the user using one or more known types of sensors, e.g., a camera, a heartbeat sensor, a temperature sensor for determining a temperature of the user, a humidity sensor, a motion sensor, a global positioning system, a proximity sensor, a gyroscope, an accelerometer, a microphone, a heat flux sensor, etc.

The activity-based health data may depend on the approach. For example, in some approaches, the activity-based health data may include physiological data of the user, e.g., ambient temperature, ambient humidity, skin temperature of the user, skin humidity of the user measured by the sensor device, near skin humidity of the sensor, a perspiration rate of the user, a heart rate of the user such as measured via photoplethysmography or via one or more known techniques, an activity type that the user is determined to be engaged in and data associated therewith, a rate of movement of the user, etc. In another approach, the activity-based health data may additionally and/or alternatively include motion data.

It should be noted that the type of physical activity that the activity-based health data is based on may depend on the approach. For example, in some approaches, the activity-based health data may be collected while the user is participating in non-stationary physical activity, e.g., running, walking, swimming, performing a work task, etc. In contrast, the activity-based health data of the user may additionally and/or alternatively be collected by the sensor device worn by the user while participating in stationary activity, e.g., sleeping, taking a break at work, sitting stationary, awake in the user's home, driving to work, watching a user device, etc.

Operation 1106 includes receiving environmental-based data of the user collected by the sensor device worn by the user. Data based on clothing worn by the user and/or clothing expected to be worn by the user may additionally and/or alternatively be received in an optional operation of method 1100. The environmental-based data and/or the clothing-based data may be similar to and/or collected using similar techniques to the environmental-based data and/or the clothing-based data described elsewhere herein, e.g., see operation 305 of method 300 and operation 706 of method 700.

A personalized schedule for the user to follow while participating in the physical activity may be generated based on the baseline health data of the user, the activity-based health data of the user and the environmental-based data of the user. In some preferred approaches, generating the personalized schedule may include determining an amount and or type of fluids for the user to consume while participating in the physical activity to maintain hydration of the user and prevent dehydration of the user. Such a determination my be performed using one or more of the techniques described below.

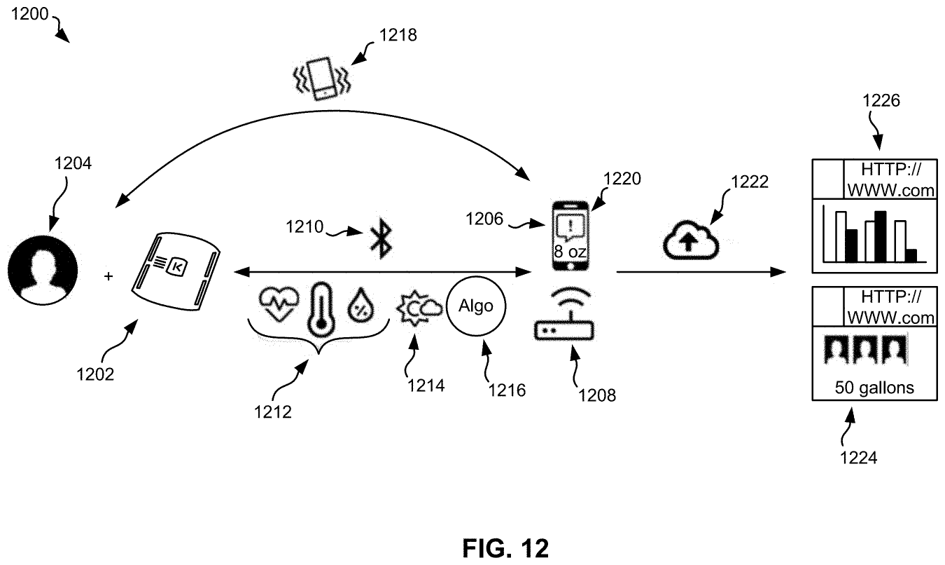

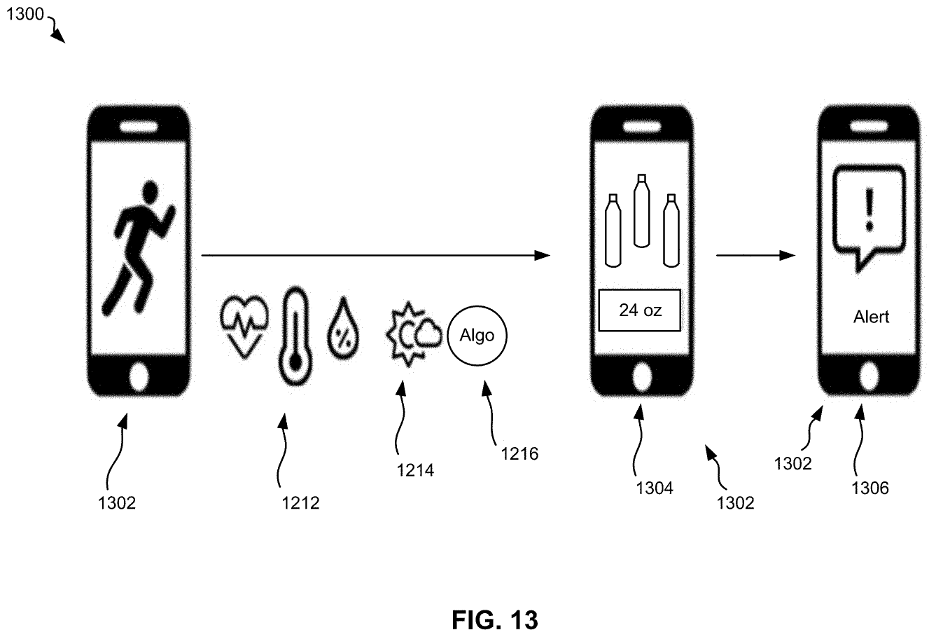

In one approach, the amount and/or type of fluids for the user to consume while participating in the physical activity may be determined using a table that includes a plurality amounts and/or types of fluids that are pre-associated to values of the received data. For example, the amount and or type of fluids for the user to consume while participating in the physical activity may be determined by accessing the table and determining which amount and/or type of fluid correspond, e.g., are pre-correlated, to values of the received data, e.g., baseline health data of the user, activity-based on health data of the user, environmental-based data of the user, data based on clothing worn by the user and/or clothing expected to be worn by the user, etc. In some approaches, the received data may be used to calculate a second metric that is pre-correlated with an amount and/or type of fluids. For example, in one approach, the received data may be used to calculate a sweat rate of the user. In some approaches, the sweat rate of the user may be calculated using a standard lookup tables that is based on the received data. In some other approaches, the sweat rate may be calculated using, e.g., demographic data, machine learning models such as linear regression, black box models using the physiological data (from the device sensors), user feedback, based on environmental conditions throughout a workday of the user, etc. The sweat rate may additionally and/or alternatively be based on basic demographic information, through a guided activity, e.g., see operation 1116 and operation 1118 of method 1100 and FIGS. 12-13, and/or automatically through daily work activity. In one or more of such approaches, the amount and/or type of fluids for the user to consume while participating in the physical activity may be pre-correlated with the calculated sweat rate of the user.

The amount and/or type of fluids for the user to consume while participating in the physical activity may be determined using data modeling. In one approach a database may be generated by the data modeling using ongoingly collected and/or updated values of the received data. Data of the database may be comparatively analyzed with the received data of a user in order to determine the amount and/or type of fluids for the user to consume while participating in the physical activity. More specifically, in some approaches, the comparative analysis may include comparing data of the user to data of the database to identify amounts and/or types of fluids for the user to consume while participating in the physical activity that were previously incorporated into a personalized schedule of a user having data within a predetermined degree of similarity, e.g., which may be the user or another user, and did not result in the user becoming dehydrated while following the personalized schedule. In such approaches, the more data that is incorporated into the modeling and database, the more likely a generated personalized schedule is to prevent the user from becoming dehydrated. In some other approaches, one or more known techniques of data modeling may additionally and/or alternatively be incorporated into the generation of the personalized schedule for the user to follow.

In some approaches, the amount and/or type of fluids for the user to consume while participating in the physical activity may additionally and/or alternatively be determined based on measured and/or predicted environmental values, e.g., such as the receives environmental-based data of the user collected by the sensor device worn by the user. One or more of such approaches may utilize API calls to a weather source and/or network connected environmental sensors. In one approach, such data may be incorporated into a data modeling technique described above in order to determine the amount and/or type of fluids for the user to consume while participating in the physical activity.

Data collection and research based on trial and analysis of results may additionally and/or alternatively be used for determining the amount and/or type of fluids for the user to consume while participating in the physical activity. Note that in one or more of such approaches, the trial process may be performed in a lab or any other controlled setting to ensure that users are not injured as a result of following a personalized schedule that is based on a minimal amount of trial and analysis. The trial and analysis of results may in one approach include increasing the amount of fluid and/or increasing the electrolyte content of the type of fluid that the user is to consume in response to a determination that the user following the personalized schedule becomes dehydrated or is sweating at a rate that is predicted to result in the user becoming dehydrated. In contrast, the trial and analysis of results may additionally and/or alternatively include decreasing the amount of fluid and/or decreasing the electrolyte content of the type of fluid that the user is to consume in response to a determination that the user following the personalized schedule does not become dehydrated or is not sweating at a rate that is predicted to result in the user becoming dehydrated. Note that in some approaches, the electrolyte content of the type of fluid that the user is to consume may consider and account for sodium that the user consumes in food. This measure of sodium may be determined using known techniques, e.g., such as user entry in a user option output with the personalized schedule.

Test case research and extrapolation based on results may additionally and/or alternatively be used for determining the amount and/or type of fluids for the user to consume while participating in the physical activity. For example, in some approaches, the received data of the user may be applied to one or more known techniques of test case research and extrapolation to generate the personalized schedule for the user to follow.

One or more known types of calculations may be additionally and/or alternatively be used for determining the amount and/or type of fluids for the user to consume while participating in the physical activity. For example, in some approaches, the received data of the user may be applied to a black box equation having an output that includes an amount and/or type of fluids for the user to consume while participating in the physical activity. In another approach, the received data of the user may be applied to a known type of machine learning algorithm to generate and/or ongoingly update the personalized schedule for the user to follow.

As will be described below, generating the personalized schedule may in some approaches additionally and/or alternatively include assigning a predetermined amount of the determined amount of liquid to one or more portions of the personalized schedule. Depending on the approach, the personalized schedule may instruct the user to consume the entire amount of liquid at a single predetermined time. In contrast, in some other approaches, instructions of the personalized schedule may specify more than one predetermined amount of time to consume about a predetermined amount of the liquid, e.g., a gulp, a sip, a specified liquid volume, a predetermined number of seconds of drinking the liquid, etc. In one or more of such approaches in which the personalized schedule specifies more than one predetermined amount of time to consume about a predetermined amount of the liquid, the amount of liquid may be divided according to a predetermined ratio. For example, the determined amount of liquid may be equally dividing over a predetermined number of breaks, e.g., such as established by the at least one instruction of when to start participating in the physical activity and the at least one instruction of when to stop participating in the physical activity described elsewhere herein. In another non-limiting example, the determined amount of liquid may be unequally divided over a predetermined number of breaks, e.g., dividing the amount of liquid over a predetermined number of breaks according to a predetermined pattern where a relatively hotter part of the day is at least initially assigned a relatively greater amount of the liquid than an amount of the liquid at least initially assigned to a relatively cooler part of the day, etc.

As mentioned elsewhere above, the personalized schedule of method 1100 may in some approaches include at least one instruction of when to start participating in the physical activity and at least one instruction of when to stop participating in the physical activity in order to maintain hydration of the user and prevent dehydration of the user. The instructions of when to start participating in the physical activity and when to stop participating in the physical activity in order to maintain hydration of the user and prevent dehydration of the user may be generated using similar techniques to techniques described elsewhere herein for generating instructions of when to start participating in a physical activity and instructions of when to stop participating in a physical activity, e.g., see method 300, method 800, etc. For example, the received data may be used to determine initial start and stop instructions that are predicted to maintain and/or improve the user's hydration levels. Start and stop instructions thereafter may be adjusted based on how the user's hydration levels are impacted by the initial start and stop instructions and/or based on the user's hydration levels are impacted by the at least one instruction of the amount and/or type of fluids for the user to consume while participating in the physical activity (provided that the user attest to consuming the liquids accordingly).

The personalized schedule is output for display on the user device, e.g., see operation 1110 of method 1100. Depending on the approach, the personalized schedule may be output using one or more techniques for outputting a personalized schedule described elsewhere herein, e.g., SMS, a tablet, a phone app, a desktop app, dashboard, etc.

Operation 1112 includes outputting an alert to the user device of the user to remind the user to start participating in the physical activity. Moreover, operation 1114 includes outputting an alert to the user device of the user to remind the user to stop participating in the physical activity. In some approaches one or more of such alerts may additionally and/or alternatively be output to any one or more other devices, e.g., a user device of a second user, a watch of the user, a phone of the user, the sensor device of the user, etc. The alert may be output with an indicator specification and/or pattern described elsewhere above, e.g., a haptic motor, a visual indicator, an audio alert, etc. The timing at which one or more of the reminder alerts are output may depend on the approach. For example, one or more of the alerts may be output, e.g., at the time that the user is to start participating in the physical activity, at a time prior to the time that the user is to start participating in the physical activity, output subsequent to a time that the user is to start participating in the physical activity in response to a determination that the user has not started to participate in the physical activity at a time indicated in the instruction to start participating in the physical activity, at the time that the user is to stop participating in the physical activity, at a time prior to the time that the user is to stop participating in the physical activity, output subsequent to a time that the user is to stop participating in the physical activity in response to a determination that the user has not stopped participating in the physical activity at a time indicated in the instruction to stop participating in the physical activity, etc.

Moreover, it should be noted depending on the approach, method 1100 may include outputting a reminder alert and predetermined number of times, for any number of instructions that the personalized schedule may include, e.g., once for each of the instructions, three times for only a first of the instructions, once for every other instruction, etc. In some approaches, the reminder alert output settings may be set and/or adjusted by, e.g., the user, a second user, a manufacturer of the sensor device, etc.

In some approaches a user entry option may be output with the personalized schedule, e.g., see operation 1116. The entry user option may preferably request an amount of fluid that the user consumed within a predetermined period of time in accordance with the at least one instruction of the amount of fluids for the user to consume. For context, the amount of fluid that the user has consumed within the predetermined period of time in accordance with the at least one instruction of the amount of fluids for the user to consume may be used to determine whether instructions potentially sent to the user thereafter should include instructions with an increased amount of fluid, e.g., as a result of the user failing to consume, within the predetermined period of time, the amount of fluid included in an instruction of a previous instruction, or alternatively, whether instructions potentially sent to the user thereafter should include instructions with a decreased amount of fluid, e.g., as a result of the user consuming, within the predetermined period of time, at least the amount of fluid included in an instruction of a previous instruction. The predetermined period of time may vary depending on the approach, e.g., one hour, one day, the period of time between at least one instruction of when to start participating in the physical activity and at least one instruction of when to stop participating in the physical activity, a period of time that includes the predicted hottest part of a day, etc.

In some other approaches, a user entry option output with the personalized schedule may additionally and/or alternatively include a plurality of fluid types for the user to select. One or more received user selections of types of fluids may be incorporated into the personalized schedule thereafter. It should be noted that the amount of fluid of a personalized schedule may vary depending on the type of fluid. For example, in some approaches, a personalized schedule may include a relatively lesser amount of a fluid that includes at least a predetermined electrolyte concentration than an amount of a fluid that does not include at least the predetermined electrolyte concentration.

A response to the user entry option is received in method 1100, e.g., see operation 1118. The response may include, e.g., selection(s) of one or more user selectable options of the user entry option, a specified amount of fluid consumed by the user within a predetermined amount of time, a specified amount of fluid not consumed by the user within a predetermined amount of time, etc.

The personalized schedule may be updated based on information of the received response, e.g., see operation 1120. Looking to FIG. 11B, exemplary sub-processes of updating the personalized schedule based on information of the received response are illustrated in accordance with one embodiment, one or more of which may be used to perform operation 1120 of FIG. 11A. More specifically, the exemplary sub-processes of FIG. 11B is included to illustrate the series of determinations that may be performed across the intervals. However, it should be noted that the sub-processes of FIG. 11B are illustrated in accordance with one embodiment which is in no way intended to limit the invention.

With reference now to FIG. 11B, in some approaches, updating the personalized schedule based on information of the received response may include determining whether the information of the received response indicates that the amount of fluid that the user consumed within the predetermined period of time is at least the amount of fluids of the instruction, e.g., see sub-operation 1130. Known techniques may be utilized to make such a determination in some approaches. Sub-operation 1130 may additionally and/or alternatively include determining whether the information of the received response indicates that the user started participating in the physical activity and stopped participating in the physical activity according to the personalized schedule.

In response to a determination that the information of the received response indicates that the amount of fluid that the user consumed within the predetermined period of time is at least the amount of fluids of the instruction, e.g., as illustrated by the “Yes” logical path of sub-operation 1130, and/or in response to a determination that the information of the received response indicates that the user started participating in the physical activity and stopped participating in the physical activity according to the personalized schedule, method 1100 may end, e.g., see sub-operation 1136. In contrast, in response to a determination that the information of the received response indicates that the amount of fluid that the user consumed within the predetermined period of time is not at least the amount of fluids of the instruction, e.g., as illustrated by the “No” logical path of sub-operation 1130, and/or in response to a determination that the information of the received response indicates that the user did not start participating in the physical activity and did not stop participating in the physical activity according to the personalized schedule, an amount of fluids of an instruction of the updated personalized schedule may be increased, e.g., see sub-operation 1132. The amount of fluids of an instruction of the updated personalized schedule may be increased a predetermined amount, e.g., a predetermined number of milliliters, a predetermined amount in addition to any unconsumed portion of the amount of fluids of the previous instruction, etc. In some approaches, the type of fluid of the instruction of the updated personalized schedule may additionally and/or alternatively be different than a type of fluid of the instruction of the personalized schedule, e.g., see sub-operation 1134. For example, in one approach, the type of fluid of the instruction of the updated personalized schedule have a greater electrolyte concentration than an electrolyte concentration of the type of fluid of the instruction of the personalized schedule in response to a determination that the information of the received response indicates that the amount of fluid that the user consumed within the predetermined period of time is not at least the amount of fluids of the instruction, e.g., where the type of fluid of the instruction of the personalized schedule is water and the type of fluid of the instruction of the updated personalized schedule is an electrolyte replenishing liquid.

Moreover, in some approaches, in response to the determination that the information of the received response indicates that the amount of fluid that the user consumed within the predetermined period of time is not at least the amount of fluids of the instruction, e.g., as illustrated by the “No” logical path of sub-operation 1130, an alert may be output, e.g., see sub-operation 1138. The alert may be a “danger” alert and may specify that the user has failed to consume a predetermined amount of fluids and therefore may be dehydrated. The alert may be output to a user device of the user and/or a user device of another user that is in a position to assist or remind the user to take steps to re-hydrate.

It should be noted that adjustment of the amount of fluids and/or type of the fluids may additionally and/or alternatively be based on a one or more determinations of whether the user is becoming relatively more dehydrated over the predetermined period of time. Such determinations may be based on comparisons of ongoingly received data, e.g., activity-based health data of the user, environmental-based data of the user, etc.

With reference again to FIG. 11A, operation 1122 includes outputting the updated personalized schedule for display on the user device.

It is importan o note that user individualized hydration plans have heretofore not been considered and/or incorporated into conventional hydration efforts. As mentioned elsewhere above, this is because conventional techniques for user hydration only consider a limited number of variables among workers, and ultimately do not account for the large physiological variability among individuals, e.g., biological sex, age, fitness level, acclimatization status, whether the worker is taking medication, what type and/or dosage of medication a worker is taking, etc. Moreover, conventional techniques for user hydration are often designed from laboratory tests that utilize subject populations predominantly comprised of young, healthy men. Accordingly, typical heat acclimatization techniques any thereby related re-acclimatization techniques are not accurate for women and older individuals, e.g., such as women and individuals more than thirty-five years old. This does not ensure all workers adequately hydrate and as a result, users continue to be susceptible to heat-related injuries. In sharp contrast to the hydration deficiencies described above, various embodiments and approaches described herein ensure that a user is properly hydrated by generating a personalized schedule for the user to follow while participating in the physical activity based on the baseline health data of the user, the activity-based health data of the user and the environmental-based data of the user. Overall, the hydration techniques of various approaches described herein are optionally specific to each individual user and thereby ensure that each user is receiving sufficient hydration measures to participate in physical activity, while simultaneously preventing heat-related injuries and illnesses that may otherwise occur while being in relatively hot environmental conditions. Accordingly, the inventive discoveries disclosed herein with regards to individualized hydration techniques proceed contrary to conventional wisdom.

FIG. 12 depict system 1200, in accordance with one embodiment. As an option, the present system 1200 may be implemented in conjunction with features from any other embodiment listed herein, such as those described with reference to the other FIGS. Of course, however, such system 1200 and others presented herein may be used in various applications and/or in permutations which may or may not be specifically described in the illustrative embodiments listed herein. Further, the system 1200 presented herein may be used in any desired environment.

The system 1200 of FIG. 12 illustrates an implementation of a hydration alert system, e.g., which may be implemented using similar to the hydration techniques described in method 1100. In some approaches, the hydration alert system may include a wearable sensing unit, mobile phone and/or a Bluetooth Hub, a Cloud service, etc. The system 1200 may include a wearable sensor device 1202 which may be attached to a user's 1204 upper arm via a known type of adjustable elastic band/strap. In some approaches, the wearable sensor device 1202 may connect to a mobile device 1206 and/or a Bluetooth Hub 1208, e.g., via a Bluetooth connection 1210. As illustrated in metric 1212, the wearable sensor device 1202 is preferably configured to collect health data of the user, e.g., heart rate, temperature, motion, humidity, and/or local environmental conditions (such as via a weather API), e.g., see metric 1214. A predetermined algorithmic process 1216 may be used to apply the data of the wearable sensor device 1202 and/or the environmental conditions, along with baseline health data which may be collected during registration of the user, e.g., including age, height, weight, sex, workday length, medical history, etc., to determine a quantity of fluids for the user to consume and a number of breaks for the user to take during the workday to maintain hydration and avoid dehydration, e.g., see operation 1108 of method 1100. Real-time alert notifications may be output to the user via the mobile phone application or SMS on the mobile device 1206 and/or to the user arm via a series of strong haptic vibrations, e.g., see haptic signal 1218, and/or visual indication. The user may be prompted to enter the quantity of fluid consumed during the hydration alert break, e.g., see the outputting of user entry options with the personalized schedule in method 1100. In some approaches, the personalized schedule of the user may be updated on a daily basis based on a determination of the user's individual total body sweat rate, which may be based on the user's physiological and environmental data collected throughout the workday. Note that in some approaches, user health data and/or metrics of a user's personalized schedule may be viewable by any one or more predetermined users, e.g., the user, a manager of the user, a supervisor of the user, a scheduler of the user at a work setting, a coach of the user, a medical professional, etc. Some second users that manage the user may also receive such updates and alerts that the user receives.

The predetermined algorithmic process 1216 may in some approaches include and/or be created using one or more of the techniques described elsewhere herein, e.g., standard lookup tables using demographic data, machine learning models such as linear regression, black box models using the physiological data (from the device sensors), user feedback, and environmental conditions throughout the workday, other determination techniques described elsewhere herein (e.g., see operation 1108), etc. Moreover, the predetermined algorithmic process 1216 may be created from basic demographic information, through a guided activity (which may be selected by the user as described elsewhere herein), and/or automatically through daily work activity. Depending on the approach, logic of the predetermined algorithmic process 1216 may be stored locally on the device, on a mobile application, on a cloud service, etc.

System 1200 may additionally and/or alternatively include a mobile software application 1220 and the Bluetooth hub 1208 connected to a cloud service 1222 via a cellular and/or local network. If the user is a member of an organization's team, the hydration alerts may be recorded and available via a known type of team view dashboard webpage 1224 and/or an analytics web dashboard 1226. The team dashboard 1226 may display the user alerts and the calculated amount of fluids of instructions of the personalized schedule of each user that is a part of a team. In another approach, the analytics web dashboard 1226 may provide detailed views for multiple locations, including alerts, physiological, and environmental data. The dashboard webpage 1224 may in some approaches be a team website portal that illustrates user locations, and teams stop work alerts, team hydration details, etc. Moreover, in another approach, the web dashboard 1226 may be an analytics website portal that illustrates teams, locations, alerts, hydration plans, recommendations, etc.

FIG. 13 depict system 1300, in accordance with one embodiment. As an option, the present system 1300 may be implemented in conjunction with features from any other embodiment listed herein, such as those described with reference to the other FIGS. Of course, however, such system 1300 and others presented herein may be used in various applications and/or in permutations which may or may not be specifically described in the illustrative embodiments listed herein. Further, the system 1300 presented herein may be used in any desired environment.

System 1300 illustrates a guided activity hydration update. System 1300 includes a user device 1302 that may be in communication with a sensor device, e.g., via a Bluetooth connection. A personalized schedule 1304 for the user to follow while participating in physical activity may be generated based on baseline health data of the user, activity-based health data of the user (see metrics 1212) and the environmental-based data of the user (see metric 1214). The personalized schedule 1304 may be output for display on the user device 1302, and a mobile application may display an updated personalized schedule that is based on guided activity of the user. Moreover, updated alerts 1306 may be generated and displayed on the user device 1302, e.g., via notifications and device vibrations.

Individualized heat susceptibility alert system

As described elsewhere herein, there are a variety of factors that may lead to heat injury and/or illness of an individual. For example, some of these factors may be external factors, e.g., such as environmental conditions. In contrast, some of these factors may additionally and/or alternatively be internal factors and/or intrinsic factors of the individual, e.g., such as a disease that the user has. Certain diseases, medications, and/or past events may cause individuals to be relatively more susceptible to heat related injuries and illnesses than the individual would otherwise be without such factors. Additionally, an individual's workload and/or an individual's heat load may accumulate throughout a predetermined period of time, e.g., such as throughout a work week, to cause the individual to be more susceptible to heat-related injuries and/or illnesses each day. Moreover, certain genetic traits, some of which are still unknown, may cause heat intolerance in otherwise healthy individuals. In other words, such individuals may be unable to truly acclimatize to the heat and, therefore, may always be more susceptible to heat-related injuries and illnesses. Accordingly, for at least these individuals, it is important for the individual themselves as well as a manager of the individual, e.g., such as a manager where the individual is a worker, to understand whether the individual has an underlying condition and/or genetic predisposition that may cause the individual to be more susceptible to heat and/or be heat intolerant, and/or whether the individuals previous day's work, or cumulative workload throughout the week, makes the individual more susceptible to heat injuries and/or illnesses during the individual's next day at work.

Various approaches described herein include a two-part heat susceptibility notification (alert) system. In some of such approaches, the first part of the system may be configured to notify a user, such as a worker, and/or a manager of the user of any underlying conditions or genetic predisposition (at baseline) that may cause the user to be relatively more susceptible to heat-related injuries and/or illnesses. Moreover, a second part of the system may be configured to notify the user and/or a manager of the user, on a daily basis, about the user's heat susceptibility level, which may be based on the previous day's workloads. As will be described in greater detail elsewhere herein, depending on the approach, to execute such a notification system, various types of information may be collected and used, e.g., the user's medical history information, e.g., that the user fills out when creating an account, physiological data of a user that is collected while the user participates in an optional guided exercise activity to determine heat tolerance of the user, physiological data that is collected from the user on a daily basis, subjective worker feedback via daily surveys, weather data (via API), etc. For example, in some approaches, one or more types of such data may be integrated into machine learning models that will stratify workers' heat risk at baseline and each day as low, moderate, or high, so that workers and/or managers of one or more workers are enabled to prevent heat-related injuries and/or illnesses on a job site based on a modification of the workload of the worker via a work/rest schedule of a worker, based on a modification of shift times of a worker, based on a modification of job tasks of a worker, etc.

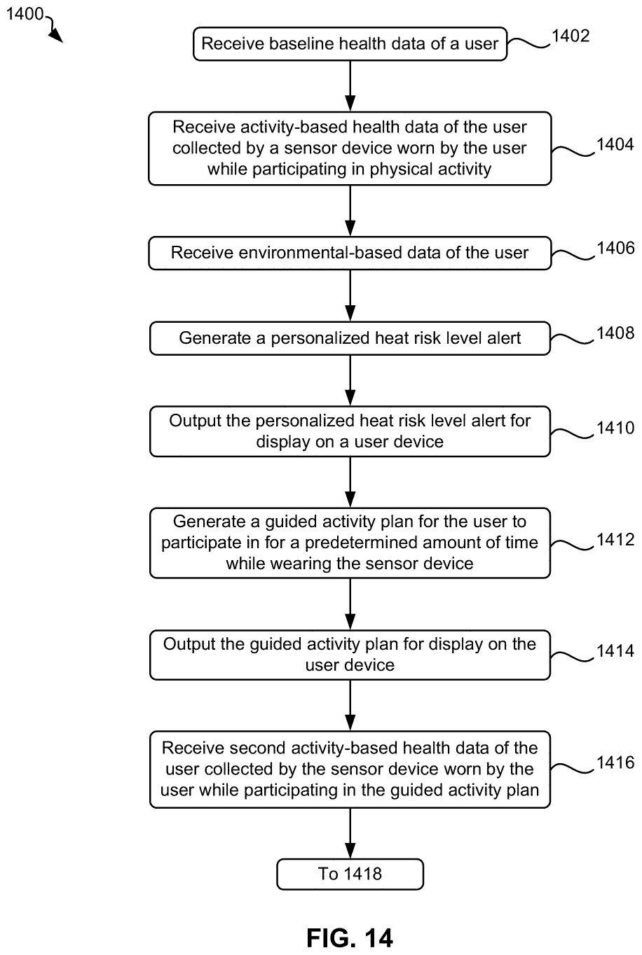

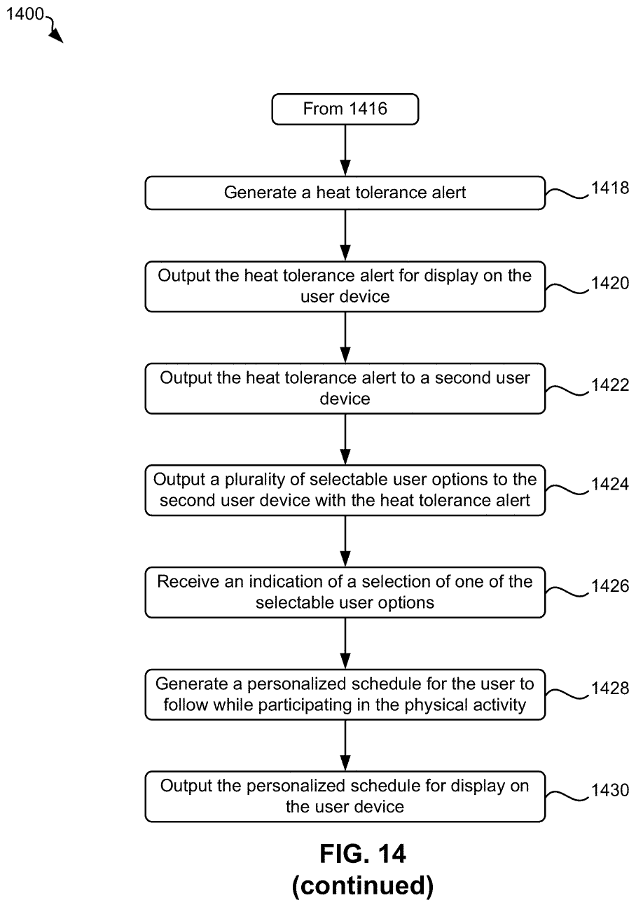

FIG. 14 shows a method 1400, in accordance with one embodiment. As an option, the present method 1400 may be implemented in devices such as those shown in the other FIGS. described herein. Of course, however, this method 1400 and others presented herein may be used to provide applications which may or may not be related to the illustrative embodiments listed herein. Further, the methods presented herein may be carried out in any desired environment. Moreover, more or less operations than those shown in FIG. 14 may be included in method 1400, according to various embodiments. It should also be noted that any of the aforementioned features may be used in any of the embodiments described in accordance with the various methods.

Operation 1402 includes receiving baseline health data of a user. Note that the received baseline health data may be pre-acquired, e.g., using a known type of health data input module, using a known technique for gathering health data from one or more sources, based on one or more questionnaires, processing images of a user using one or more known types of recognition techniques, etc. A non-limiting list of the baseline health data of the user may include, e.g., medical history data and record of previous heat injury or illness, anthropometric data such as weight and/or height, age, workday length and/or shift length, biological sex, a determined body mass index, etc.

Detection of Individual Heat Susceptibility at Baseline

Activity-based physiological and environmental data of the user, e.g., biometric data, collected by a sensor device worn by the user while participating in physical activity is received, e.g., see operation 1404 of method 1400. The sensor device may be similar to one or more of the sensor devices described elsewhere herein. For example, the sensor device may be worn on the upper arm and in one preferred approach is configured to be worn on the users arm by one or more hook and loop straps. Accordingly, in some approaches, the sensor device may be configured to continually, e.g., according to a predetermined interval, and non-invasively, collect predetermined metrics of the user using one or more known types of sensors, e.g., a camera, a heartbeat sensor, a temperature sensor for determining a temperature of the user, a humidity sensor, a motion sensor, a global positioning system, a proximity sensor, a gyroscope, an accelerometer, a microphone, a heat flux sensor, etc. Environmental data can also be obtained, e.g., received, via an external weather service (API).

The activity-based health data may depend on the approach. For example, in some approaches, the activity-based health data may include physiological and environmental data of the user, e.g., ambient temperature, ambient humidity, relative humidity, skin temperature of the user, skin humidity of the user measured by the sensor device, near skin humidity of the sensor, microclimate temperature and relative humidity, a perspiration rate of the user, a heart rate of the user such as measured via photoplethysmography or via one or more known techniques, an activity type that the user is determined to be engaged in and data associated therewith, a rate of movement of the user, core body temperature of the user, etc. In another approach, the activity-based health data may additionally and/or alternatively include motion data.

It should be noted that the type of physical activity that the activity-based health data is based on may depend on the approach. For example, in some approaches, the activity-based health data may be collected while the user is participating in non-stationary physical activity, e.g., running, walking, swimming, performing a work task, etc. In contrast, the activity-based health data of the user may additionally and/or alternatively be collected by the sensor device worn by the user while participating in a stationary activity, e.g., sleeping, taking a break at work, sitting stationary, awake in the user's home, driving to work, watching a user device, etc. As will be described in some approaches elsewhere herein, the data collected from the sensor data may be paired with user inputs, e.g., age, height, weight, medical history, clothing layers, and weather data, e.g., via API calls. In some approaches, these data together may serve as continuous inputs into the models for the heat acclimatization program, and the heat acclimatization detection and alert.

Operation 1406 includes receiving environmental-based data of the user collected by the sensor device worn by the user. Data based on clothing worn by the user and/or clothing expected to be worn by the user may additionally and/or alternatively be received in an optional operation of method 1400. The environmental-based data and/or the clothing-based data may be similar to and/or collected using similar techniques to the environmental-based data and/or the clothing-based data described elsewhere herein, e.g., see operation 305 of method 300, operation 706 of method 700 and 1106 of method 1100.

Operation 1408 includes generating a personalized heat risk level alert. In some preferred approaches, the personalized heat risk level alert includes a personalized heat risk level stratification category of the user. For example, in at least some of such approaches, the personalized heat risk level stratification category of the user may be low, e.g., it is determined that the user has no underlying conditions that would be relatively likely to cause the user to be at risk for heat-related injuries and/or illnesses. In another approach, the personalized heat risk level stratification category of the user may be moderate, e.g., it is determined that the user has at least one underlying condition that may cause the user to be at risk for heat related injuries and/or illnesses and therefore it may be beneficial to modify job tasks and/or a start/stop schedule of the user for participating in physical activity in order to prevent the user from experiencing a heat-related injury and/or illness. In yet another approach, the personalized heat risk level stratification category of the user may be high, e.g., it is determined that the user has at least two underlying conditions that may cause the user to be at risk for heat-related injuries and/or illnesses and therefore extreme care should be taken with the user to minimize heat exposure in order to prevent the user from experiencing a heat-related injury and/or illness.

Various techniques for generating the personalized heat risk level stratification of the user, and more specifically the personalized heat risk level stratification category of the user will now be described according to various approaches. In some approaches, the personalized heat risk level stratification category of the user may be determined using a table that includes a plurality of heat risk categories that are pre-associated to values of the received data. For example, in one approach, the personalized heat risk level stratification category of the user may be determined by accessing the table and determining which personalized heat risk level stratification category correspond to at least some values of the received data, e.g., preferably at least one of the baseline health data of the user, the activity-based health data of the user and the environmental-based data of the user.

The personalized heat risk level stratification category of the user may in some approaches additionally and/or alternatively be determined using data modeling. In one or more of such approaches a database may be generated by the data modeling using ongoingly collected and/or updated values of the received data. Data of the database may be comparatively analyzed with the received data of a user in order to determine a personalized heat risk level stratification category of the user. More specifically, in some approaches, the comparative analysis may include comparing data of the user to data of the database to identify personalized heat risk level stratification categories that were previously incorporated alerts of other users having data with at least a predetermined degree of similarity with the user. In such approaches, the more data that is incorporated into the modeling and database, the more likely that the determined personalized heat risk level of the user will be accurate.

In some approaches, the personalized heat risk level stratification category, e.g., heat risk level, of the user may additionally and/or alternatively be determined based on measured and/or predicted environmental values, e.g., such as the environmental-based data of the user collected by the sensor device worn by the user. One or more of such approaches may utilize API calls to a weather source and/or network connected environmental sensors. In one approach, such data may be incorporated into a data modeling technique described above in order to determine the personalized heat risk level stratification category of the user.

Data collection and research based on trial and analysis of results may additionally and/or alternatively be used for determining the personalized heat risk level stratification category of the user. Note that in one or more of such approaches, the trial process may be performed in a lab or any other controlled setting to ensure that users are not injured and/or do not become ill as a result of potential inaccuracies in the determination of a personalized heat risk level stratification category of a user. The trial and analysis of results may in one approach include changing the personalized heat risk level stratification category of the user from moderate to low or alternatively from high to moderate in response to a determination that the user does not experience injury and/or illness within a predetermined amount of time after being determined to have a personalized heat risk level stratification category of moderate or high (respectively). The trial and analysis of results may in another approach include changing the personalized heat risk level stratification category of the user from low to moderate or alternatively from moderate to high in response to a determination that the user experiences injury and/or illness within a predetermined amount of time after being determined to have a personalized heat risk level stratification category of low or moderate (respectively). In some approaches, received user feedback may be incorporated into the trial and analysis of results, e.g., such as a received selection by a user of at least one of a plurality of selectable user options asking, such as user feedback from the user as to whether a heat injury or illness occurred, etc.

Test case research and extrapolation based on results may additionally and/or alternatively be used for determining the personalized heat risk level stratification category of the user. For example, in some approaches, the received data of the user may be applied to one or more known techniques of test case research and extrapolation to determine the personalized heat risk level stratification category of the user. One or more known types of calculations may be additionally and/or alternatively be used for determining the personalized heat risk level stratification category of the user. For example, in some approaches, the received data of the user may be applied to a black box equation having an output that includes a determined personalized heat risk level stratification category of the user. In another approach, the received data of the user may be applied to a known type of machine learning algorithm to generate and/or ongoingly update the determined personalized heat risk level stratification category of the user.

Generating the personalized heat risk level alert may include adding the determined personalized heat risk level stratification category of the user to a known type of alert format. Operation 1410 of method 1400 includes outputting the personalized heat risk level alert for display on a user device. In some approaches the personalized heat risk level alert may be output close in time after the baseline health data of the user is received, e.g., subsequent to the user entering the baseline heath data in a medical history questionnaire. An explanation may be included in the personalized heat risk level alert that provides suggestions as to what degree the user should limit their participation in physical activity in accordance with the determined personalized heat risk level stratification category of the user. Such an explanation may include suggested tasks of a personalized schedule which will be described elsewhere herein. A predetermined user-friendly explanation for what the determined personalized heat risk level stratification category of the user means may also be included in the personalized heat risk level alert in some approaches. In some approaches, the personalized heat risk level alert may be output for display on a second user device, e.g., of a manager of the user. However, in some approaches, some information that is personal to the user may be excluded from an alert output to the second user device, e.g., such as the user's medical conditions. According to various approaches, the personalized heat risk level alert may be output to a user device via, e.g., an application, a dashboard, SMS, etc.

It should be noted that although method 1400 illustrates various types of received data continuing to operation 1410, in some approaches only some of such types of data may be used for generating the personalized heat risk level alert. Accordingly, in some approaches the personalized heat risk level alert may be generated and/or output based on one or more of the types of received data.

As briefly mentioned elsewhere herein, a user's heat tolerance may be at least partially based on the user's genetic makeup. Accordingly, in some approaches, in order to determine whether a user may be heat intolerant due to their genetic makeup and/or other intrinsic qualities, method 1400 may include generating a guided exercise activity for the user to participate in for a predetermined period of times, e.g., five minutes, fifteen minutes, two hours, etc. For example, a guided activity plan for the user to participate in for a predetermined amount of time while wearing the sensor device may be generated, e.g., see operation 1412. In some approaches, a predetermined guided exercise activity for the user to participate in may be used. In another approach, generating the guided exercise activity for the user to participate in may include accessing a table of guided exercise activities, where the table includes at least some predefined guided exercise activities and/or a predetermined amount of time that are pre-associated to values of the received data. More specifically, in such an approach, generating the guided exercise activity for the user to participate in may using at least some of the predefined guided exercise activities and/or predetermined amounts of times that are pre-associated to values of the received data. The predefined guided exercise activities may be based on a user's predefined schedule and/or a schedule determined using known techniques. This way, the guided exercise activity for the user to participate in may be incorporated into an activity that the user planned to participate in. For example, in response to a determination that the user is planning to buy groceries on a predetermined day, generating the guided exercise activity for the user to participate in may include the user using a predefined route to walk to and from the grocery store.

The guided activity plan is output for display on the user device, e.g., see operation 1414. The user's physiological data is preferably continuously monitored during the user's participation in the guided exercise activity. A non-limiting list of such data may include, e.g., skin temperature, motion, step rate, heart rate, skin humidity. The user's demographic data, e.g., age, weight, height, sex, etc., may additionally and/or alternatively be continuously monitored during the user's participation in the guided exercise activity.

Operation 1416 includes receiving activity-based health data of the user collected by the sensor device worn by the user while participating in the guided activity plan (hereafter referred to as “second activity-based health data of the user” in method 1400). The second activity-based health data of the user in some preferred approaches includes physiological data. For example, in some approaches, the second activity-based health data of the user includes, e.g., skin temperature, motion, step rate, heart rate, skin humidity, etc.

In some approaches, subsequent to a determination that the user has completed the guided exercise activity and/or subsequent to receiving the second activity-based health data of the user, the second activity-based health data of the user may be processed, e.g., on the user device, on the sensor device, on a second user device, etc., and based on results of the processing, the user may be assigned a binary heat tolerance classification. For example, operation 1418 includes generating a heat tolerance alert. IN some preferred approaches the heat tolerance alert may include a personalized heat tolerance stratification of the user that is based on the second activity-based health data of the user and the baseline health data of the user, e.g., demographic data that includes age, weight, height, sex, etc. The personalized heat tolerance stratification of the user may, in some approaches, be selected from personalized heat tolerance stratification categories, including heat tolerant and likely heat intolerant. According to various approaches, the personalized heat tolerance stratification of the user may be determined using techniques including, e.g., via machine learning methods such as decision trees, logistic regression, a lookup table, or black box models, etc., using the received activity-based health data of the user. Of course, the personalized heat tolerance stratification category of the user may change at any time, e.g., as a result of the user electing to repeat the guided activity plan, as a result of the user performing another guided activity plan, as a result of the user having a change in weight or any other health condition, etc.

Operation 1420 includes outputting the heat tolerance alert for display on the user device.

As recited in operation 1422, the heat tolerance alert may additionally and/or alternatively be output to a second user device, e.g., for display on the second user device. The second user device may be of a second user that manages the user. In some approaches outputting the heat tolerance alert to the second user device may enable the second user to interject input, e.g., type, relative rigorousness, time, etc., regarding physical activity that the user may thereafter be assigned. The outputting of the heat tolerance alert to the second user device may enable a user of the second device, such as a manager, to manage a workforce, e.g., enabling the placement of more heat tolerant workers in relatively hotter or tougher work environments. Moreover, indoor jobs may be reserved for generally heat intolerant users (under air conditioned control for example) and/or users determined to have relatively less protective clothing layers to maximize heat loss. Furthermore, outputting of the heat tolerance alert to the second user device may enable the grouping of heat intolerant workers together as they may have a more conservative work/rest schedule that requires more frequent rest periods, whereas the heat-tolerant workers can be grouped together based on their ability to work potentially longer periods of time without rest while maintaining lower core temperatures. Such groupings may be generated based on determined similarities between different workers, and the groupings may be included as suggested groupings and/or worker classifications may be output to the user device and/or the second user device. For example, operation 1424 includes outputting a plurality of selectable user options to the second user device with the heat tolerance alert. In one approach, a first of the selectable user options may correspond to a first assignment of participating in the physical activity, while a second of the selectable user options corresponds to a second assignment of participating in the physical activity. In such an approach the first assignment may be relatively more physically rigorous than the second assignment. According to another approach, the plurality of selectable user options may be output to a dashboard and/or an application of a user device.

Operation 1426 includes receiving an indication of a selection of one of the selectable user options, e.g., receiving from the second user device. In response to receiving, from the second user device, an indication of a selection of one of the selectable user options, a personalized schedule for the user to follow while participating in the physical activity may be generated, e.g., see operation 1428. A relative rigorousness of the generated personalized schedule may be based on the indicated selection of the one of the selectable user options, e.g., based on whether the first or the second selectable user options were selected. In other words, in response to a determination that the indication includes information that indicates that the first selectable user option was selected, the personalized schedule may be generated to include a relatively more rigorous personalized schedule. In contrast, in response to a determination that the indication includes information that indicates that the second selectable user option was selected, the personalized schedule may be generated to include a relatively less rigorous personalized schedule. The personalized schedule may in some approaches additionally and/or alternatively be generated using techniques similar to those described elsewhere herein for generating a personalized schedule.

Operation 1430 includes outputting the personalized schedule for display on the user device.

It should be noted that as a result of the personalized schedule being based on health data and the personalized heat risk level alert of the user, user injuries and/or illnesses that would otherwise occur using conventional heat coping techniques are avoided. This is because a user individualized heat susceptibility alert system has heretofore not been considered and/or incorporated into conventional heat coping efforts. As mentioned elsewhere above, this is because conventional techniques for coping with relatively hot environments only consider a limited number of variables among workers, and ultimately do not account for the large physiological variability among individuals, e.g., genetic differences, biological sex, age, fitness level, acclimatization status, whether the worker is taking medication, what type and/or dosage of medication a worker is taking, etc. Moreover, conventional techniques for coping with relatively hot environments are often designed from laboratory tests that utilize subject populations predominantly comprised of young, healthy men. Accordingly, typical heat coping techniques are not accurate for women and older individuals, e.g., such as women and individuals more than thirty-five years old. This does not ensure all workers are scheduled to participate in physical activity in a way that prevents heat-related injuries and/or illnesses. Accordingly, users continue to be susceptible to heat-related injuries. In sharp contrast to the deficiencies described above, various embodiments and approaches described herein ensure that the user's lifestyle factors and current medical status (e.g., medications that the user is taking and/or prescribed and/or underlying diseases that the user has) are incorporated into a personalized schedule for the user to follow while participating in the physical activity. Overall, the techniques of various approaches described herein are optionally specific to each individual user's uniqueness. Accordingly, the inventive discoveries disclosed herein with regards to individualized heat susceptibility classification proceed contrary to conventional wisdom.

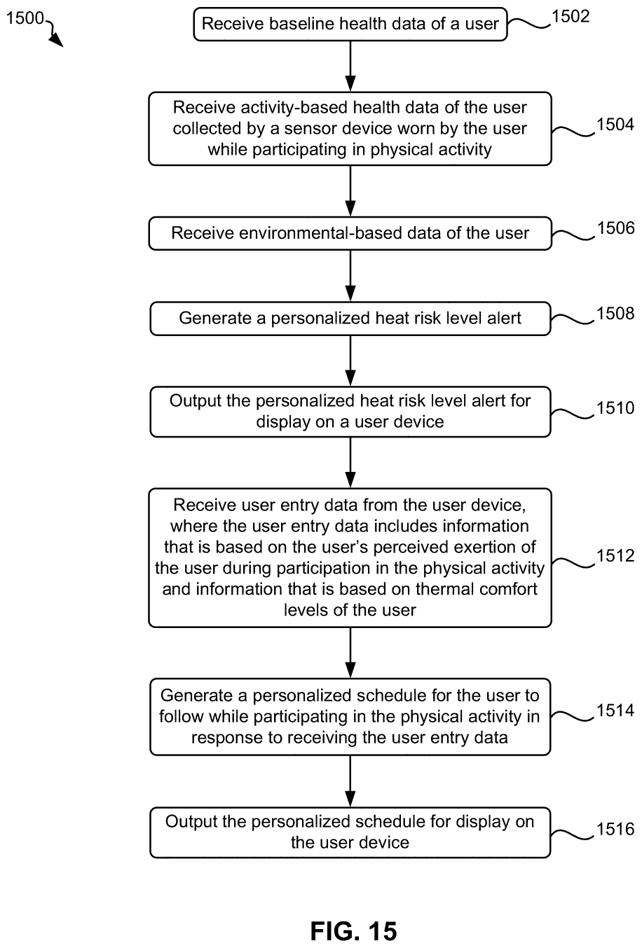

FIG. 15 shows a method 1500, in accordance with one embodiment. As an option, the present method 1500 may be implemented in devices such as those shown in the other FIGS. described herein. Of course, however, this method 1500 and others presented herein may be used to provide applications which may or may not be related to the illustrative embodiments listed herein. Further, the methods presented herein may be carried out in any desired environment. Moreover, more or less operations than those shown in FIG. 15 may be included in method 1500, according to various embodiments. It should also be noted that any of the aforementioned features may be used in any of the embodiments described in accordance with the various methods.

Detection of Individual Heat Susceptibility Each Morning

Operation 1502 includes receiving baseline health data of a user. Activity-based health data of the user collected by a sensor device worn by the user while participating in physical activity is also received, e.g., see operation 1504. Moreover, environmental-based data of the user may be received, e.g., see operation 1506.

A personalized heat risk level alert may be generated, e.g., see operation 1508. The personalized heat risk level alert may in some approaches include a personalized heat risk level stratification of the user at a future predetermined period of time. The personalized heat risk level stratification of the user may be based on the baseline health data of the user, the activity-based health data of the user and the environmental-based data of the user. In some preferred approaches the stratification of the user may be selected from the personalized heat risk level stratification categories including, e.g., low, moderate and high. The personalized heat risk level alert is output for display on a user device, e.g., see operation 1510.

Operation 1512 includes receiving user entry data from the user device. The user entry data includes information that is based on the user's perceived exertion of the user during participation in the physical activity and information that is based on thermal comfort levels of the user. Such data may be entered by the user selecting one or more selectable user options that are output with the personalized heat risk level alert, e.g., a relative degree of alertness, a relative degree of fatigue, a relative degree of alertness, a relative feeling of happiness, etc. For context, it should be noted that heat load may be cumulative throughout a week and therefore increases risk for heat-related injuries and illnesses. This risk may be dependent on the user's age, but also the user's workload and environmental (work) conditions. It is important that throughout the work week, users and their managers are aware of increasing heat risk that may result from the user's previous days of work so that they can modify the workload or remove the user from the heat altogether (if needed) so that heat injuries and illnesses can be avoided. Accordingly, in response to receiving the user entry data, a personalized schedule for the user to follow while participating in the physical activity may be generated, e.g., see operation 1514. A relative rigorousness of the generated personalized schedule is preferably based on the information of the user entry data. Operation 1516 includes outputting the personalized schedule for display on the user device.

Method 1500 may include providing daily (each morning) updates to a second user, e.g., a manager, and/or the user about a heat risk level of the user, e.g., low, moderate, high, etc., via any of the mediums described elsewhere herein. This notification of the risk level may be generated using similar techniques to those described in operation 1408 of method 1400, and may be output in addition to a baseline heat risk category of the user. This notification provides additional information about the user's risk that day specifically. Accordingly, the notification may be based on the user's physiological data collected from the sensor device (or other such device that collects similar data), along with the weather data each day (temperature and humidity pulled in from an API call). Machine learning models (such as decision trees), lookup tables, and/or black box models will be used to analyze the user's physiological data from the current day of work to predict the user's heat risk the following workday. In some preferred approaches, these models incorporate not only the previous day of work, but all of the preceding workdays of a given week, e.g. assuming it is currently Wednesday, the model will integrate the data from the user on Monday and Tuesday to give a prediction of the worker's heat risk on Wednesday. A perceived exertion and thermal comfort levels of the user may be determined, e.g., via surveys in an application, dashboard, SMS, etc., and additionally and/or alternatively incorporated into these models to capture the user's perception of their work output and heat load each day. In this way, the user's heat risk is be up to date each morning and is an integrative view of their work week. This heat risk stratification is be provided to managers and the user, so that combined with the user's baseline stratification, managers are enabled to make informed decisions about users that they manage and teams that they manage to minimize heat-related injuries and illnesses at a job site.

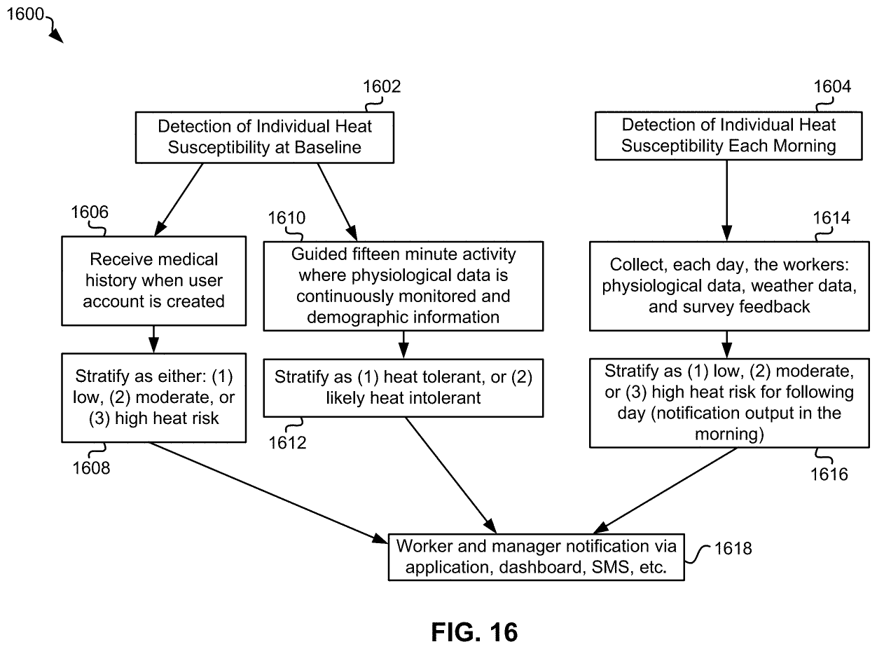

FIG. 16 shows a method 1600, in accordance with one embodiment. As an option, the present method 1600 may be implemented in devices such as those shown in the other FIGS. described herein. Of course, however, this method 1600 and others presented herein may be used to provide applications which may or may not be related to the illustrative embodiments listed herein. Further, the methods presented herein may be carried out in any desired environment. Moreover, more or less operations than those shown in FIG. 16 may be included in method 1600, according to various embodiments. It should also be noted that any of the aforementioned features may be used in any of the embodiments described in accordance with the various methods.

It should be noted that the flowchart of method 1600 illustrates an overview of the relationships between techniques for detection of individual heat susceptibility at baseline, e.g., see 1602, and techniques for detection of individual heat susceptibility each morning, e.g., see 1604.

Operation 1606 includes receiving medical history and/or current lifestyle factors, e.g., an amount that a user smokes, an amount that a user drinks, a frequency in which the user overeats, a frequency in which the user is sleep deprived, whether the user is diabetic, whether the user has heart disease, etc., when a worker account is created. Note that current lifestyle factors may be applied similar to how the medical history information is described to be applied elsewhere herein. In some approaches, such information may be received subsequent to outputting questions to a user device requesting such data. In operation 1608, the is stratified as either: (1) low, (2) moderate, or (3) high heat risk. In operation 1618 the stratification is output to the worker and/or a manager of the worker in a notification, e.g., via an application, a dashboard, SMS, etc.

In operation 1610 a guided fifteen-minute activity is generated for the worker where physiological data is continuously monitored and demographic information. Moreover, the worker is stratified as: (1) heat tolerant, or (2) likely heat intolerant, e.g., see operation 1612. The stratification is output to the worker and/or a manager of the worker in a notification, e.g., via an application, a dashboard, SMS, etc., in operation 1618. Note that the notification may be different than the notification mentioned above with regards to outputting the stratification of operation 1608.

Operation 1614 includes collecting, each day, the workers: physiological data, weather data, and survey feedback. Such data may be used to stratify the worker as (1) low, (2) moderate, or (3) high heat risk for following day (notification output in the morning), e.g., see operation 1616. The stratification is output to the worker and/or the manager of the worker in a notification, e.g., via an application, a dashboard, SMS, etc., in operation 1618. Note that the notification may be different than the notification mentioned above with regards to outputting the stratification of operation 1608 and the notification mentioned above with regards to outputting the stratification of operation 1612.

The inventive concepts disclosed herein have been presented by way of example to illustrate the myriad features thereof in a plurality of illustrative scenarios, embodiments, and/or implementations. It should be appreciated that the concepts generally disclosed are to be considered as modular, and may be implemented in any combination, permutation, or synthesis thereof. In addition, any modification, alteration, or equivalent of the presently disclosed features, functions, and concepts that would be appreciated by a person having ordinary skill in the art upon reading the instant descriptions should also be considered within the scope of this disclosure.

While various embodiments have been described above, it should be understood that they have been presented by way of example only, and not limitation. Thus, the breadth and scope of an embodiment of the present invention should not be limited by any of the above-described exemplary embodiments, but should be defined only in accordance with the following claims and their equivalents.

What is claimed is:

1. A method, comprising:
receiving baseline health data of a user;
receiving activity-based health data of a user collected by a sensor device worn by the user while participating in physical activity;
receiving environmental-based data of the user collected by the sensor device worn by the user and/or from an external application programming interface (API);
generating a personalized schedule for the user to follow while participating in the physical activity based on the baseline health data of the user, the activity-based health data of the user and the environmental-based data of the user,
wherein the personalized schedule includes at least one instruction of an amount of fluids for the user to consume while participating in the physical activity to maintain hydration of the user and prevent dehydration of the user; and
outputting the personalized schedule for display on the user device.

2. A method as recited in claim 1, wherein the baseline health data is selected from the group of data consisting of: medical history data, anthropometric data, age, workday length and biological sex.

3. A method as recited in claim 1, wherein the activity-based health data is selected from the group of data consisting of: heart rate data, temperature data, motion data and humidity data.

4. A method as recited in claim 1, wherein the activity-based health data is collected by the sensor device worn by the user while participating in stationary activity.

5. A method as recited in claim 1, wherein the personalized schedule includes at least one instruction of when to start participating in the physical activity and at least one instruction of when to stop participating in the physical activity in order to maintain hydration of the user and prevent dehydration of the user.

6. A method as recited in claim 1, comprising: outputting an alert to the user device of the user to remind the user to start participating in the physical activity; and outputting an alert to the user device of the user to remind the user to stop participating in the physical activity.

7. A method as recited in claim 1, comprising: outputting a user entry option with the personalized schedule, wherein the entry user option requests an amount of fluid that the user consumed within a predetermined period of time in accordance with the at least one instruction of the amount of fluids for the user to consume; receiving a response to the user entry option; updating the personalized schedule based on information of the received response; and outputting the updated personalized schedule for display on the user device.

8. A method as recited in claim 7, wherein updating the personalized schedule based on information of the received response includes: determining whether the information of the received response indicates that the amount of fluid that the user consumed within the predetermined period of time is at least the amount of fluids of the instruction; and in response to a determination that the information of the received response indicates that the amount of fluid that the user consumed within the predetermined period of time is not at least the amount of fluids of the instruction, increasing an amount of fluids of an instruction of the updated personalized schedule.

9. A computer program product, comprising: a computer readable storage medium having stored thereon computer readable program instructions configured to cause a processor of a computer system to:
receive, by the processor, baseline health data of a user;
receive, by the processor, activity-based health data of a user collected by a sensor device worn by the user while participating in physical activity;
receive, by the processor, environmental-based data of the user collected by the sensor device worn by the user and/or from an external application programming interface (API);
generate, by the processor, a personalized schedule for the user to follow while participating in the physical activity based on the baseline health data of the user, the activity-based health data of the user and the environmental-based data of the user,
wherein the personalized schedule includes at least one instruction of an amount of fluids for the user to consume while participating in the physical activity to maintain hydration of the user and prevent dehydration of the user; and
output, by the processor, the personalized schedule for display on the user device.

10. A computer program product as recited in claim 9, wherein the baseline health data is selected from the group of data consisting of: medical history data, anthropometric data, age, workday length and biological sex.

11. A computer program product as recited in claim 9, wherein the activity-based health data is selected from the group of data consisting of: heart rate data, temperature data, motion data and humidity data.

12. A computer program product as recited in claim 9, wherein the activity-based health data is collected by the sensor device worn by the user while participating in stationary activity.

13. A computer program product as recited in claim 9, wherein the personalized schedule includes at least one instruction of when to start participating in the physical activity and at least one instruction of when to stop participating in the physical activity in order to maintain hydration of the user and prevent dehydration of the user.

14. A computer program product as recited in claim 9, the computer readable program instructions configured to cause the processor of the computer system to:
output, by the processor, an alert to the user device of the user to remind the user to start participating in the physical activity; and output, by the processor, an alert to the user device of the user to remind the user to stop participating in the physical activity.

15. A computer program product as recited in claim 9, the computer readable program instructions configured to cause the processor of the computer system to:
output, by the processor, a user entry option with the personalized schedule, wherein the entry user option requests an amount of fluid that the user consumed within a predetermined period of time in accordance with the at least one instruction of the amount of fluids for the user to consume; receive, by the processor, a response to the user entry option; update, by the processor, the personalized schedule based on information of the received response; and output, by the processor, the updated personalized schedule for display on the user device.

16. A computer program product as recited in claim 15, wherein updating the personalized schedule based on information of the received response includes:
determining whether the information of the received response indicates that the amount of fluid that the user consumed within the predetermined period of time is at least the amount of fluids of the instruction; and in response to a determination that the information of the received response indicates that the amount of fluid that the user consumed within the predetermined period of time is not at least the amount of fluids of the instruction, increasing an amount of fluids of an instruction of the updated personalized schedule.

17. A system, comprising:
a processor; and
logic integrated with and/or executable by the processor, the logic being configured to:
receive baseline health data of a user;
receive activity-based health data of a user collected by a sensor device worn by the user while participating in physical activity;
receive environmental-based data of the user collected by the sensor device worn by the user and/or from an external application programming interface (API);
generate a personalized schedule for the user to follow while participating in the physical activity based on the baseline health data of the user, the activity-based health data of the user and the environmental-based data of the user,
wherein the personalized schedule includes at least one instruction of an amount of fluids for the user to consume while participating in the physical activity to maintain hydration of the user and prevent dehydration of the user; and
output the personalized schedule for display on the user device.

18. A system as recited in claim 17, wherein the baseline health data is selected from the group of data consisting of: medical history data, anthropometric data, age, workday length and biological sex.

19. A system as recited in claim 17, wherein the activity-based health data is selected from the group of data consisting of: heart rate data, temperature data, motion data and humidity data.

20. A system as recited in claim 17, wherein the activity-based health data is collected by the sensor device worn by the user while participating in stationary activity.
